(19) United States
(12) Patent Application Publication
(10) Pub. No.: US 2026/0034124 A1
(43) Pub. Date: Feb. 5, 2026

(54) NOVEL SALTS AND CRYSTALS

(71) Applicant: INTRA-CELLULAR THERAPIES, INC., Bedminster, NJ (US)

(72) Inventor: Peng LI, New Milford, NJ (US)

(21) Appl. No.: 19/099,759

(22) PCT Filed: Jul. 28, 2023
(86) PCT No.: PCT/US2023/071269

§ 371 Date: Jan. 29, 2025

Related U.S. Application Data
(60) Provisional application No. 63/393,911, filed on Jul. 30, 2022.

(51) Int. Cl. A61K 31/4985; A61K 47/10; A61K 47/38; C07D 487/16

(52) U.S. Cl. CPC A61K 31/4985; A61K 47/10; A61K 47/38; C07D 487/16

(57) ABSTRACT

The disclosure provides new, stable, crystalline salt forms of lumateperone, together with methods of making and using them, and pharmaceutical compositions comprising them.

### CROSS-REFERENCE TO RELATED APPLICATIONS

This application is a PCT International Application which claims priority to, and the benefit of, U.S. Provisional Application Ser. No. 63/393,911, filed on Jul. 30, 2022, the contents of which are hereby incorporated by reference in its entirety.

### FIELD

This disclosure relates to certain novel salts and crystal forms of lumateperone, a substituted heterocycle fused gamma-carboline, the manufacture thereof, pharmaceutical compositions thereof, and use thereof, e.g., in the treatment of diseases or abnormal conditions involving or mediated by the 5-HT_{2A} receptor, serotonin transporter (SERT), and/or dopamine D₁/D₂ receptor signaling pathways.

### BACKGROUND

The substituted heterocycle fused gamma-carboline compound lumateperone, with the chemical name (4-((6bR,10aS)-3-methyl-2,3,6b,9,10,10a-hexahydro-1H-pyrido[3′,4′: 4,5]pyrrolo[1,2,3-de]quinoxalin-8(7H)-yl)-1-(4-fluorophenyl)-1-butanone), is known to be a serotonin receptor (5-HT_{2A}), dopamine receptor (D1 and/or D2), and serotonin transporter (SERT) ligand, and it is useful in treating a variety of central nervous system disorders. It is also known as ITI-007, and it has the following structure:

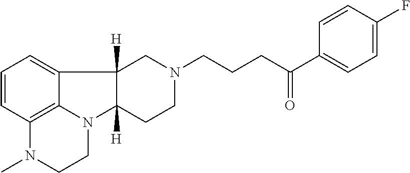 [SMILES: CN1CCN2c3c(cccc31)[C@@H]1CN(CCCC(=O)c3ccc(F)cc3)CC[C@@H]12]

Lumateperone antagonizes the serotonin-2A (5-HT_{2A}) receptor, and/or modulates dopamine receptor signaling at the level of key intra-cellular phosphoproteins. This compound is principally known to be useful for the treatment of positive and negative symptoms of schizophrenia, depression (especially acute depression and bipolar depression), anxiety and traumatic disorders (including acute anxiety and post-traumatic stress disorder), and dementias (including Alzheimer's disease and the symptoms associated therewith). At dopamine D2 receptors, this compound has dual properties and acts as both a post-synaptic antagonist and a pre-synaptic partial agonist of the D2 receptor. It also stimulates phosphorylation of glutamatergic NMDA NR2B, or GluN2B, receptors in a mesolimbic specific manner. It is believed that this regional selectivity in the brain areas thought to mediate the efficacy of antipsychotic drugs, together with the serotonergic, glutamatergic, and dopaminergic interactions, may result in antipsychotic efficacy for positive, negative, affective, and cognitive symptoms associated with schizophrenia. The compound also exhibits serotonin reuptake inhibition, providing antidepressant activity for the treatment of schizoaffective disorder, co-morbid depression, and/or as a stand-alone treatment for major depressive disorder, bipolar depression, and treatment-resistant depression. Lumateperone is also useful for the treatment of bipolar disorder and other psychiatric and neurodegenerative disorders, particularly behavioral disturbances associated with dementia, autism, and other CNS diseases. These features may be able to improve the quality of life of patients with schizophrenia and enhance social function to allow them to more fully integrate into their families and their workplace.

Lumateperone displays differential dose-dependent effects, selectively targeting the 5-HT_{2A} receptor at low doses, while progressively interacting with the D2 receptor at higher doses. As a result, at lower doses, it is useful in treating sleep, aggression, and agitation. At a high dose, it can treat acute exacerbated and residual schizophrenia, bipolar disorders, and mood disorders.

Lumateperone is a potent (Ki=0.5 nM) 5-HT_{2A} receptor antagonist, and has activity as a mesolimbic/mesocortical-selective dopamine receptor protein phosphorylation modulator consistent with presynaptic D2 receptor partial agonism and postsynaptic D2 receptor antagonism (Ki=32 nM) in vivo, high D1 receptor affinity (Ki=52 nM), and inhibition of the serotonin transporter (SERT) (Ki=26-62 nM, using different assays for SERT activity), but negligible binding to receptors (e.g., H1 histaminergic, 5-HT2C, and muscarinic) associated with cognitive and metabolic side effects of antipsychotic drugs.

Lumateperone tosylate (Caplyta®) is currently approved in the United States for the treatment of schizophrenia and bipolar depression. It is currently in clinical trials and development for additional indications, including major depressive disorder (MDD).

It has also recently been found that lumateperone may be particularly effective in treating acute depression and acute anxiety owing to its rapid onset of action compared to existing antidepressants. This is believed to be due to its signaling through a neurotransmitter system separate from the traditional monoamine signaling systems. Lumateperone provides a dopamine D1 receptor-dependent enhancement of NMDA and AMPA currents coupled with activation of the mTOR (e.g., mTORC1) signaling pathway.

The formulation of lumateperone as a drug is difficult. In free base form, ITI-007 is an oily, sticky solid, with poor aqueous solubility. Making salts of the compound has proven to be unusually difficult. A hydrochloride salt form of lumateperone, prepared briefly during synthesis of the free base, was disclosed in WO 2000/0770020, but this particular salt was not crystalline, and such hydrochloride salts were found to be hygroscopic and showed poor stability. A stable, crystalline toluenesulfonic acid addition salt (monotosylate) of lumateperone was finally identified and described in, e.g., WO 2009/114181. Additional salts and polymorphs of lumateperone have since been described, including bistosylate, besylate, naphthalenesulfonate, and naphthalenedisulfonate salts. See, e.g., WO 2018/031535, WO 2019/102240, WO 2020/182988, WO 2020/112941, and IN2017/41021763.

While it is normally the case that a pharmaceutically acceptable salt form of a drug should have a high aqueous solubility, this is not always the most desirable form. For example, while drugs for oral, transmucosal, and intravenous delivery should have high water solubility, there is also a need for salt forms with low aqueous solubility, particularly for delivery from a long-acting injectable depot (e.g., for sustained delivery over a period of weeks or months). The low aqueous solubility of the drug in this situation results in slow dissolution of the drug from its insoluble, pharmacologically inactive depot form, to its pharmacologically active soluble form. Such a delivery mechanism requires high chemical and physical stability, however, so that the drug does not degrade in vivo to chemical products or polymorphs having unpredictable pharmacological or pharmacokinetic properties.

There is thus a need for chemically and physically stable, preferably crystalline, solid pharmaceutically acceptable salt form of lumateperone having low aqueous solubility.

### BRIEF SUMMARY

In an effort to find new chemically and physically stable, preferably crystalline, solid pharmaceutically acceptable salt forms of lumateperone having low aqueous solubility, an extensive salt screen was undertaken. Based on the known tosylate and besylate salts, it was believed that other aromatic sulfonate salts of lumateperone might be suitable. However, lumateperone does not readily form salts with other common, pharmaceutically acceptable acids. See, e.g., US 2019/0112309, US 2020/247805, and US 2020/015700. These references disclose attempts to make new crystalline salt forms of lumateperone with very low rates of success. For example, US 2019/112309 and US 2020/247805 disclose the results of a series of salt screening experiments performed using 90 counterions, six solvents, and four crystallization methods. A total of 624 combinations of counterion, solvent, and method were tested, and this resulted in the reproducible formation of only four stable, crystalline salts (an oxalate salt, a cyclamate salt, a 4-aminosalicylate salt, and three polymorphs of a hydrochloride salt). Importantly, it was not predictable which kind of counterion could form a stable crystalline salt, nor which method would successfully result in each stable crystalline salt. The vast majority of reaction conditions were found to result in either no salt formation, or the formation of an amorphous solid or oily liquid salt.

Now, following extensive screening and experimentation involving 36 different sulfonate counterions (aryl, heteroaryl, heterocycloalkyl, and alkyl sulfonic acids) using both 1:1 and 1:2 molar ratios, and several solvents (9:1 acetonitrile/water, methanol, ethyl acetate, and toluene), sixteen new candidate crystalline lumateperone salt were identified. On further scale up and confirmation, eight new, reproducible, stable, crystalline salt forms of lumateperone have been discovered. In particular, two new low-solubility salt forms of lumateperone were discovered having an aqueous solubility under 2 mg/mL: 4-t-butylbenzenesulfonate salt and 4-octylbenzenesulfonate salt. These salt forms are chemically and physically stable, crystalline, and have low aqueous solubility, and are this ideally suited to new pharmaceutical compositions for sustained or delayed release, such as long-acting injectable compositions.

Therefore, the present disclosure provides new salts forms of lumateperone, particularly new low-aqueous-solubility salt forms of lumateperone, which are especially advantageous for use in the preparation of long-acting injectable formulations, together with methods of making and using the same. Because of their low aqueous solubility, these new salt forms would not be expected to be suitable for traditional oral, immediate release preparations of lumateperone, such as tablets or capsules, nor would they be suitable for immediate release transmucosal formulations (e.g., sublingual tablets), immediate release subcutaneous injectable formulations, intravenous formulations, or sustained or delayed release oral formulations. However, they would be very useful for a variety of sustained or delayed release injectable pharmaceutical formulations.

Further areas of applicability of the present disclosure will become apparent from the detailed description provided hereinafter. It should be understood that the detailed description and specific examples, while indicating preferred embodiments of the disclosure, are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 depicts the X-ray powder diffraction (XPRD) pattern for the 4-octylbenzenesulfonate salt of lumateperone prepared according to Example 2.

FIG. 2 depicts the X-ray powder diffraction (XPRD) pattern for the 4-tert-butyllbenzenesulfonate salt of lumateperone prepared according to Example 3.

FIG. 3 depicts the X-ray powder diffraction (XPRD) pattern for the 4-propylbenzenesulfonate salt of lumateperone prepared according to Example 4.

FIG. 4A depicts the X-ray powder diffraction (XPRD) pattern for the 4-ethylbenzenesulfonate salt of lumateperone prepared according to Example 5A (Polymorph 1).

FIG. 4B depicts the X-ray powder diffraction (XPRD) pattern for the 4-ethylbenzenesulfonate salt of lumateperone prepared according to Example 5B (Polymorph 2).

FIG. 4C depicts the X-ray powder diffraction (XPRD) pattern for the 4-ethylbenzenesulfonate salt of lumateperone prepared according to Example 5C (Polymorph 3).

FIG. 5 depicts the X-ray powder diffraction (XPRD) pattern for the 2-naphthalenesulfonate salt of lumateperone prepared according to Example 6.

FIG. 6A depicts the X-ray powder diffraction (XPRD) pattern for the besylate salt of lumateperone prepared according to Example 7A (Polymorph 1).

FIG. 6B depicts the X-ray powder diffraction (XPRD) pattern for the besylate salt of lumateperone prepared according to Example 7B (Polymorph 2).

FIG. 6C depicts the X-ray powder diffraction (XPRD) pattern for the besylate salt of lumateperone prepared according to Example 7C (Polymorph 3).

FIG. 7 depicts the X-ray powder diffraction (XPRD) pattern for the pentane-1-sulfonate salt of lumateperone prepared according to Example 8.

FIG. 8 depicts the X-ray powder diffraction (XPRD) pattern for the heptane-1-sulfonate salt of lumateperone prepared according to Example 9.

FIG. 9 depicts the proton-NMR spectrum for the 4-octylbenzenesulfonate salt of lumateperone prepared according to Example 2.

FIG. 10 depicts the proton-NMR spectrum for the 4-tert-butylbenzenesulfonate salt of lumateperone prepared according to Example 3.

FIG. 11 depicts the proton-NMR spectrum for the 4-propybenzenesulfonate salt of lumateperone prepared according to Example 4.

FIG. 12A depicts the proton-NMR spectrum for the 4-ethylbenzenesulfonate salt of lumateperone Polymorph 1 prepared according to Example 5A.

FIG. 12B depicts the proton-NMR spectrum for the 4-ethylbenzenesulfonate salt of lumateperone Polymorph 2 prepared according to Example 5B.

FIG. 12C depicts the proton-NMR spectrum for the 4-ethylbenzenesulfonate salt of lumateperone Polymorph 3 prepared according to Example 5C.

### DETAILED DESCRIPTION

The following description of the preferred embodiment(s) is merely exemplary in nature and is in no way intended to limit the inventions described in the present disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The present disclosure generally provides new salt forms of lumateperone, and in particular new crystalline salt forms of lumateperone. Preferably, these new crystalline salt forms have a low aqueous solubility (e.g., at pH 7 or at pH 7.4). For example, salts according to the present disclosure may have aqueous solubilities of less than 20 mg/mL, e.g., less than 15 mg/mL, or less than 10 mg/mL, or less than 5 mg/mL, or less than 3 mg/mL, or less than 2 mg/mL, or less than 1 mg/mL, or less than 0.5 mg/mL, or less than 0.1 mg/mL, and/or at least 0.001 mg/mL, or at least 0.01 mg/mL, or at least 0.1 mg/mL, or at least 1 mg/mL.

In a first embodiment, the present disclosure provides lumateperone in the form of a 4-octylbenzenesulfonic acid addition salt (Salt 1). In further embodiments of Salt 1, the present disclosure provides:
1.1. Salt 1 in solid form.
1.2. Salt 1 or 1.1 in crystalline form, e.g., dry crystalline form.
1.3. Salt 1, 1.1, or 1.2, wherein the salt has about a 1:1 molar ratio of lumateperone to 4-octylbenzenesulfonic acid (i.e., a mono-4-octylbenzenesulfonate salt).
1.4. Salt 1, 1.1, or 1.2, wherein the salt has about a 1:2 molar ratio of lumateperone to 4-octylbenzenesulfonic acid (i.e., a bis-4-octylbenzenesulfonate salt).
1.5. Any foregoing form of Salt 1 which is a solvate, e.g., an ethyl acetate or a toluene solvate.
1.6. Any foregoing form of Salt 1 which is not a solvate.
1.7. Any foregoing form of Salt 1 which is a hydrate.
1.8. Any foregoing form of Salt 1 which is not a hydrate.
1.9. Any foregoing form of Salt 1 formed by combining lumateperone free base and 4-octylbenzenesulfonic acid in a molar ratio from 1:0.5 to 1:3, e.g., a 1:0.75 to 1:1.5 molar ratio, or a 1:0.75 to 1.25 molar ratio, or a 1:1.5 to 1:2.5 molar ratio, or a 1:1.75 to 1:2.25 molar ratio, or a 1:1.75 to 1:3 molar ratio, or a 1:1 to 1:2 molar ratio, or about a 1:1 molar ratio, or about a 1:1.5, or about a 1:2 molar ratio, or about a 1:2.5 molar ratio.
1.10. Any foregoing form of Salt 1 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. %, of amorphous forms.
1.11. Any foregoing form of Salt 1 in crystalline form, when crystallized from a mixture of 4-octylbenzenesulfonic acid and lumateperone free base, e.g., in an organic solvent, e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof; e.g., ethyl acetate or toluene, optionally wherein the lumateperone free base and the 4-octylbenzenesulfonic acid are in a molar ratio of about 1:1 or about 1:2.
1.12. Salt 1.11, wherein the salt is crystallized from the solvent after an anti-solvent is added, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane.
1.13. Any foregoing form of Salt 1, wherein the salt is formed from a 1:1 molar ratio of lumateperone free base to 4-octylbenzenesulfonic acid in ethyl acetate solvent, or from a 1:2 molar ratio of lumateperone free base to 4-octylbenzenesulfonic acid in ethyl acetate solvent, or from a 1:2 molar ratio of lumateperone free base to 4-octylbenzenesulfonic acid in toluene solvent.
1.14. Salt 1.13, wherein a proton-NMR analysis of the salt shows a molar ratio of lumateperone to 4-octylbenzenesulfonic acid of about 1:1.
1.15. Salt 1.13 or 1.14, wherein a DSC analysis shows one endothermic event at about 156° C. (e.g., a melt), or one endothermic event at about 164° C. (e.g., a melt), or one endothermic event at about 135° C. (e.g., a melt).
1.16. Any form of Salt 1.13-1.15, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 1, 4, 5, 9, 11, 12, 15, 17, and/or 18:

XRPD (Cu anode, Ni filter) for 4-
Octylbenzenesulfonate Salt Crystal

| # | Angle | d Value | Rel. Intensity |
|---|---|---|---|
| 1 | 9.086 | 9.72473 | 18.60% |
| 2 | 9.192 | 9.6129 | 6.40% |
| 3 | 11.369 | 7.77699 | 13.00% |
| 4 | 11.814 | 7.48515 | 47.70% |
| 5 | 12.671 | 6.98031 | 30.80% |
| 6 | 13.236 | 6.68362 | 8.30% |
| 7 | 14.496 | 6.1056 | 6.30% |
| 8 | 15.289 | 5.79046 | 8.90% |
| 9 | 16.063 | 5.51314 | 86.70% |
| 10 | 16.298 | 5.43443 | 74.80% |
| 11 | 16.867 | 5.25211 | 92.20% |
| 12 | 18.217 | 4.86602 | 33.50% |
| 13 | 18.783 | 4.72062 | 20.00% |
| 14 | 19.425 | 4.56604 | 16.80% |
| 15 | 20.114 | 4.41113 | 14.70% |
| 16 | 20.466 | 4.33592 | 22.50% |
| 17 | 22.615 | 3.92858 | 100.00% |
| 18 | 22.671 | 3.91903 | 98.90% |
| 19 | 23.085 | 3.8496 | 42.20% |
| 20 | 23.663 | 3.75691 | 12.10% |
| 21 | 24.099 | 3.68996 | 17.40% |
| 22 | 24.456 | 3.63683 | 17.00% |
| 23 | 25.965 | 3.42881 | 2.50% |
| 24 | 29.822 | 2.99358 | 3.50% |

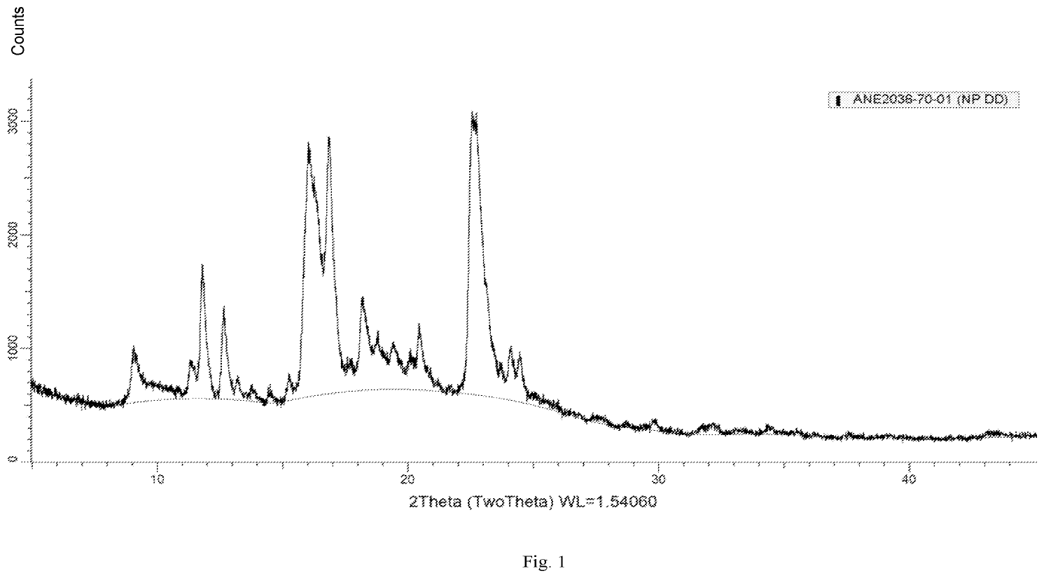

1.17. Any form of Salt 1.13-1.16, in the form of a crystal having an X-ray powder diffraction pattern corresponding to FIG. 1, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 1 generated using an X-ray diffractometer with a copper anode and a nickel filter.
1.18. Any form of Salt 1.13-1.17, in the form of a crystal having an X-ray powder diffraction pattern having at least 5, or at least 6, or at least 7, or at least 8, peaks having angle (2-theta) values selected from the group consisting of about 9.09, 9.19, 11.37, 11.81, 12.67, 13.24, 14.50, 15.29, 16.06, 16.30, 16.87, 18.22, 18.78, 19.43, 20.11, 20.47, 22.62, 22.67, 23.09, 23.66, 24.10, 24.46, 25.97, and 29.82, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
1.19. Any form of Salt 1.13-1.18, in the form of a crystal having an X-ray powder diffraction pattern having at least 5, or at least 6, or at least 7, or at least 8, peaks having d-spacing values selected from the group consisting of about 9.72, 9.61, 7.78, 7.49, 6.98, 6.68, 6.11, 5.79, 5.51, 5.43, 5.25, 4.87, 4.72, 4.57, 4.41, 4.34, 3.93, 3.92, 3.85, 3.76, 3.69, 3.64, 3.43, and 2.99, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
1.20. Any form of Salt 1.13-1.19, in the form of a crystal having an X-ray powder diffraction pattern having at least 5, or at least 6, or at least 7, or at least 8, peaks having angle (2-theta) values and/or d-spacing values as provided in 1.18 and 1.19.
1.21. Any foregoing form of Salt 1, wherein the lumateperone is deuterated, e.g., wherein the deuterium:protium ratio at one or more specified positions in the molecule is significantly higher, e.g., at least 2×, for example at least 10× higher, than the natural isotope ratios or the isotope ratios at other positions in the molecule; for example, any foregoing form of Salt 1 wherein either or both of the —CH₂— moieties of the piperazine ring are deuterated, e.g., —CHD- or —CD₂-, at levels which are significantly higher than the natural deuterium:protium isotope ratio or the deuterium:protium isotope ratio at other positions in the molecule, and/or wherein the methyl group on the piperazine ring is deuterated, e.g., is CD₃-, e.g., at levels which are significantly higher than the natural deuterium:protium isotope ratio or the deuterium:protium isotope ratio at other positions in the molecule; for example, wherein the deuterated lumateperone is any deuterated lumateperone as described in US 2019/0231780 or US 2021/0008065, the contents of each of which are hereby incorporated by reference in their entireties.
1.22. Any foregoing form of Salt 1 exhibiting any combination of characteristics as described in 1.1-1.21.

In a second embodiment, the present disclosure provides lumateperone in the form of a 4-tert-butylbenzenesulfonic acid addition salt (Salt 2). In further embodiments of Salt 2, the present disclosure provides:
2.1 Salt 2 in solid form.
2.2 Salt 2 or 2.1 in crystalline form, e.g., dry crystalline form.
2.3 Salt 2, 2.1, or 2.2, wherein the salt has about a 1:1 molar ratio of lumateperone to 4-tert-butylbenzenesulfonic acid (i.e., a mono-4-tert-butylbenzenesulfonate salt).
2.4 Salt 2, 2.1 or 2.2, wherein the salt has about a 1:2 molar ratio of lumateperone to 4-tert-butylbenzenesulfonic acid (i.e., a bis-4-tert-butylbenzenesulfonate salt).
2.5 Any foregoing form of Salt 2 which is a solvate, e.g., an ethyl acetate solvate.
2.6 Any foregoing form of Salt 2 which is not a solvate.
2.7 Any foregoing form of Salt 2 which is a hydrate.
2.8 Any foregoing form of Salt 2 which is not a hydrate.
2.9 Any foregoing form of Salt 2 formed by combining lumateperone free base and 4-tert-butylbenzenesulfonic acid in a molar ratio from 1:0.5 to 1:3, e.g., a 1:0.75 to 1:1.5 molar ratio, or a 1:0.75 to 1.25 molar ratio, or a 1:1.5 to 1:2.5 molar ratio, or a 1:1.75 to 1:2.25 molar ratio, or a 1:1.75 to 1:3 molar ratio, or a 1:1 to 1:2 molar ratio, or about a 1:1 molar ratio, or about a 1:1.5, or about a 1:2 molar ratio, or about a 1:2.5 molar ratio.
2.10 Any foregoing form of Salt 2 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. %, of amorphous forms.
2.11 Any foregoing form of Salt 2 in crystalline form, when crystallized from a mixture of 4-tert-butylbenzenesulfonic acid and lumateperone free base, e.g., in an organic solvent, e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof; e.g., ethyl acetate or toluene, optionally wherein the lumateperone free base and the 4-tert-butylbenzenesulfonic acid are in a molar ratio of about 1:2.
2.12 Salt 2.11, wherein the salt is crystallized from the solvent after an anti-solvent is added, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane.
2.13 Any foregoing form of Salt 2, wherein the salt is formed from a 1:2 molar ratio of lumateperone free base to 4-tert-butylbenzenesulfonic acid in ethyl acetate solvent.
2.14 Salt 2.13, wherein a proton-NMR analysis of the salt shows a molar ratio of lumateperone to 4-tert-butylbenzenesulfonic acid of about 1:2.
2.15 Salt 2.13 or 2.14, wherein a DSC analysis shows one endothermic event at about 68° C. (e.g., a desolvation), and one endothermic event at about 212° C. (e.g., a melt).
2.16 Any form of Salt 2.13-2.15, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 2, 4, 5, 6, 7, 9, 11, and/or 12:

XRPD (Cu anode, Ni filter) for 4-tert-
Butylbenzenesulfonate Salt Crystal

| # | Angle | d Value | Rel. Intensity |
|---|---|---|---|
| 1 | 3.216 | 27.44733 | 13.00% |
| 2 | 3.640 | 24.25154 | 100.00% |
| 3 | 6.665 | 13.25211 | 12.90% |
| 4 | 7.194 | 12.27861 | 20.60% |
| 5 | 13.904 | 6.36395 | 14.90% |
| 6 | 14.724 | 6.01133 | 12.70% |
| 7 | 15.224 | 5.81524 | 14.30% |
| 8 | 15.642 | 5.66062 | 10.10% |
| 9 | 16.050 | 5.51782 | 12.60% |
| 10 | 17.955 | 4.93645 | 8.10% |
| 11 | 18.775 | 4.72263 | 41.10% |
| 12 | 18.903 | 4.69091 | 24.20% |
| 13 | 19.586 | 4.52885 | 17.00% |
| 14 | 20.657 | 4.29632 | 13.60% |
| 15 | 22.084 | 4.02185 | 7.30% |
| 16 | 22.994 | 3.86463 | 5.20% |
| 17 | 23.678 | 3.75456 | 10.20% |

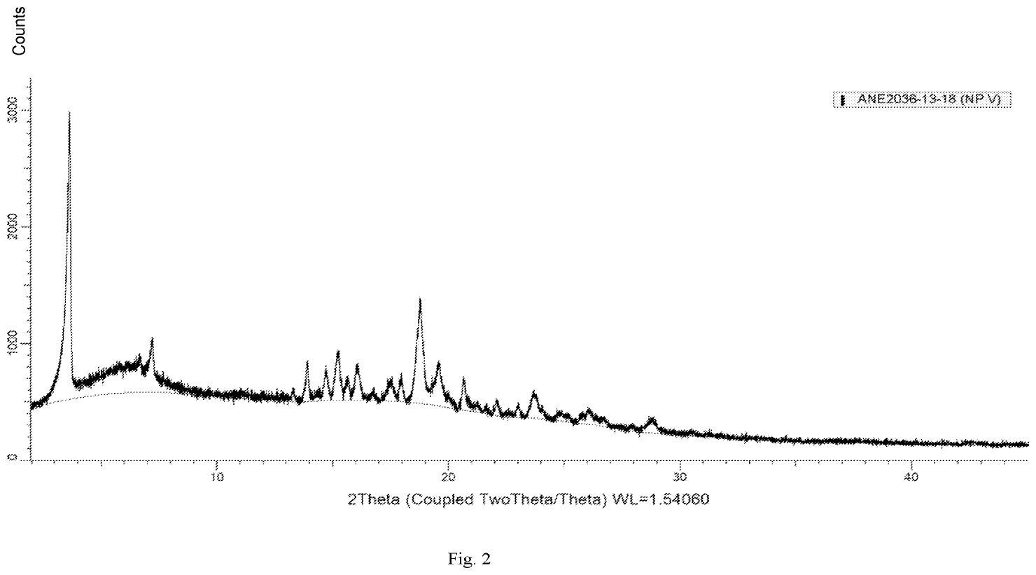

2.17 Any form of Salt 2.13-2.16, in the form of a crystal having an X-ray powder diffraction pattern corresponding to FIG. 2, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 2 generated using an X-ray diffractometer with a copper anode and a nickel filter.
2.18 Any form of Salt 2.13-2.17, in the form of a crystal having an X-ray powder diffraction pattern having at least 5, or at least 6, or at least 7, or at least 8, peaks having angle (2-theta) values selected from the group consisting of about 3.22, 3.64, 6.67, 7.19, 13.90, 14.72, 15.22, 15.64, 16.05, 17.96, 18.78, 18.90, 19.59, 20.66, 22.08, 22.99, and 23.68, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
2.19 Any form of Salt 2.13-2.18, in the form of a crystal having an X-ray powder diffraction pattern having at least 5, or at least 6, or at least 7, or at least 8, peaks having d-spacing values selected from the group consisting of about 27.45, 24.25, 13.25, 12.28, 6.36, 6.01, 5.82, 5.66, 5.52, 4.94, 4.72, 4.69, 4.53, 4.30, 4.02, 3.86, and 3.75, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
2.20 Any form of Salt 2.13-2.19, in the form of a crystal having an X-ray powder diffraction pattern having at least 5, or at least 6, or at least 7, or at least 8, peaks having angle (2-theta) values and/or d-spacing values as provided in 2.18 and 2.19.
2.21 Any foregoing form of Salt 2, wherein the lumateperone is deuterated, e.g., wherein the deuterium:protium ratio at one or more specified positions in the molecule is significantly higher, e.g., at least 2×, for example at least 10× higher, than the natural isotope ratios or the isotope ratios at other positions in the molecule; for example, any foregoing form of Salt 1 wherein either or both of the —CH₂— moieties of the piperazine ring are deuterated, e.g., —CHD- or —CD₂-, at levels which are significantly higher than the natural deuterium:protium isotope ratio or the deuterium:protium isotope ratio at other positions in the molecule, and/or wherein the methyl group on the piperazine ring is deuterated, e.g., is CD₃-, e.g., at levels which are significantly higher than the natural deuterium:protium isotope ratio or the deuterium:protium isotope ratio at other positions in the molecule; for example, wherein the deuterated lumateperone is any deuterated lumateperone as described in US 2019/0231780 or US 2021/0008065, the contents of each of which are hereby incorporated by reference in their entireties.
2.22 Any foregoing form of Salt 2 exhibiting any combination of characteristics as described in 2.1-2.21.

In a third embodiment, the present disclosure provides lumateperone in the form of a 4-propyllbenzenesulfonic acid addition salt (Salt 3). In further embodiments of Salt 3, the present disclosure provides:
3.1 Salt 3 in solid form.
3.2 Salt 3 or 3.1 in crystalline form, e.g., dry crystalline form.
3.3 Salt 3, 3.1, or 3.2, wherein the salt has about a 1:1 molar ratio of lumateperone to 4-propylbenzenesulfonic acid (i.e., a mono-4-propylbenzenesulfonate salt).
3.4 Salt 3, 3.1, or 3.2, wherein the salt has about a 1:2 molar ratio of lumateperone to 4-propylbenzenesulfonic acid (i.e., a bis-4-propylbenzenesulfonate salt).
3.5 Any foregoing form of Salt 3 which is a solvate, e.g., an ethyl acetate solvate.
3.6 Any foregoing form of Salt 3 which is not a solvate.
3.7 Any foregoing form of Salt 3 which is a hydrate.
3.8 Any foregoing form of Salt 3 which is not a hydrate.
3.9 Any foregoing form of Salt 3 formed by combining lumateperone free base and 4-propylbenzenesulfonic acid in a molar ratio from 1:0.5 to 1:3, e.g., a 1:0.75 to 1:1.5 molar ratio, or a 1:0.75 to 1.25 molar ratio, or a 1:1.5 to 1:2.5 molar ratio, or a 1:1.75 to 1:2.25 molar ratio, or a 1:1.75 to 1:3 molar ratio, or a 1:1 to 1:2 molar ratio, or about a 1:1 molar ratio, or about a 1:1.5, or about a 1:2 molar ratio, or about a 1:2.5 molar ratio.
3.10 Any foregoing form of Salt 3 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. %, of amorphous forms.
3.11 Any foregoing form of Salt 3 in crystalline form, when crystallized from a mixture of 4-propylbenzenesulfonic acid and lumateperone free base, e.g., in an organic solvent, e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof; e.g., ethyl acetate, optionally wherein the lumateperone free base and the 4-propylbenzenesulfonic acid are in a molar ratio of about 1:2.
3.12 Salt 3.11, wherein the salt is crystallized from the solvent after an anti-solvent is added, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane.
3.13 Any foregoing form of Salt 3, wherein the salt is formed from a 1:2 molar ratio of lumateperone free base to 4-propylbenzenesulfonic acid in ethyl acetate solvent.
3.14 Salt 3.13, wherein a proton-NMR analysis of the salt shows a molar ratio of lumateperone to 4-propylbenzenesulfonic acid of about 1:2.
3.15 Salt 3.13 or 3.14, wherein a DSC analysis shows one endothermic event at about 159° C. (e.g., a melt).
3.16 Any form of Salt 3.13-3.15, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 1, 5, 9, 10, 14, and/or 18:

XRPD (Cu anode, Ni filter) for 4-Propylbenzenesulfonate
Salt Crystal

| # | Angle | d Value | Rel. Intensity |
|---|---|---|---|
| 1 | 4.049 | 21.80405 | 100.00% |
| 2 | 8.007 | 11.03315 | 7.60% |
| 3 | 10.546 | 8.38185 | 6.20% |
| 4 | 12.624 | 7.00636 | 3.70% |
| 5 | 13.049 | 6.77933 | 9.60% |
| 6 | 13.644 | 6.48472 | 4.30% |
| 7 | 14.380 | 6.15464 | 3.10% |
| 8 | 14.804 | 5.97913 | 2.20% |
| 9 | 15.308 | 5.78332 | 12.30% |
| 10 | 16.458 | 5.38181 | 12.90% |
| 11 | 17.330 | 5.11302 | 5.30% |
| 12 | 17.754 | 4.99186 | 4.90% |
| 13 | 19.126 | 4.63669 | 4.60% |
| 14 | 19.984 | 4.43940 | 7.70% |
| 15 | 20.443 | 4.34085 | 7.30% |
| 16 | 20.751 | 4.27719 | 2.80% |
| 17 | 21.111 | 4.20488 | 7.00% |
| 18 | 21.451 | 4.13904 | 17.90% |
| 19 | 21.952 | 4.04574 | 2.80% |
| 20 | 22.284 | 3.98629 | 5.90% |
| 21 | 22.859 | 3.88719 | 2.00% |
| 22 | 23.652 | 3.75860 | 3.80% |
| 23 | 24.076 | 3.69338 | 1.70% |
| 24 | 25.611 | 3.47542 | 2.20% |
| 25 | 26.257 | 3.39137 | 4.40% |
| 26 | 26.824 | 3.32095 | 1.80% |
| 27 | 28.067 | 3.17665 | 2.40% |
| 28 | 31.375 | 2.84884 | 1.40% |

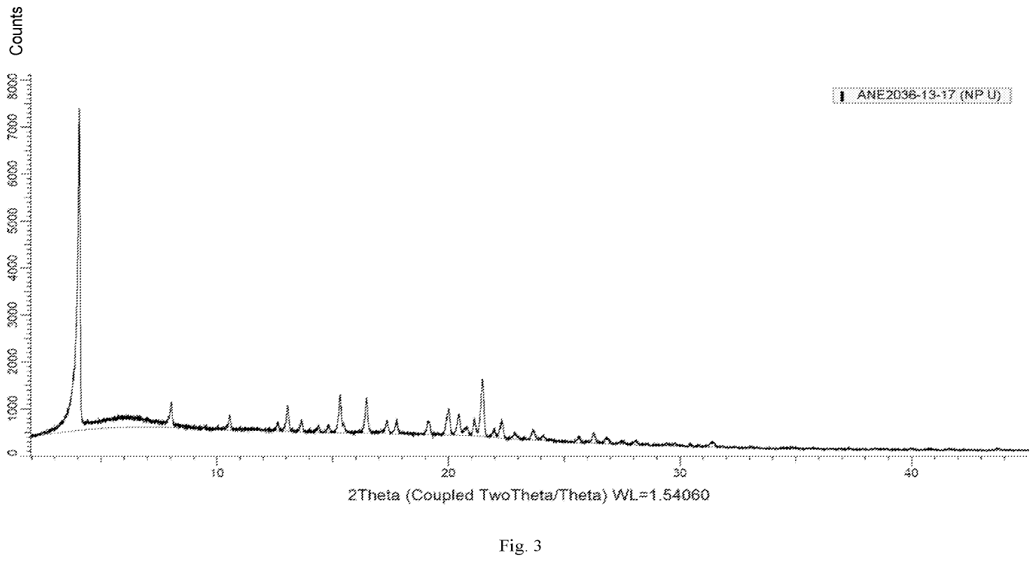

3.17 Any form of Salt 3.13-3.16, in the form of a crystal having an X-ray powder diffraction pattern corresponding to FIG. 3, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 3 generated using an X-ray diffractometer with a copper anode and a nickel filter.
3.18 Any form of Salt 3.13-3.17, in the form of a crystal having an X-ray powder diffraction pattern having at least 5, or at least 6, or at least 7, or at least 8, peaks having angle (2-theta) values selected from the group consisting of about 4.05, 8.01, 10.55, 12.62, 13.05, 13.64, 14.38, 14.80, 15.31, 16.46, 17.33, 17.75, 19.13, 19.98, 20.44, 20.75, 21.11, 21.45, 21.95, 22.28, 22.86, 23.65, 24.08, 25.61, 26.26, 26.82, 28.07, and 31.38, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
3.19 Any form of Salt 3.13-3.18, in the form of a crystal having an X-ray powder diffraction pattern having at least 5, or at least 6, or at least 7, or at least 8, peaks having d-spacing values selected from the group consisting of about 21.80, 11.03, 8.38, 7.01, 6.78, 6.48, 6.15, 5.98, 5.78, 5.38, 5.11, 4.99, 4.64, 4.44, 4.34, 4.28, 4.20, 4.14, 4.05, 3.99, 3.89, 3.76, 3.69, 3.48, 3.39, 3.32, 3.18, and 2.85, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
3.20 Any form of Salt 3.13-3.19, in the form of a crystal having an X-ray powder diffraction pattern having at least 5, or at least 6, or at least 7, or at least 8, peaks having angle (2-theta) values and/or d-spacing values as provided in 3.18 and 3.19.
3.21 Any foregoing form of Salt 3 wherein the lumateperone is deuterated, e.g., wherein the deuterium:protium ratio at one or more specified positions in the molecule is significantly higher, e.g., at least 2×, for example at least 10× higher, than the natural isotope ratios or the isotope ratios at other positions in the molecule; for example, any foregoing form of Salt 1 wherein either or both of the —CH₂— moieties of the piperazine ring are deuterated, e.g., —CHD- or —CD₂-, at levels which are significantly higher than the natural deuterium:protium isotope ratio or the deuterium:protium isotope ratio at other positions in the molecule, and/or wherein the methyl group on the piperazine ring is deuterated, e.g., is CD₃-, e.g., at levels which are significantly higher than the natural deuterium:protium isotope ratio or the deuterium:protium isotope ratio at other positions in the molecule; for example, wherein the deuterated lumateperone is any deuterated lumateperone as described in US 2019/0231780 or US 2021/0008065, the contents of each of which are hereby incorporated by reference in their entireties.
3.22 Any foregoing form of Salt 3 exhibiting any combination of characteristics as described in 3.1-3.21.

In a fourth embodiment, the present disclosure provides lumateperone in the form of a 4-ethylbenzenesulfonic acid addition salt (Salt 4). In further embodiments of Salt 4, the present disclosure provides:
4.1 Salt 4 in solid form.
4.2 Salt 4, or 4.1 in crystalline form, e.g., dry crystalline form.
4.3 Salt 4, 4.1, or 4.2, wherein the salt has a 1:1 molar ratio of lumateperone to 4-ethylbenzenesulfonic acid (i.e., a mono-4-ethylbenzenesulfonate salt).
4.4 Salt 4, 4.1, or 4.2, wherein the salt has a 1:2 molar ratio of lumateperone to 4-ethylbenzenesulfonic acid (i.e., a bis-4-ethylbenzenesulfonate salt).
4.5 Any foregoing form of Salt 4 which is a solvate, e.g., an ethyl acetate solvate, or a toluene solvate.
4.6 Any foregoing form of Salt 4 which is not a solvate.
4.7 Any foregoing form of Salt 4 which is a hydrate.
4.8 Any foregoing form of Salt 4 which is not a hydrate.
4.9 Any foregoing form of Salt 4 formed by combining lumateperone free base and 4-ethylbenzenesulfonic acid in a molar ratio from 1:0.5 to 1:3, e.g., a 1:0.75 to 1:1.5 molar ratio, or a 1:0.75 to 1.25 molar ratio, or a 1:1.5 to 1:2.5 molar ratio, or a 1:1.75 to 1:2.25 molar ratio, or a 1:1.75 to 1:3 molar ratio, or a 1:1 to 1:2 molar ratio, or about a 1:1 molar ratio, or about a 1:1.5, or about a 1:2 molar ratio, or about a 1:2.5 molar ratio.
4.10 Any foregoing form of Salt 4 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. %, of amorphous forms.
4.11 Any foregoing form of Salt 1 in crystalline form, when crystallized from a mixture of 4-ethylbenzenesulfonic acid and lumateperone free base, e.g., in an organic solvent, e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof; e.g., ethyl acetate or toluene, optionally wherein the lumateperone free base and the 4-ethylbenzenesulfonic acid are in a molar ratio of about 1:1 or about 1:2.
4.12 Salt 4.11, wherein the salt is crystallized from the solvent after an anti-solvent is added, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane, e.g., wherein the organic solvent is toluene and the anti-solvent is heptane.
4.13 Any of Salts 4 or 4.1-4.12, wherein the salt is formed from a 1:2 molar ratio of lumateperone free base to 4-ethylbenzenesulfonic acid in ethyl acetate solvent.
4.14 Salt 4.13, wherein a proton-NMR analysis of the salt shows a molar ratio of lumateperone to 4-ethylbenzenesulfonic acid of about 1:2.
4.15 Salt 4.13 or 4.14, wherein a DSC analysis shows one endothermic event at about 147° C. (e.g., a melt), or one endothermic event at about 156° C. (e.g., a melt), or one endothermic event at about 138° C. (e.g., a melt).
4.16 Any form of Salt 4.13-4.15, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 1, 2, 3, 10, 11, 14, 15, and/or 16:

XRPD (Cu anode, Ni filter) for 4-Ethylbenzenesulfonate
Salt Crystal Polymorph 1

| # | Angle | d Value | Rel. Intensity |
|---|---|---|---|
| 1 | 3.511 | 25.14320 | 100.00% |
| 2 | 3.684 | 23.96635 | 55.30% |
| 3 | 4.982 | 17.72257 | 14.80% |
| 4 | 6.917 | 12.76937 | 5.60% |
| 5 | 7.321 | 12.06545 | 5.30% |
| 6 | 10.354 | 8.53641 | 2.60% |
| 7 | 10.383 | 8.51268 | 2.80% |
| 8 | 12.488 | 7.08228 | 2.60% |
| 9 | 13.689 | 6.46357 | 4.10% |
| 10 | 14.269 | 6.20219 | 9.00% |
| 11 | 15.587 | 5.68057 | 40.60% |
| 12 | 17.499 | 5.06386 | 9.70% |
| 13 | 17.682 | 5.01186 | 8.80% |
| 14 | 18.581 | 4.77134 | 13.70% |
| 15 | 18.852 | 4.70347 | 13.50% |
| 16 | 20.127 | 4.40823 | 27.20% |
| 17 | 20.609 | 4.30628 | 13.50% |
| 18 | 20.833 | 4.26051 | 12.20% |
| 19 | 21.246 | 4.17848 | 7.60% |
| 20 | 22.016 | 4.03418 | 5.30% |
| 21 | 27.486 | 3.24248 | 4.50% |

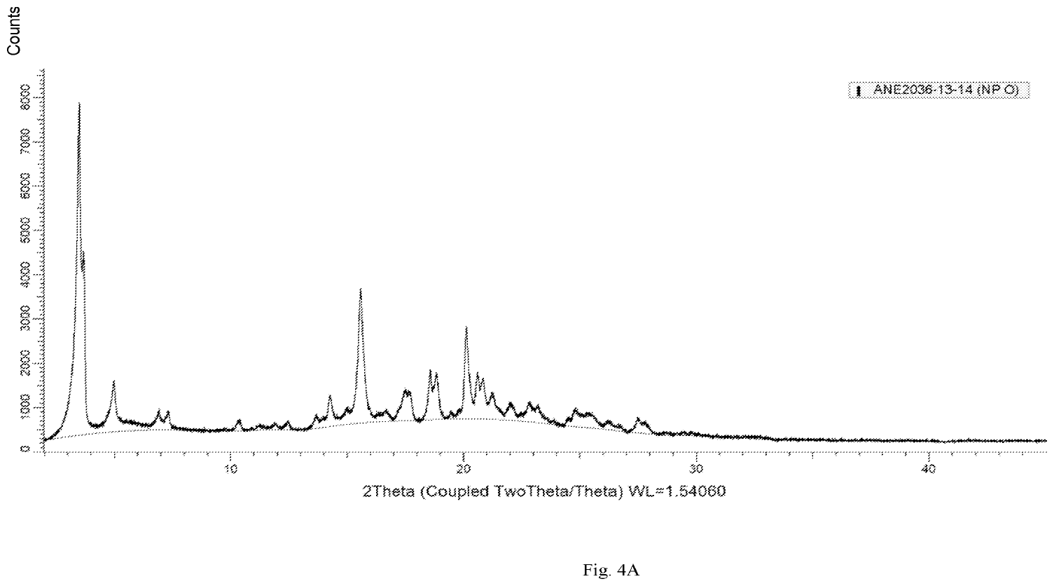

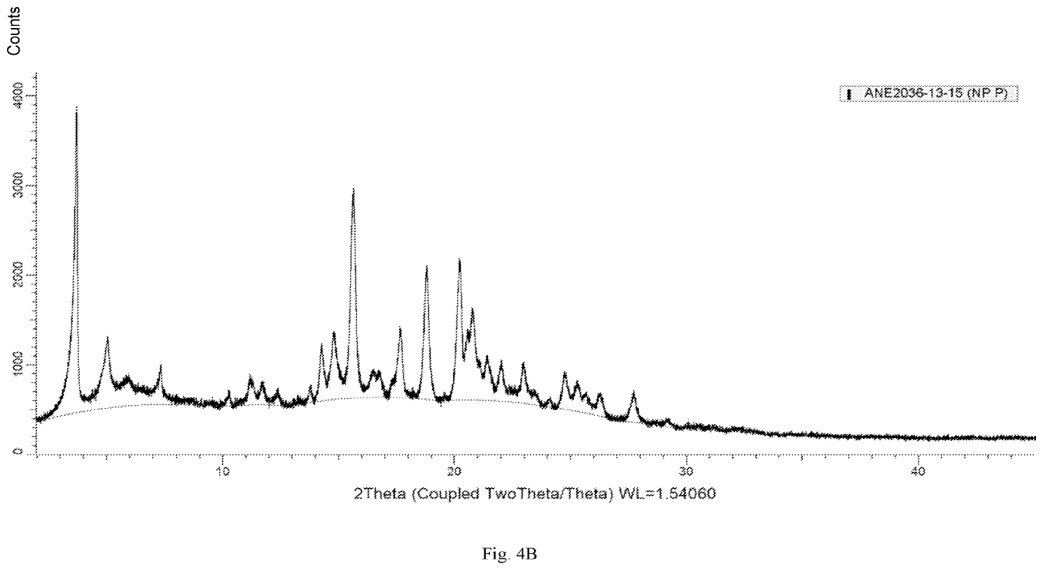

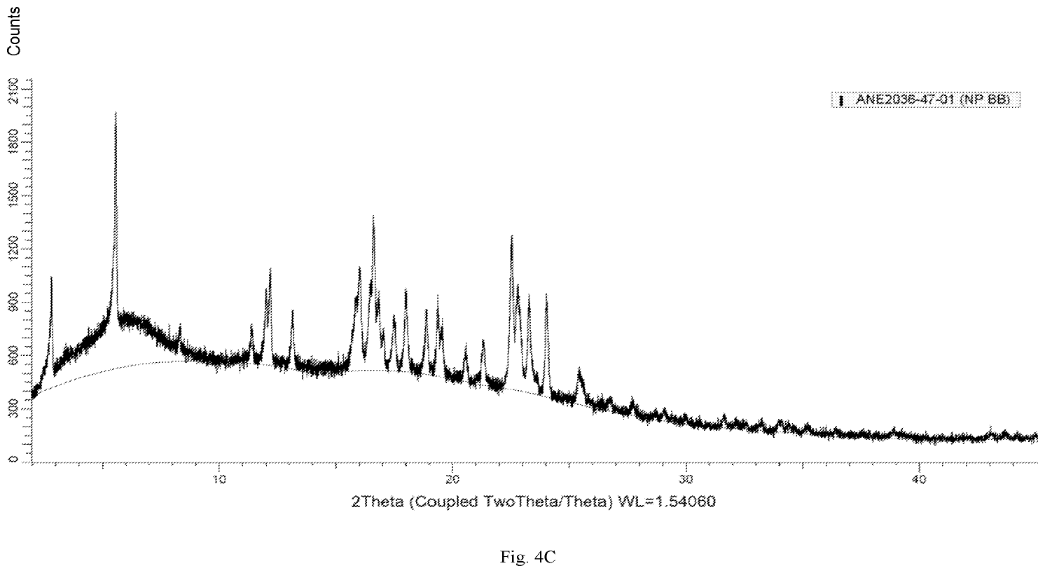

4.17 Any form of Salt 4.13-4.16, in the form of a crystal having an X-ray powder diffraction pattern corresponding to FIG. 4A, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 4A generated using an X-ray diffractometer with a copper anode and a nickel filter.
4.18 Any form of Salt 4.13-4.17, in the form of a crystal having an X-ray powder diffraction pattern having at least 5, or at least 6, or at least 7, or at least 8, peaks having angle (2-theta) values selected from the group consisting of about 3.51, 3.68, 4.98, 6.92, 7.32, 10.35, 10.38, 12.49, 13.69, 14.27, 15.59, 17.50, 17.68, 18.58, 18.85, 20.13, 20.61, 20.83, 21.25, 22.02, and 27.49, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
4.19 Any form of Salt 4.13-4.18, in the form of a crystal having an X-ray powder diffraction pattern having at least 5, or at least 6, or at least 7, or at least 8, peaks having d-spacing values selected from the group consisting of about 25.14, 23.97, 17.72, 12.77, 12.07, 8.54, 8.51, 7.08, 6.46, 6.20, 5.68, 5.06, 5.01, 4.77, 4.70, 4.41, 4.31, 4.26, 4.18, 4.03, and 3.24, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
4.20 Any form of Salt 4.13-4.19, in the form of a crystal having an X-ray powder diffraction pattern having at least 5, or at least 6, or at least 7, or at least 8, peaks having angle (2-theta) values and/or d-spacing values as provided in 4.18 and 4.19.
4.21 Any of Salts 4 or 4.1-4.12, wherein the salt is formed from a 1:2 molar ratio of lumateperone free base to 4-ethylbenzenesulfonic acid in toluene solvent.
4.22 Salt 4.21, wherein a proton-NMR analysis of the salt shows a molar ratio of lumateperone to 4-ethylbenzenesulfonic acid of about 1:2.
4.23 Salt 4.21 or 4.22, wherein a DSC analysis shows one endothermic event at about 156° C. (e.g., a melt), or one endothermic event at about 147° C. (e.g., a melt), or one endothermic event at about 138° C. (e.g., a melt).
4.24 Any form of Salt 4.21-4.23, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 1, 2, 3, 8, 9, 10, 12, 13, 14 and/or 16:

XRPD (Cu anode, Ni filter) for 4-Ethylbenzenesulfonate
Salt Crystal Polymorph 2

| # | Angle | d Value | Rel. Intensity |
|---|---|---|---|
| 1 | 3.694 | 23.89639 | 100.00% |
| 2 | 5.034 | 17.54009 | 24.00% |
| 3 | 7.319 | 12.06926 | 13.00% |
| 4 | 10.267 | 8.60911 | 6.10% |
| 5 | 11.190 | 7.90062 | 0.30% |
| 6 | 11.695 | 7.56092 | 7.00% |
| 7 | 13.798 | 6.41287 | 6.70% |
| 8 | 14.271 | 6.20112 | 16.40% |
| 9 | 14.794 | 5.98337 | 24.30% |
| 10 | 15.630 | 5.66494 | 74.70% |
| 11 | 16.492 | 5.37089 | 11.00% |
| 12 | 17.661 | 5.01793 | 27.40% |
| 13 | 18.800 | 4.71645 | 50.80% |
| 14 | 20.221 | 4.38793 | 53.10% |
| 15 | 20.575 | 4.31324 | 25.80% |
| 16 | 20.782 | 4.27078 | 34.30% |
| 17 | 21.075 | 4.21203 | 14.90% |
| 18 | 21.413 | 4.14627 | 16.10% |
| 19 | 22.019 | 4.03362 | 14.10% |
| 20 | 22.956 | 3.87095 | 14.00% |
| 21 | 24.085 | 3.69203 | 4.60% |
| 22 | 24.743 | 3.59528 | 13.20% |
| 23 | 25.281 | 3.52006 | 12.00% |
| 24 | 26.244 | 3.39300 | 0.90% |
| 25 | 27.703 | 3.21750 | 11.40% |

4.25 Any form of Salt 4.21-4.24, in the form of a crystal having an X-ray powder diffraction pattern corresponding to FIG. 4B, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 4B generated using an X-ray diffractometer with a copper anode and a nickel filter.
4.26 Any form of Salt 4.21-4.25, in the form of a crystal having an X-ray powder diffraction pattern having at least 5, or at least 6, or at least 7, or at least 8, peaks having angle (2-theta) values selected from the group consisting of about 3.69, 5.03, 7.32, 10.27, 11.19, 11.70, 13.80, 14.27, 14.79, 15.63, 16.49, 17.66, 18.80, 20.22, 20.58, 20.78, 21.08, 21.41, 22.02, 22.96, 24.09, 24.74, 25.28, 26.24, and 27.70, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
4.27 Any form of Salt 4.21-4.26, in the form of a crystal having an X-ray powder diffraction pattern having at least 5, or at least 6, or at least 7, or at least 8, peaks having d-spacing values selected from the group consisting of about 23.90, 17.54, 12.07, 8.61, 7.90, 7.56, 6.41, 6.20, 5.98, 5.66, 5.37, 5.02, 4.72, 4.39, 4.31, 4.27, 4.21, 4.15, 4.03, 3.87, 3.69, 3.60, 3.52, 3.39, and 3.22, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
4.28 Any form of Salt 4.21-4.27, in the form of a crystal having an X-ray powder diffraction pattern having at least 5, or at least 6, or at least 7, or at least 8, peaks having angle (2-theta) values and/or d-spacing values as provided in 4.26 and 4.27.
4.29 Any of Salts 4 or 4.1-4.12, wherein the salt is formed from a 1:1 molar ratio of lumateperone free base to 4-ethylbenzenesulfonic acid in toluene solvent with heptane anti-solvent.
4.30 Salt 4.29, wherein a proton-NMR analysis of the salt shows a molar ratio of lumateperone to 4-ethylbenzenesulfonic acid of about 1:1.
4.31 Salt 4.29 or 4.30, wherein a DSC analysis shows one endothermic event at about 138° C. (e.g., a melt), or one endothermic event at about 156° C. (e.g., a melt), or one endothermic event at about 147° C. (e.g., a melt).
4.32 Any form of Salt 4.29-4.31, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 2, 4, 5, 6, 7, 9, 14, 16, and/or 17:

XRPD (Cu anode, Ni filter) for 4-Ethylbenzenesulfonate
Salt Crystal Polymorph 3

| # | Angle | d Value | Rel. Intensity |
|---|---|---|---|
| 1 | 2.789 | 31.65519 | 30.70% |
| 2 | 5.569 | 15.85574 | 100.00% |
| 3 | 11.382 | 7.76773 | 16.90% |
| 4 | 12.090 | 7.31483 | 19.80% |
| 5 | 13.121 | 6.74217 | 21.00% |
| 6 | 15.965 | 5.54681 | 28.90% |
| 7 | 16.585 | 5.34081 | 57.90% |
| 8 | 17.487 | 5.06740 | 24.40% |
| 9 | 17.996 | 4.92527 | 27.00% |
| 10 | 18.865 | 4.70026 | 26.20% |
| 11 | 19.406 | 4.57029 | 21.80% |
| 12 | 20.542 | 4.32010 | 11.20% |
| 13 | 21.306 | 4.16702 | 20.10% |
| 14 | 22.536 | 3.94215 | 63.20% |
| 15 | 22.796 | 3.89784 | 40.40% |
| 16 | 23.272 | 3.81916 | 36.00% |
| 17 | 24.020 | 3.70193 | 50.10% |
| 18 | 25.453 | 3.49666 | 15.10% |
| 19 | 31.614 | 2.82786 | 5.90% |
| 20 | 34.006 | 2.63421 | 3.50% |

4.33 Any form of Salt 4.29-4.32, in the form of a crystal having an X-ray powder diffraction pattern corresponding to FIG. 4C, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 4C generated using an X-ray diffractometer with a copper anode and a nickel filter.
4.34 Any form of Salt 4.29-4.33, in the form of a crystal having an X-ray powder diffraction pattern having at least 5, or at least 6, or at least 7, or at least 8, peaks having angle (2-theta) values selected from the group consisting of about 2.79, 5.57, 11.38, 12.09, 13.12, 15.97, 16.59, 17.49, 18.00, 18.87, 19.41, 20.54, 21.31, 22.54, 22.80, 23.27, 24.02, 25.45, 31.61, and 34.01, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
4.35 Any form of Salt 4.29-4.34, in the form of a crystal having an X-ray powder diffraction pattern having at least 5, or at least 6, or at least 7, or at least 8, peaks having d-spacing values selected from the group consisting of about 31.66, 15.86, 7.77, 7.31, 6.74, 5.55, 5.34, 5.07, 4.93, 4.70, 4.57, 4.32, 4.17, 3.94, 3.90, 3.82, 3.70, 3.50, 2.83, and 2.63, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
4.36 Any form of Salt 4.29-4.35, in the form of a crystal having an X-ray powder diffraction pattern having at least 5, or at least 6, or at least 7, or at least 8, peaks having angle (2-theta) values and/or d-spacing values as provided in 4.34 and 4.35.
4.37 Any foregoing form of Salt 4 wherein the lumateperone is deuterated, e.g., wherein the deuterium:protium ratio at one or more specified positions in the molecule is significantly higher, e.g., at least 2×, for example at least 10× higher, than the natural isotope ratios or the isotope ratios at other positions in the molecule; for example, any foregoing form of Salt 1 wherein either or both of the —CH₂— moieties of the piperazine ring are deuterated, e.g., —CHD- or —CD₂-, at levels which are significantly higher than the natural deuterium:protium isotope ratio or the deuterium:protium isotope ratio at other positions in the molecule, and/or wherein the methyl group on the piperazine ring is deuterated, e.g., is CD₃-, e.g., at levels which are significantly higher than the natural deuterium:protium isotope ratio or the deuterium:protium isotope ratio at other positions in the molecule; for example, wherein the deuterated lumateperone is any deuterated lumateperone as described in US 2019/0231780 or US 2021/0008065, the contents of each of which are hereby incorporated by reference in their entireties.
4.38 Any foregoing form of Salt 4 exhibiting any combination of characteristics as described in 4.1-4.37.

In a fifth embodiment, the present disclosure provides lumateperone in the form of a 2-naphthalenesulfonic acid addition salt, wherein the salt is a solvate (Salt 5). In further embodiments of Salt 5, the present disclosure provides:
5.1 Salt 5 in solid form.
5.2 Salt 5 or 5.1 in crystalline form, e.g., dry crystalline form.
5.3 Salt 5, 5.1, or 5.2, wherein the salt has a 1:1 molar ratio of lumateperone to 2-naphthalenesulfonic acid (i.e., a mono-napsylate salt).
5.4 Salt 5, 5.1, or 5.2, wherein the salt has a 1:2 molar ratio of lumateperone to 2-naphthalenesulfonic acid (i.e., a bis-napsylate salt).
5.5 Any foregoing form of Salt 5, wherein the salt is an ethyl acetate solvate.
5.6 Any foregoing form of Salt 5 which is a hydrate.
5.7 Any foregoing form of Salt 5 which is not a hydrate.
5.8 Any foregoing form of Salt 5 formed by combining lumateperone free base and 2-naphthalenesulfonic acid in a molar ratio from 1:0.5 to 1:3, e.g., a 1:0.75 to 1:1.5 molar ratio, or a 1:0.75 to 1.25 molar ratio, or a 1:1.5 to 1:2.5 molar ratio, or a 1:1.75 to 1:2.25 molar ratio, or a 1:1.75 to 1:3 molar ratio, or a 1:1 to 1:2 molar ratio, or about a 1:1 molar ratio, or about a 1:1.5, or about a 1:2 molar ratio, or about a 1:2.5 molar ratio.
5.9 Any foregoing form of Salt 5, in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. %, of amorphous forms.
5.10 Any foregoing form of Salt 5 in crystalline form, when crystallized from a mixture of 2-naphthalenesulfonic acid and lumateperone free base, e.g., in an organic solvent, e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof; e.g., ethyl acetate, optionally wherein the lumateperone free base and the 2-naphthalenesulfonic acid are in a molar ratio of about 1:2.
5.11 Salt 5.10, wherein the salt is crystallized from the solvent after an anti-solvent is added, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane.
5.12 Any foregoing form of Salt 5, wherein the salt is formed from a 1:2 molar ratio of lumateperone free base to 2-naphthalenesulfonic acid in ethyl acetate solvent.
5.13 Salt 5.12, wherein a proton-NMR analysis of the salt shows a molar ratio of lumateperone to 2-naphthalenesulfonic acid of about 1:2.
5.14 Salt 5.12 or 5.13, wherein a DSC analysis shows one endothermic event at about 108° C. (e.g., a desolvation), and/or one endothermic event at about 162° C. (e.g., a melt).
5.15 Any form of Salt 5.12-5.14, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 1, 2, 3, 4, 5, 8, 9, 11, 14, and/or 16:

XRPD (Cu anode, Ni filter) for 2-
Naphthalenesulfonate Salt Crystal

| # | Angle | d Value | Rel. Intensity |
|---|---|---|---|
| 1 | 2.401 | 36.77034 | 32.50% |
| 2 | 7.045 | 12.53758 | 16.40% |
| 3 | 14.063 | 6.29268 | 10.20% |
| 4 | 14.906 | 5.93853 | 27.30% |
| 5 | 15.477 | 5.72064 | 26.40% |
| 6 | 17.204 | 5.15024 | 25.70% |
| 7 | 17.493 | 5.06578 | 21.00% |
| 8 | 19.018 | 4.66279 | 26.30% |
| 9 | 20.323 | 4.36625 | 40.90% |
| 10 | 20.418 | 4.34608 | 39.50% |
| 11 | 20.780 | 4.27126 | 84.70% |
| 12 | 20.842 | 4.25855 | 100.00% |
| 13 | 21.247 | 4.17835 | 28.40% |
| 14 | 23.846 | 3.72847 | 21.10% |
| 15 | 26.939 | 3.30704 | 9.30% |
| 16 | 27.208 | 3.27490 | 28.90% |

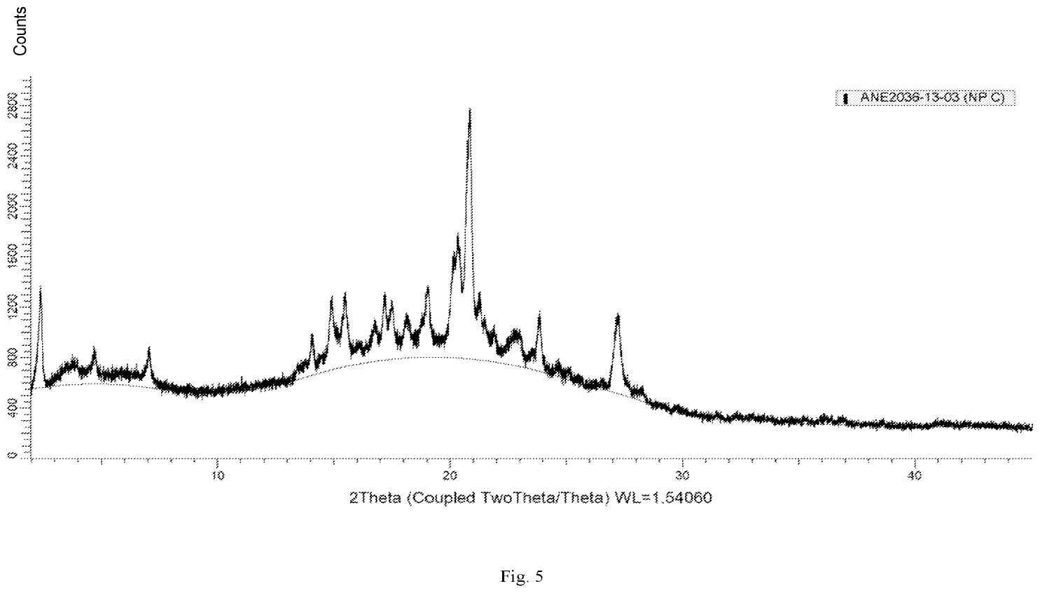

5.16 Any form of Salt 5.12-5.15, in the form of a crystal having an X-ray powder diffraction pattern corresponding to FIG. 5, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 5 generated using an X-ray diffractometer with a copper anode and a nickel filter.
5.17 Any form of Salt 5.12-5.16, in the form of a crystal having an X-ray powder diffraction pattern having at least 5, or at least 6, or at least 7, or at least 8, peaks having angle (2-theta) values selected from the group consisting of about 2.40, 7.05, 14.06, 14.91, 15.48, 17.20, 17.49, 19.02, 20.32, 20.42, 20.78, 20.84, 21.25, 23.85, 26.94, and 27.21, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
5.18 Any form of Salt 5.12-5.17, in the form of a crystal having an X-ray powder diffraction pattern having at least 5, or at least 6, or at least 7, or at least 8, peaks having d-spacing values selected from the group consisting of about 36.77, 12.54, 6.29, 5.94, 5.72, 5.15, 5.07, 4.66, 4.37, 4.35, 4.27, 4.26, 4.18, 3.73, 3.31, and 3.27, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
5.19 Any form of Salt 5.12-5.18, in the form of a crystal having an X-ray powder diffraction pattern having at least 5, or at least 6, or at least 7, or at least 8, peaks having angle (2-theta) values and/or d-spacing values as provided in 5.17 and 5.18.
5.20 Any foregoing form of Salt 5, wherein the lumateperone is deuterated, e.g., wherein the deuterium:protium ratio at one or more specified positions in the molecule is significantly higher, e.g., at least 2×, for example at least 10× higher, than the natural isotope ratios or the isotope ratios at other positions in the molecule; for example, any foregoing form of Salt 1 wherein either or both of the —CH₂— moieties of the piperazine ring are deuterated, e.g., —CHD- or —CD₂-, at levels which are significantly higher than the natural deuterium:protium isotope ratio or the deuterium:protium isotope ratio at other positions in the molecule, and/or wherein the methyl group on the piperazine ring is deuterated, e.g., is CD₃-, e.g., at levels which are significantly higher than the natural deuterium:protium isotope ratio or the deuterium:protium isotope ratio at other positions in the molecule; for example, wherein the deuterated lumateperone is any deuterated lumateperone as described in US 2019/0231780 or US 2021/0008065, the contents of each of which are hereby incorporated by reference in their entireties.
5.21 Any foregoing form of Salt 5 exhibiting any combination of characteristics as described in 5.1-5.20.

In a sixth embodiment, the present disclosure provides lumateperone in the form of a solid crystalline benzenesulfonic acid addition salt (Salt 6), wherein the salt is a crystal characterized by a DSC thermogram lacking an endothermic event at 172-176° C. In further embodiments of Salt 6, the present disclosure provides:
6.1 Salt 6, wherein the salt has a 1:1 molar ratio of lumateperone to benzenesulfonic acid (i.e., a mono-besylate salt).
6.2 Salt 6, wherein the salt has a 1:2 molar ratio of lumateperone to benzenesulfonic acid (i.e., a bis-besylate salt).
6.3 Any foregoing form of Salt 6 which is a solvate, e.g., an ethyl acetate solvate, or a toluene solvate.
6.4 Any foregoing form of Salt 6 which is not a solvate.
6.5 Any foregoing form of Salt 6 which is a hydrate.
6.6 Any foregoing form of Salt 6 which is not a hydrate.
6.7 Any foregoing form of Salt 6 formed by combining lumateperone free base and benzenesulfonic acid in a molar ratio from 1:0.5 to 1:3, e.g., a 1:0.75 to 1:1.5 molar ratio, or a 1:0.75 to 1.25 molar ratio, or a 1:1.5 to 1:2.5 molar ratio, or a 1:1.75 to 1:2.25 molar ratio, or a 1:1.75 to 1:3 molar ratio, or a 1:1 to 1:2 molar ratio, or about a 1:1 molar ratio, or about a 1:1.5, or about a 1:2 molar ratio, or about a 1:2.5 molar ratio.
6.8 Any foregoing form of Salt 6, wherein the salt is in a homogeneous crystal form, free or substantially free of other forms (e.g., alternative crystal forms or amorphous forms), e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. %, of other forms (e.g., alternative crystal forms or amorphous forms).
6.9 Any foregoing form of Salt 6 in crystalline form, when crystallized from a mixture of benzenesulfonic acid and lumateperone free base, e.g., in an organic solvent, e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof; e.g., ethyl acetate or toluene, optionally wherein the benzenesulfonic acid and the lumateperone free base are in a molar ratio of about 1:1 or about 1:2.
6.10 Salt 6.9, wherein the salt is crystallized from the solvent after an anti-solvent is added, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane.
6.11 Any of Salts 6 or 6.1-6.10, wherein the salt is formed from a 1:1 or 1:2 molar ratio of lumateperone free base to benzenesulfonic acid in ethyl acetate solvent.
6.12 Salt 6.11, wherein a proton-NMR analysis of the salt shows a molar ratio of lumateperone to benzenesulfonic acid of about 1:1.
6.13 Salt 6.11 or 6.12, wherein a DSC analysis shows one endothermic event at about 96° C. (e.g., a desolvation), and/or one endothermic event at about 110° C. (e.g., a melt).
6.14 Any form of Salt 6.11-6.13, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 2, 6, 7, 10, 11, 12, 13, 17, 19, 20, 24, 25, 26, and/or 28:

XRPD (Cu anode, Ni filter) for Benzenesulfonate
Salt Crystal Polymorph 1

| # | Angle | d Value | Rel. Intensity |
|---|---|---|---|
| 1 | 5.807 | 15.20724 | E+03 70.20% |
| 2 | 5.879 | 15.02115 | 54.50% |
| 3 | 8.810 | 10.02881 | 11.60% |
| 4 | 11.418 | 7.74363 | 15.30% |
| 5 | 11.733 | 7.53663 | 17.30% |
| 6 | 12.021 | 7.35627 | 45.10% |
| 7 | 12.389 | 7.13857 | 38.00% |
| 8 | 13.260 | 6.67196 | 20.40% |
| 9 | 15.777 | 5.61243 | 36.30% |
| 10 | 15.944 | 5.55401 | 80.80% |
| 11 | 16.114 | 5.49583 | 83.80% |
| 12 | 16.652 | 5.31950 | 60.00% |
| 13 | 17.003 | 5.21066 | 83.50% |
| 14 | 17.587 | 5.03871 | 13.90% |
| 15 | 17.812 | 4.97576 | 20.60% |
| 16 | 18.016 | 4.91984 | 8.20% |
| 17 | 18.283 | 4.84860 | 45.00% |
| 18 | 18.658 | 4.75200 | 15.90% |
| 19 | 19.358 | 4.58153 | 58.70% |
| 20 | 19.927 | 4.45216 | 38.30% |
| 21 | 20.196 | 4.39339 | 8.50% |
| 22 | 20.529 | 4.32286 | 19.20% |
| 23 | 21.207 | 4.18609 | 13.40% |
| 24 | 22.598 | 3.93146 | 100.00% |
| 25 | 22.980 | 3.86708 | 59.20% |
| 26 | 23.301 | 3.81451 | 51.30% |
| 27 | 23.718 | 3.74831 | 22.40% |
| 28 | 24.086 | 3.69188 | 61.20% |
| 29 | 24.435 | 3.63990 | 9.20% |
| 30 | 25.522 | 3.48730 | 16.00% |
| 31 | 26.195 | 3.39921 | 15.90% |
| 32 | 26.325 | 3.38274 | 15.90% |
| 33 | 27.246 | 3.27051 | 10.30% |
| 34 | 27.962 | 3.18835 | 12.70% |
| 35 | 30.132 | 2.96347 | 7.50% |

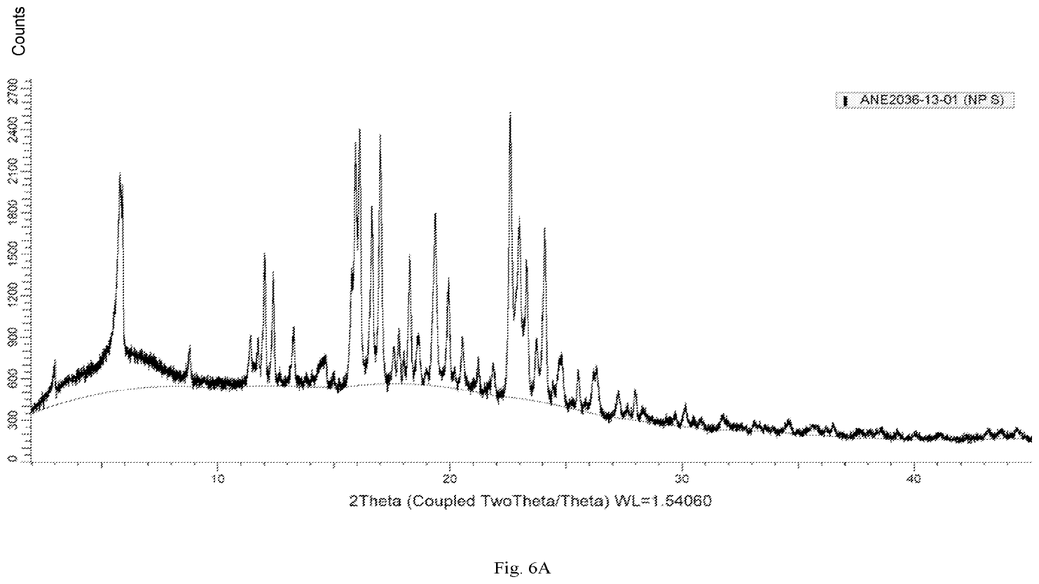

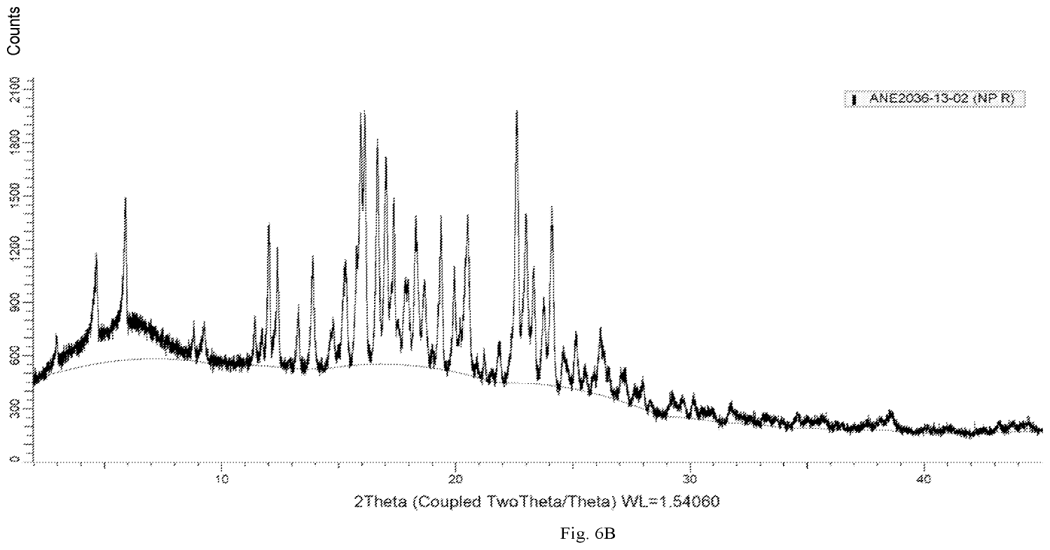

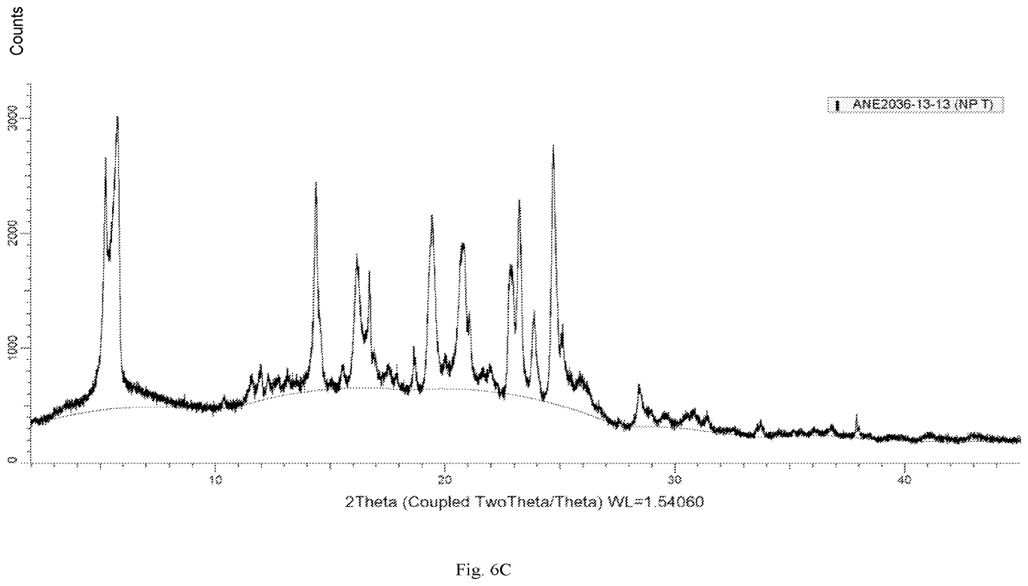

6.15 Any form of Salt 6.11-6.14, in the form of a crystal having an X-ray powder diffraction pattern corresponding to FIG. 6A, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 6A generated using an X-ray diffractometer with a copper anode and a nickel filter.
6.16 Any form of Salt 6.11-6.15, in the form of a crystal having an X-ray powder diffraction pattern having at least 5, or at least 6, or at least 7, or at least 8, peaks having angle (2-theta) values selected from the group consisting of about 5.81, 5.88, 8.81, 11.42, 11.73, 12.02, 12.39, 13.26, 15.78, 15.94, 16.11, 16.65, 17.00, 17.59, 17.81, 18.02, 18.28, 18.66, 19.36, 19.93, 20.20, 20.53, 21.21, 22.60, 22.98, 23.30, 23.72, 24.09, 24.44, 25.52, 26.20, 26.33, 27.25, 27.96, and 30.13, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
6.17 Any form of Salt 6.11-6.16, in the form of a crystal having an X-ray powder diffraction pattern having at least 5, or at least 6, or at least 7, or at least 8, peaks having d-spacing values selected from the group consisting of about 15.21, 15.02, 10.03, 7.74, 7.54, 7.36, 7.14, 6.67, 5.61, 5.55, 5.50, 5.32, 5.21, 5.04, 4.98, 4.92, 4.85, 4.75, 4.58, 4.45, 4.39, 4.32, 4.19, 3.93, 3.87, 3.81, 3.75, 3.69, 3.64, 3.49, 3.40, 3.38, 3.27, 3.19, and 2.96, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
6.18 Any form of Salt 6.11-6.17, in the form of a crystal having an X-ray powder diffraction pattern having at least 5, or at least 6, or at least 7, or at least 8, peaks having angle (2-theta) values and/or d-spacing values as provided in 6.16 and 6.17.
6.19 Any of Salts 6 or 6.1-6.10, wherein the salt is formed from a 1:1 or 1:2 molar ratio of lumateperone free base to benzenesulfonic acid in toluene solvent.
6.20 Salt 6.19, wherein a proton-NMR analysis of the salt shows a molar ratio of lumateperone to benzenesulfonic acid of about 1:1.
6.21 Salt 6.19 or 6.20, wherein a DSC analysis shows one endothermic event at about 131° C. (e.g., a melt).
6.22 Any form of Salt 6.19-6.21, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 1, 2, 4, 5, 7, 10, 11, 12, 13, 14, 17, 19, 21, 25, 25, and/or 28:

XRPD (Cu anode, Ni filter) for Benzenesulfonate
Salt Crystal Polymorph 2

| # | Angle | d Value | Rel. Intensity |
|---|---|---|---|
| 1 | 4.655 | 18.96770 | 37.60% |
| 2 | 5.901 | 14.96484 | 58.00% |
| 3 | 11.434 | 7.73279 | 20.50% |
| 4 | 12.022 | 7.35562 | 52.90% |
| 5 | 12.404 | 7.13035 | 44.40% |
| 6 | 13.269 | 6.66704 | 17.50% |
| 7 | 13.893 | 6.36900 | 39.00% |
| 8 | 14.765 | 5.99485 | 17.40% |
| 9 | 15.290 | 5.79017 | 34.50% |
| 10 | 15.964 | 5.54740 | 87.00% |
| 11 | 16.112 | 5.49647 | 87.10% |
| 12 | 16.673 | 5.31286 | 76.80% |
| 13 | 17.025 | 5.20374 | 74.90% |
| 14 | 17.359 | 5.10456 | 63.20% |
| 15 | 17.848 | 4.96561 | 29.50% |
| 16 | 17.949 | 4.93789 | 31.50% |
| 17 | 18.290 | 4.84677 | 53.30% |
| 18 | 18.649 | 4.75407 | 26.30% |
| 19 | 19.374 | 4.57783 | 54.50% |
| 20 | 19.934 | 4.45050 | 32.50% |
| 21 | 20.518 | 4.32524 | 58.60% |
| 22 | 21.215 | 4.18450 | 11.00% |
| 23 | 21.845 | 4.06526 | 15.40% |
| 24 | 22.603 | 3.93072 | 100.00% |
| 25 | 22.980 | 3.86696 | 66.10% |
| 26 | 23.317 | 3.81191 | 45.50% |
| 27 | 23.753 | 3.74293 | 27.70% |
| 28 | 24.099 | 3.68997 | 69.70% |
| 29 | 25.119 | 3.54231 | 19.50% |
| 30 | 26.174 | 3.40189 | 26.60% |
| 31 | 27.970 | 3.18742 | 12.60% |
| 32 | 30.127 | 2.96396 | 6.90% |

6.23 Any form of Salt 6.19-6.22, in the form of a crystal having an X-ray powder diffraction pattern corresponding to FIG. 6B, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 6B generated using an X-ray diffractometer with a copper anode and a nickel filter.
6.24 Any form of Salt 6.19-6.23, in the form of a crystal having an X-ray powder diffraction pattern having at least 5, or at least 6, or at least 7, or at least 8, peaks having angle (2-theta) values selected from the group consisting of about 4.66, 5.90, 11.43, 12.02, 12.40, 13.27, 13.89, 14.77, 15.29, 15.96, 16.11, 16.67, 17.03, 17.36, 17.85, 17.95, 18.29, 18.65, 19.37, 19.93, 20.52, 21.22, 21.85, 22.60, 22.98, 23.32, 23.75, 24.10, 25.12, 26.17, 27.97, and 30.13, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
6.25 Any form of Salt 6.19-6.24, in the form of a crystal having an X-ray powder diffraction pattern having at least 5, or at least 6, or at least 7, or at least 8, peaks having d-spacing values selected from the group consisting of about 18.97, 14.96, 7.73, 7.36, 7.13, 6.67, 6.37, 5.99, 5.79, 5.55, 5.50, 5.31, 5.20, 5.10, 4.97, 4.94, 4.85, 4.75, 4.58, 4.45, 4.33, 4.18, 4.07, 3.93, 3.87, 3.81, 3.74, 3.69, 3.54, 3.40, 3.19, and 2.96, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
6.26 Any form of Salt 6.19-6.25, in the form of a crystal having an X-ray powder diffraction pattern having at least 5, or at least 6, or at least 7, or at least 8, peaks having angle (2-theta) values and/or d-spacing values as provided in 6.24 and 6.25.
6.27 Any of Salts 6 or 6.1-6.10, wherein the salt is formed from a 1:1 molar ratio of lumateperone free base to benzenesulfonic acid in ethyl acetate solvent.
6.28 Salt 6.27, wherein a proton-NMR analysis of the salt shows a molar ratio of lumateperone to benzenesulfonic acid of about 1:1.
6.29 Salt 6.27 or 6.28, wherein a DSC analysis shows one endothermic event at about 110° C. (e.g., a melt), and/or one endothermic event at about 126° C. (e.g., a melt).
6.30 Any form of Salt 6.27-6.29, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 2, 2, 3, 4, 5, 8, 14, and/or 16:

XRPD (Cu anode, Ni filter) for Benzenesulfonate
Salt Crystal Polymorph 3

| # | Angle | d Value | Rel. Intensity |
|---|---|---|---|
| 1 | 5.240 | 16.85241 | 89.90% |
| 2 | 5.756 | 15.34242 | 100.00% |
| 3 | 14.387 | 6.15169 | 73.40% |
| 4 | 16.165 | 5.47865 | 47.20% |
| 5 | 16.713 | 5.30036 | 45.40% |
| 6 | 18.653 | 4.75319 | 21.10% |
| 7 | 19.249 | 4.60734 | 31.00% |
| 8 | 19.441 | 4.56222 | 62.80% |
| 9 | 20.697 | 4.28806 | 50.40% |
| 10 | 20.839 | 4.25914 | 50.10% |
| 11 | 21.084 | 4.21030 | 28.80% |
| 12 | 22.806 | 3.89608 | 48.00% |
| 13 | 22.907 | 3.87919 | 46.20% |
| 14 | 23.212 | 3.82890 | 71.20% |
| 15 | 23.874 | 3.72418 | 30.60% |
| 16 | 24.695 | 3.60221 | 99.90% |
| 17 | 25.066 | 3.54981 | 30.10% |
| 18 | 25.127 | 3.54131 | 24.10% |
| 19 | 26.712 | 3.33459 | 6.90% |
| 20 | 28.440 | 3.13583 | 14.30% |
| 21 | 33.755 | 2.65326 | 6.00% |
| 22 | 37.907 | 2.37161 | 11.30% |

6.31 Any form of Salt 6.27-6.30, in the form of a crystal having an X-ray powder diffraction pattern corresponding to FIG. 6C, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 6C generated using an X-ray diffractometer with a copper anode and a nickel filter.
6.32 Any form of Salt 6.27-6.31, in the form of a crystal having an X-ray powder diffraction pattern having at least 5, or at least 6, or at least 7, or at least 8, peaks having angle (2-theta) values selected from the group consisting of about 5.24, 5.76, 14.39, 16.17, 16.71, 18.65, 19.25, 19.44, 20.70, 20.84, 21.08, 22.81, 22.91, 23.21, 23.87, 24.70, 25.07, 25.13, 26.71, 28.44, 33.76, and 37.91, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
6.33 Any form of Salt 6.27-6.32, in the form of a crystal having an X-ray powder diffraction pattern having at least 5, or at least 6, or at least 7, or at least 8, peaks having d-spacing values selected from the group consisting of about 16.85, 15.34, 6.15, 5.48, 5.30, 4.75, 4.61, 4.56, 4.29, 4.26, 4.21, 3.90, 3.88, 3.83, 3.72, 3.60, 3.55, 3.54, 3.33, 3.14, 2.65, and 2.37, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
6.34 Any form of Salt 6.27-6.33, in the form of a crystal having an X-ray powder diffraction pattern having at least 5, or at least 6, or at least 7, or at least 8, peaks having angle (2-theta) values and/or d-spacing values as provided in 6.32 and 6.33.
6.35 Any foregoing form of Salt 6, wherein the lumateperone is deuterated, e.g., wherein the deuterium:protium ratio at one or more specified positions in the molecule is significantly higher, e.g., at least 2×, for example at least 10× higher, than the natural isotope ratios or the isotope ratios at other positions in the molecule; for example, any foregoing form of Salt 1 wherein either or both of the —CH₂— moieties of the piperazine ring are deuterated, e.g., —CHD- or —CD₂-, at levels which are significantly higher than the natural deuterium:protium isotope ratio or the deuterium:protium isotope ratio at other positions in the molecule, and/or wherein the methyl group on the piperazine ring is deuterated, e.g., is CD₃-, e.g., at levels which are significantly higher than the natural deuterium:protium isotope ratio or the deuterium:protium isotope ratio at other positions in the molecule; for example, wherein the deuterated lumateperone is any deuterated lumateperone as described in US 2019/0231780 or US 2021/0008065, the contents of each of which are hereby incorporated by reference in their entireties.
6.36 Any foregoing form of Salt 6 exhibiting any combination of characteristics as described in 6.1-6.35.

In a seventh embodiment, the present disclosure provides lumateperone in the form of an (alkylsulfonic acid addition salt (Salt 7), e.g., wherein the alkylsulfonic acid is a (C₃₋₁₂alkyl)sulfonic acid, or a (C₄₋₁₂alkyl)sulfonic acid, or a (C₅₋₁₂alkyl)sulfonic acid, or a (C₃₋₁₀alkyl)sulfonic acid, or a (C₄₋₁₀alkyl)sulfonic acid, or a (C₅₋₁₀alkyl)sulfonic acid, such as a pentane-1-sulfonate salt, or a heptane-1-sulfonate salt. In further embodiments of Salt 7, the present disclosure provides:
7.1 Salt 7 in solid form.
7.2 Salt 7 or 7.1 in crystalline form, e.g., dry crystalline form.
7.3 Salt 7, 7.1, or 7.2, wherein the salt has a 1:1 molar ratio of lumateperone to the alkylsulfonic acid.
7.4 Salt 7, 7.1, or 7.2, wherein the salt has a 1:2 molar ratio of lumateperone to the alkylsulfonic acid.
7.5 Any foregoing form of Salt 4 which is a solvate, e.g., an ethyl acetate solvate, or a toluene solvate.
7.6 Any foregoing form of Salt 7 which is not a solvate.
7.7 Any foregoing form of Salt 7 which is a hydrate.
7.8 Any foregoing form of Salt 7 which is not a hydrate.
7.9 Any foregoing form of Salt 7 formed by combining lumateperone free base and the alkylsulfonic acid in a molar ratio from 1:0.5 to 1:3, e.g., a 1:0.75 to 1:1.5 molar ratio, or a 1:0.75 to 1.25 molar ratio, or a 1:1.5 to 1:2.5 molar ratio, or a 1:1.75 to 1:2.25 molar ratio, or a 1:1.75 to 1:3 molar ratio, or a 1:1 to 1:2 molar ratio, or about a 1:1 molar ratio, or about a 1:1.5, or about a 1:2 molar ratio, or about a 1:2.5 molar ratio.
7.10 Any foregoing form of Salt 7 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. %, of amorphous forms.
7.11 Any foregoing form of Salt 7 in crystalline form, when crystallized from a mixture of the alkylsulfonic acid and lumateperone free base, e.g., in an organic solvent, e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof; e.g., ethyl acetate or toluene, optionally wherein the lumateperone free base and the alkylsulfonic acid are in a molar ratio of about 1:1 or about 1:2.
7.12 Salt 7.11, wherein the salt is crystallized from the solvent after an anti-solvent is added, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane, e.g., wherein the organic solvent is toluene and the anti-solvent is heptane.
7.13 Any of Salts 7 or 7.1-7.12, wherein the alkylsulfonic acid is selected from propane-1-sulfonic acid, butane-1-sulfonic acid, pentane-1-sulfonic acid, hexane-1-sulfonic acid, heptane-1-sulfonic acid, octane-1-sulfonic acid, nonane-1-sulfonic acid, decane-1-sulfonic acid, undecane-1-sulfonic acid, and dodecane-1-sulfonic acid, or any branched isomer thereof (e.g., 4-methylpentane-1-sulfonic acid, 4,4-dimethylpentane-1-sulfonic acid, 3,4-dimethylpentane-1-sulfonic acid, 5-methylhexane-1-sulfonic acid, 3,5-dimethylhexane-1-sulfonic acid, 3-ethylpentane-1-sulfonic acid, 3-ethylhexane-1-sulfonic acid, etc.).
7.14 Salt 7, or any of 7.1-7.13, wherein the salt has an aqueous solubility of less than 20 mg/mL, e.g., less than 15 mg/mL, or less than 10 mg/mL, or less than 5 mg/mL, or less than 3 mg/mL, and/or at least 0.001 mg/mL, or at least 0.01 mg/mL, or at least 0.1 mg/mL or at least 1 mg/mL, e.g., at a pH of 5-8, or at a pH of 6-8, or at a pH of 7-8, or at a pH of 7-7.5.
7.15 Salt 7, or any of 7.1-7.13, wherein the salt has an aqueous solubility of less than 2 mg/mL, or less than 1 mg/mL, or less than 0.5 mg/mL, or less than 0.1 mg/mL, and/or at least 0.001 mg/mL, or at least 0.01 mg/mL, or at least 0.1 mg/mL or at least 1 mg/mL, e.g., at a pH of 5-8, or at a pH of 6-8, or at a pH of 7-8, or at a pH of 7-7.5.
7.16 Any of Salts 7 or 7.1-7.15, wherein the alkylsulfonic acid is pentane-1-sulfonic acid, and the salt is formed from a 1:2 molar ratio of lumateperone free base to pentane-1-sulfonic acid in ethyl acetate solvent.
7.17 Salt 7.16, wherein a proton-NMR analysis of the salt shows a molar ratio of lumateperone to pentane-1-sulfonic acid of about 1:2.
7.18 Salt 7.16 or 7.17, wherein a DSC analysis shows one endothermic event at about 141° C. (e.g., a melt).
7.19 Any form of Salt 7.16-7.18, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 2, 5, 6, 7, 9, and/or 12:

XRPD (Cu anode, Ni filter) for Pentane-1-Sulfonate Salt Crystal

| # | Angle | d Value | Rel. Intensity |
|---|---|---|---|
| 1 | 2.086 | 42.32365 | 2.40% |
| 2 | 3.777 | 23.37371 | 100.00% |
| 3 | 7.487 | 11.79790 | 19.80% |
| 4 | 11.225 | 7.87635 | 5.70% |
| 5 | 14.766 | 5.99468 | 32.90% |
| 6 | 16.224 | 5.45895 | 20.60% |
| 7 | 16.561 | 5.34869 | 28.00% |
| 8 | 17.339 | 5.11045 | 17.80% |
| 9 | 17.757 | 4.99095 | 29.40% |
| 10 | 18.639 | 4.75678 | 14.70% |
| 11 | 19.663 | 4.51123 | 17.30% |
| 12 | 20.014 | 4.43290 | 54.80% |
| 13 | 20.344 | 4.36175 | 28.70% |
| 14 | 20.782 | 4.27088 | 16.80% |
| 15 | 21.353 | 4.15784 | 16.10% |
| 16 | 21.738 | 4.08517 | 20.60% |
| 17 | 22.567 | 3.93679 | 15.00% |
| 18 | 25.287 | 3.51922 | 17.40% |
| 19 | 38.261 | 2.35050 | 3.90% |

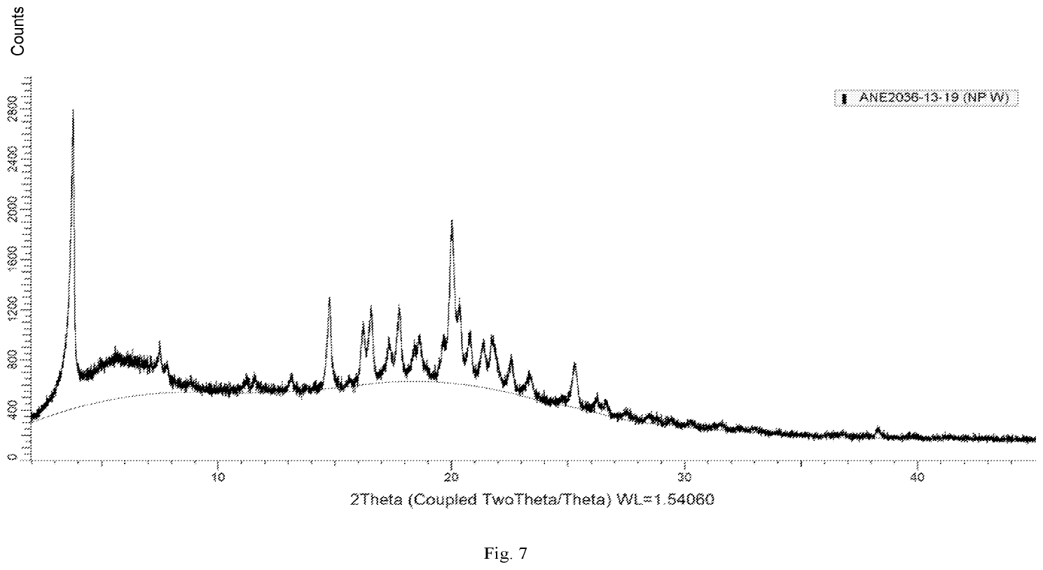

7.20 Any form of Salt 7.16-7.19, in the form of a crystal having an X-ray powder diffraction pattern corresponding to FIG. 7, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 7 generated using an X-ray diffractometer with a copper anode and a nickel filter.
7.21 Any form of Salt 7.16-7.20, in the form of a crystal having an X-ray powder diffraction pattern having at least 5, or at least 6, or at least 7, or at least 8, peaks having angle (2-theta) values selected from the group consisting of about 2.09, 3.78, 7.49, 11.23, 14.77, 16.22, 16.56, 17.34, 17.76, 18.64, 19.66, 20.01, 20.34, 20.78, 21.35, 21.74, 22.57, 25.29, and 38.26, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
7.22 Any form of Salt 7.16-7.21, in the form of a crystal having an X-ray powder diffraction pattern having at least 5, or at least 6, or at least 7, or at least 8, peaks having d-spacing values selected from the group consisting of about 42.32, 23.37, 11.80, 7.88, 5.99, 5.46, 5.35, 5.11, 4.99, 4.76, 4.51, 4.43, 4.36, 4.27, 4.16, 4.09, 3.94, 3.52, and 2.35, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
7.23 Any form of Salt 7.16-7.22, in the form of a crystal having an X-ray powder diffraction pattern having at least 5, or at least 6, or at least 7, or at least 8, peaks having angle (2-theta) values and/or d-spacing values as provided in 7.19 and 7.20.
7.24 Any of Salts 7 or 7.1-7.15, wherein the alkylsulfonic acid is heptane-1-sulfonic acid and the salt is formed from a 1:2 molar ratio of lumateperone free base to heptane-1-sulfonic acid in ethyl acetate solvent.
7.25 Salt 7.24, wherein a proton-NMR analysis of the salt shows a molar ratio of lumateperone to heptane-1-sulfonic acid of about 1:2.
7.26 Salt 7.24 or 7.25, wherein a DSC analysis shows one endothermic event at about 151° C. (e.g., a melt).
7.27 Any form of Salt 7.24-7.26, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength (e.g., any one or more peaks shifted by up to +/−0.2 degrees angle or up to +/−0.2 d-spacing), e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 1, 6, 12, 13, 14, 16, 17, 20, and/or 23:

XRPD (Cu anode, Ni filter) for Heptane-1-Sulfonate Salt Crystal

| # | Angle | d Value | Rel. Intensity |
|---|---|---|---|
| 1 | 3.491 | 25.29231 | 100.00% |
| 2 | 6.836 | 12.92058 | 5.90% |
| 3 | 7.435 | 11.88044 | 5.10% |
| 4 | 10.282 | 8.59661 | 2.50% |
| 5 | 13.686 | 6.46513 | 3.40% |
| 6 | 14.175 | 6.24316 | 11.20% |
| 7 | 14.883 | 5.94771 | 3.60% |
| 8 | 15.750 | 5.62216 | 7.30% |
| 9 | 16.320 | 5.42709 | 6.00% |
| 10 | 16.737 | 5.29276 | 7.80% |
| 11 | 17.121 | 5.17487 | 9.00% |
| 12 | 17.617 | 5.03024 | 14.20% |
| 13 | 18.042 | 4.91282 | 12.60% |
| 14 | 18.345 | 4.83224 | 10.20% |
| 15 | 19.220 | 4.61410 | 5.80% |
| 16 | 19.904 | 4.45723 | 26.70% |
| 17 | 20.184 | 4.39595 | 32.60% |
| 18 | 20.498 | 4.32935 | 7.80% |
| 19 | 21.602 | 4.11051 | 2.30% |
| 20 | 22.401 | 3.96562 | 9.70% |
| 21 | 23.223 | 3.82713 | 6.80% |
| 22 | 24.178 | 3.67808 | 4.80% |
| 23 | 24.844 | 3.58092 | 16.70% |
| 24 | 26.327 | 3.38255 | 2.30% |
| 25 | 27.563 | 3.23360 | 2.70% |
| 26 | 30.590 | 2.92015 | 2.50% |
| 27 | 31.762 | 2.81500 | 1.60% |
| 28 | 32.886 | 2.72129 | 2.20% |
| 29 | 33.600 | 2.66507 | 2.20% |

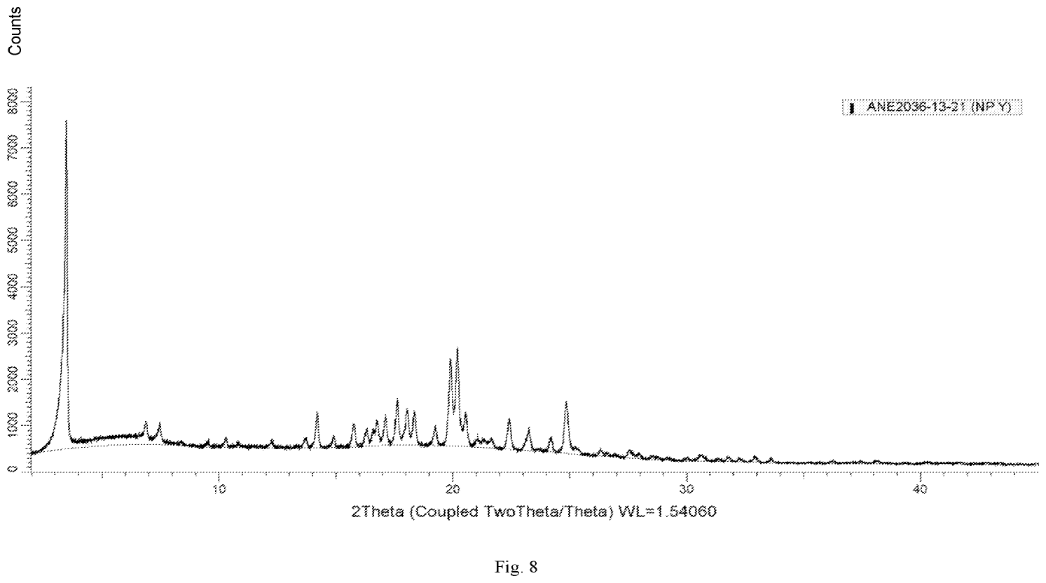

7.28 Any form of Salt 7.24-7.27, in the form of a crystal having an X-ray powder diffraction pattern corresponding to FIG. 8, e.g., taking into account potential variations due to sample purity and instrument variation, for example 2θ shifts due to variation in X-ray wavelength (e.g., any one or more peaks shifted by up to +/−0.2 degrees angle), e.g., an X-ray powder diffraction pattern corresponding to FIG. 8 generated using an X-ray diffractometer with a copper anode and a nickel filter.
7.29 Any form of Salt 7.24-7.28, in the form of a crystal having an X-ray powder diffraction pattern having at least 5, or at least 6, or at least 7, or at least 8, peaks having angle (2-theta) values selected from the group consisting of about 3.49, 6.84, 7.44, 10.28, 13.69, 14.18, 14.88, 15.75, 16.32, 16.74, 17.12, 17.62, 18.04, 18.35, 19.22, 19.90, 20.18, 20.50, 21.60, 22.40, 23.22, 24.18, 24.84, 26.33, 27.56, 30.59, 31.76, 32.89, and 33.60, taking into account potential variations due to sample purity and instrument variation (e.g., any one or more peaks shifted by up to +/−0.2 degrees angle), wherein the X-ray powder diffraction pattern is generated using an X-ray diffractometer with a copper anode and a nickel filter.
7.30 Any form of Salt 7.24-7.29, in the form of a crystal having an X-ray powder diffraction pattern having at least 5, or at least 6, or at least 7, or at least 8, peaks having d-spacing values selected from the group consisting of about 25.29, 12.92, 11.88, 8.60, 6.47, 6.24, 5.95, 5.62, 5.43, 5.29, 5.17, 5.03, 4.91, 4.83, 4.61, 4.46, 4.40, 4.33, 4.11, 3.97, 3.83, 3.68, 3.58, 3.38, 3.23, 2.92, 2.82, 2.72, and 2.67, taking into account potential variations due to sample purity and instrument variation (e.g., any one or more peaks shifted by up to +/−0.2 d-spacing), wherein the X-ray powder diffraction pattern is generated using an X-ray diffractometer with a copper anode and a nickel filter.
7.31 Any form of Salt 7.24-7.30, in the form of a crystal having an X-ray powder diffraction pattern having at least 5, or at least 6, or at least 7, or at least 8, peaks having angle (2-theta) values and/or d-spacing values as provided in 7.27 and 7.28.
7.32 Any foregoing form of Salt 7, wherein the lumateperone is deuterated, e.g., wherein the deuterium:protium ratio at one or more specified positions in the molecule is significantly higher, e.g., at least 2×, for example at least 10× higher, than the natural isotope ratios or the isotope ratios at other positions in the molecule; for example, any foregoing form of Salt 1 wherein either or both of the —CH₂— moieties of the piperazine ring are deuterated, e.g., —CHD- or —CD₂-, at levels which are significantly higher than the natural deuterium:protium isotope ratio or the deuterium:protium isotope ratio at other positions in the molecule, and/or wherein the methyl group on the piperazine ring is deuterated, e.g., is CD₃-, e.g., at levels which are significantly higher than the natural deuterium:protium isotope ratio or the deuterium:protium isotope ratio at other positions in the molecule; for example, wherein the deuterated lumateperone is any deuterated lumateperone as described in US 2019/0231780 or US 2021/0008065, the contents of each of which are hereby incorporated by reference in their entireties.
7.33 Any foregoing form of Salt 7 exhibiting any combination of characteristics as described in 7.1-7.32.

In an eighth embodiment, the present disclosure provides lumateperone in the form of an acid addition salt with a benzenesulfonic acid substituted by one, two, or three groups R, wherein each R is independently a C₁₋₁₂alkyl group (Salt 8), provided that the acid is not p-toluenesulfonic acid, 4-ethylbenzenesulfonic acid, 4-propylbenzenesulfonic acid, 4-t-butylbenzenesulfonic acid, or 4-octylbenzenesulfonic acid. In further embodiments of Salt 8, the present disclosure provides:
8.1 Salt 8, wherein each group R is independently selected from a linear or branched 1-carbon, 2-carbon, 3-carbon, 4-carbon, 5-carbon, 6-carbon, 7-carbon, 8-carbon, 9-carbon, 10-carbon, 11-carbon, or 12-carbon saturated hydrocarbon group.
8.2 Salt 8.1, wherein each group R is independently selected from methyl, ethyl, propyl isopropyl, n-butyl, s-butyl, isobutyl, t-butyl, n-pentyl, s-pentyl, t-pentyl, neopentyl, t-amyl, n-hexyl, n-heptyl, n-octyl and n-decyl.
8.3 Salt 8, wherein the substituted benzenesulfonic acid is selected from meta-toluenesulfonic acid, ortho-toluenesulfonic acid, or a dimethylbenzenesulfonic acid (e.g., 2,4-dimethylbenzenesulfonic acid, 2,5-dimethylbenzenesulfonic acid, 2,6-dimethylbenzenesulfonic acid), or a trimethylbenzenesulfonic acid.
8.4 Salt 8, wherein the acid is a meta-substituted C₂₋₁₂alkylbenzenesulfonic acid, an ortho-substituted C₂₋₁₂alkylbenzenesulfonic acid, a di-(C₂₋₁₂alkyl)benzenesulfonic acid, or a tri-C₂₋₁₂alkylbenzenesulfonic acid, optionally wherein each of said C₂₋₁₂alkyl is selected from ethyl, propyl, isopropyl, n-butyl, sec-butyl, isobutyl, n-pentyl, neopentyl, n-hexyl, n-heptyl, n-octyl, n-nonyl, and n-decyl, or any branched alkyl isomers thereof.
8.5 Any foregoing form of Salt 8 in solid form.
8.6 Any foregoing form of Salt 8 in crystalline form, e.g., dry crystalline form.
8.7 Salt 8, or any of 8.1-8.6, wherein the salt has an aqueous solubility of less than 20 mg/mL, e.g., less than 15 mg/mL, or less than 10 mg/mL, or less than 5 mg/mL, or less than 3 mg/mL, and/or at least 0.001 mg/mL, or at least 0.01 mg/mL, or at least 0.1 mg/mL or at least 1 mg/mL, e.g., at a pH of 5-8, or at a pH of 6-8, or at a pH of 7-8, or at a pH of 7-7.5.
8.8 Salt 8, or any of 8.1-8.6, wherein the salt has an aqueous solubility of less than 2 mg/mL, or less than 1 mg/mL, or less than 0.5 mg/mL, or less than 0.1 mg/mL, and/or at least 0.001 mg/mL, or at least 0.01 mg/mL, or at least 0.1 mg/mL or at least 1 mg/mL, e.g., at a pH of 5-8, or at a pH of 6-8, or at a pH of 7-8, or at a pH of 7-7.5.
8.9 Salt 8, or any of 8.1-8.8, wherein the salt has a 1:1 molar ratio of lumateperone to the substituted benzenesulfonic acid.
8.10 Salt 8, or any of 8.1-8.8, wherein the salt has a 1:2 molar ratio of lumateperone to the substituted benzenesulfonic acid.
8.11 Any foregoing form of Salt 8 which is a solvate, e.g., an ethyl acetate or toluene solvate.
8.12 Any foregoing form of Salt 8 which is not a solvate.
8.13 Any foregoing form of Salt 8 which is a hydrate.
8.14 Any foregoing form of Salt 8 which is not a hydrate.
8.15 Any foregoing form of Salt 8 formed by combining lumateperone free base and the substituted benzenesulfonic acid in a molar ratio from 1:0.5 to 1:3, e.g., a 1:0.75 to 1:1.5 molar ratio, or a 1:0.75 to 1.25 molar ratio, or a 1:1.5 to 1:2.5 molar ratio, or a 1:1.75 to 1:2.25 molar ratio, or a 1:1.75 to 1:3 molar ratio, or a 1:1 to 1:2 molar ratio, or about a 1:1 molar ratio, or about a 1:1.5, or about a 1:2 molar ratio, or about a 1:2.5 molar ratio.
8.16 Any foregoing form of Salt 8 in crystalline form, when crystallized from a mixture of the substituted benzenesulfonic acid and lumateperone free base, e.g., in an organic solvent, e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof; e.g., ethyl acetate or toluene, optionally wherein the lumateperone free base the substituted benzenesulfonic acid are in a molar ratio of about 1:1 or about 1:2.
8.17 Salt 8.16, wherein the salt is crystallized from the solvent after an anti-solvent is added, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane.
8.18 Any foregoing form of Salt 8, wherein the lumateperone is deuterated, e.g., wherein the deuterium:protium ratio at one or more specified positions in the molecule is significantly higher, e.g., at least 2×, for example at least 10× higher, than the natural isotope ratios or the isotope ratios at other positions in the molecule; for example, any foregoing form of Salt 1 wherein either or both of the —CH₂— moieties of the piperazine ring are deuterated, e.g., —CHD- or —CD₂-, at levels which are significantly higher than the natural deuterium:protium isotope ratio or the deuterium:protium isotope ratio at other positions in the molecule, and/or wherein the methyl group on the piperazine ring is deuterated, e.g., is CD₃-, e.g., at levels which are significantly higher than the natural deuterium:protium isotope ratio or the deuterium:protium isotope ratio at other positions in the molecule; for example, wherein the deuterated lumateperone is any deuterated lumateperone as described in US 2019/0231780 or US 2021/0008065, the contents of each of which are hereby incorporated by reference in their entireties

In another embodiment, the present disclosure provides a process (Process 1) for the production of any of salt disclosed herein, comprising the steps of:
(a) reacting lumateperone free base with corresponding acid, together with an organic solvent (e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof), for example, wherein the acid and lumateperone are in a molar ratio of from 1:1 to 1:2, or about 1:1, or about 1:2; optionally at a temperature between 0° C. and 100° C.; and
(b) optionally, subjecting the resulting mixture to a thermocycling protocol (e.g., elevating the temperature to above 50° C., then cooling to 0° C., and optionally repeating this heating and cooling cyclically), or cooling the mixture from its reaction temperature to 5° C. or less; and
(c) optionally, diluting the resulting mixture with an anti-solvent, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane;
   optionally at a temperature between 0° C. and 100° C.; and
(d) optionally performing a second thermocycling protocol (e.g., elevating the temperature to above 50° C., then cooling to 0° C., and optionally repeating this heating and cooling cyclically), or cooling the mixture from its reaction temperature to 5° C. or less; and
(e) recovering the salt thus formed, e.g., recovering a salt according to any of Salt 1, et seq., Salt 2, et seq., Salt 3, et seq., Salt 4, et seq., Salt 5, et seq., Salt 6, et seq., Salt 7, et seq., Salt 8, et seq., or any other salt disclosed herein.

In another embodiment of Process 1, the reaction step (a) comprises dissolving or suspending the lumateperone free base in the organic solvent, e.g., toluene or ethyl acetate, and then adding the acid, or dissolving or suspending the acid in the organic solvent, and then adding the lumateperone free base, or combining the dry acid and the dry lumateperone free base, and then adding the organic solvent. In some embodiments, the organic solvent is a mixture of two solvents, preferably two fully miscible solvents, e.g., water/methanol, water/ethanol, water/isopropanol, ethanol/methanol, ethanol/isopropanol, water/acetonitrile, acetonitrile/methanol, acetonitrile/ethanol, toluene/ethyl acetate, toluene/methyl ethyl ketone, ethyl acetate/methyl ethyl ketone, etc.

In some embodiments of Process 1, a crystalline salt product precipitates upon combining the lumateperone free base, the acid, and the organic solvent(s), at the reaction temperature (e.g., between 0° C. and 100° C., or about 5° C., 10° C., 15° C., 20° C., 25° C., 30° C., 35° C., 40° C., 50° C., 60° C., 70° C., or 80° C.). In some embodiments of Process 1, a crystalline salt product precipitates upon cooling the mixture of the lumateperone free base, the acid, and the organic solvent(s), to a temperature below the reaction temperature (e.g., about 0° C., 5° C., 10° C., or 15° C.).

In some embodiments of Process 1, a crystalline salt product precipitates upon subjecting the mixture of the lumateperone free base, the acid, and the organic solvent(s), to a thermocycling protocol, e.g., elevating the temperature to above 50° C., then cooling to 0° C., and optionally repeating this heating and cooling cyclically). For example, the reaction can be heated to 50° C. or 60° C., held for a period of time (e.g., 15 to 300 minutes, e.g., 60 minutes), then cooled to 0° C. or 5° C., held for a period of time (e.g., 15 to 300 minutes, e.g., 60 minutes), then this heating/cooling may be repeated a second, third, fourth, or fifth time. Optionally, each time the cycle is repeated, the high temperature is reduced by 10° C. For example: 20° C.-50° C.-0° C.-40° C.-0° C.-30° C.-0° C.-20° C.-0° C.-10° C.-0° C.; or 20° C.-50° C.-0° C.-40° C.-0° C.-30° C.-0° C.-20° C.-0° C.

In some embodiments of Process 1, the process step (a) is carried out as a batch process, and in other embodiments the process step (a) is carried out as a continuous (flow) process.

In another embodiment, the present disclosure provides a method of purifying lumateperone, in free or salt form, comprising reacting a crude solution of lumateperone free base with an acid as described herein, to form any salt described herein, and recovering the salt thus formed, e.g., in accordance with Process 1, and optionally converting the salt thus formed back to lumateperone free base or to any other salt form of lumateperone (e.g., a lumateperone monotosylate salt).

In another embodiment, the present disclosure provides a pharmaceutical composition (Composition 1) comprising any salt disclosed herein, e.g., any of Salt 1, et seq., Salt 2, et seq., Salt 3, et seq., Salt 4, et seq., Salt 5, et seq., Salt 6, et seq., Salt 7, et seq., Salt 8, et seq., as active ingredient, in combination or association with a pharmaceutically acceptable diluent or carrier. In some embodiments, this pharmaceutical composition is formulated for oral delivery, e.g., as an enteric tablet or capsule, optionally formulated for sustained or delayed release. In some embodiments, this pharmaceutical composition is formulated for transmucosal delivery, e.g., an oral rapidly dissolving tablet, wafer, or gel, e.g., for administration sublingually, or buccally. In some embodiments, this pharmaceutical composition is formulated for transdermal delivery, e.g., a patch, ointment, or gel, e.g., for administration across the skin into the tissues below the epidermis. In some embodiments, this pharmaceutical composition is formulated for injectable delivery, e.g., as a subcutaneous, intravenous, intraperitoneal, intramuscular, or intrathecal injection, e.g., for immediate release. In some embodiments, this pharmaceutical composition is formulated for sustained or delayed injectable delivery, e.g., as a subcutaneous or intramuscular long-acting injectable (LAI).

Suitable carriers for these pharmaceutical formulations are known in the art, and include that which is disclosed in, e.g., US 2016/0031885, US 2016/0310502, US 2018/271862, US 2021/0315891, US 2020/0220280, and US 2021/0069683, the contents of each of which are hereby incorporated by reference in their entireties.

In some embodiments, the pharmaceutical compositions of the present disclosure comprise the lumateperone salts of the present disclosure as crystalline solids. In some embodiments, the pharmaceutical compositions may comprise the lumateperone salts in the form of amorphous solid dispersions. Amorphous solid dispersions of lumateperone tosylate are disclosed in WO2020/123952 and US 2019/0192511, the contents of each of which are hereby incorporated by reference in their entireties. In an amorphous solid dispersion, the amorphous solid lumateperone salt of the present disclosure is stabilized, e.g., against crystallization, by dispersing it an excipient which stabilizes the amorphous solid, preventing or inhibiting its transition to a crystal. Suitable stabilizing excipients include, but are not limited to, cellulose acetate, cellulose acetate phthalate, methacrylate/methyl acrylate copolymer, hydroxypropyl cellulose, hydroxypropyl methyl cellulose, hydroxypropyl methyl cellulose acetate succinate (HPMC-AS), hydroxypropyl methyl cellulose phthalate (HPMC-P), polyvinyl acetate, polyvinyl pyrrolidone, polyvinyl pyrrolidone/vinyl acetate copolymer, and polyethylene glycol/polyvinyl acetate/polyvinylcaprolactam copolymer. In some embodiments, the salt may be stabilized by dispersing it in a single stabilizing excipient, and in other embodiments, the salt may be stabilized by dispersing it in a combination of two or more stabilizing excipients. In some embodiments, the excipient or excipients may be combined with the lumateperone salt in a weight ratio of lumateperone salt to excipient(s) of 25:75 to 75:25, e.g., 26:74 to 74:26, or 30:70 to 70:30, or 35:65 to 65:35, or 40:60 to 60:40, or 42:58 to 58:42, or 44:56 to 56:44, or 45:55 to 55:45, or 47:53 to 53:47, or 48:52 to 52:48, or 49:51 to 51:49, or about 50:50. In other embodiments, the excipient or excipients may be combined with the lumateperone salt in a weight ratio of lumateperone salt to excipient(s) of 5:95 to 50:50, e.g., 5:95 to 49:51, or 5:95 to 45:55, or 10:90 to 40:60, or 15:85 to 35:65, or 20:80 to 30:70, or 22:78 to 28:82, or 23:77 to 27:83, or 24:76 to 26:74, or about 25:75. In other embodiments, the excipient or excipients may be combined with the lumateperone salt in a weight ratio of lumateperone salt to excipient(s) of 50:50 to 95:5, e.g., 51:49 to 95:5, or 55:45 to 95:5, or 60:40 to 90:10, or 65:45 to 85:15, or 70:30 to 80:20 or about 75:25.

In particular embodiments, the present disclosure provides a long-acting injectable pharmaceutical composition (Composition 2) comprising an acid addition salt of lumateperone having an aqueous solubility of less than 20 mg/mL. In further embodiments of Composition 2, the present disclosure provides:
2.1. Composition 2, wherein the acid addition salt of lumateperone has an aqueous solubility of less than 15 mg/mL, or less than 10 mg/mL, or less than 5 mg/mL, or less than 3 mg/mL, or less than 2 mg/mL, or less than 1 mg/mL, e.g., at a pH of 5-8, or at a pH of 6-8, or at a pH of 7-8, or at a pH of 7-7.5.
2.2. Composition 2, wherein the acid addition salt of lumateperone has an aqueous solubility of less than 0.9 mg/mL, or less than 0.7 mg/mL, or less than 0.5 mg/mL, or less than 0.4 mg/mL, or less than 0.3 mg/mL, or less than 0.2 mg/mL, or less than 0.1 mg/mL, e.g., at a pH of 5-8, or at a pH of 6-8, or at a pH of 7-8, or at a pH of 7-7.5.
2.3. Composition 2, 2.1, or 2.2, wherein the acid addition salt of lumateperone has an aqueous solubility of at least 0.001 mg/mL, or at least 0.01 mg/mL, or at least 0.1 mg/mL or at least 1 mg/mL, e.g., at a pH of 5-8, or at a pH of 6-8, or at a pH of 7-8, or at a pH of 7-7.5.
2.4. Composition 2, or any of 2.1-2.3, wherein the acid addition salt of lumateperone is Salt 1, or any of Salts 1.1-1.22.
2.5. Composition 2, or any of 2.1-2.3, wherein the acid addition salt of lumateperone is Salt 2 or any of 2.1-2.22.
2.6. Composition 2, or any of 2.1-2.3, wherein the acid addition salt of lumateperone is Salt 3 or any of 3.1-3.22.
2.7. Composition 2, or any of 2.1-2.3, wherein the acid addition salt of lumateperone is Salt 4 or any of 4.1-4.38.
2.8. Composition 2, or any of 2.1-2.3, wherein the acid addition salt of lumateperone is Salt 5 or any of 5.1-5.21.
2.9. Composition 2, or any of 2.1-2.3, wherein the acid addition salt of lumateperone is Salt 6 or any of 6.1-6.36.
2.10. Composition 2, or any of 2.1-2.3, wherein the acid addition salt of lumateperone is Salt 7 or any of 7.1-7.33.
2.11. Composition 2, or any of 2.1-2.3, wherein the acid addition salt of lumateperone is Salt 8 or any of 8.1-8.18.
2.12. Composition 2, or any of 2.1-2.11, wherein the acid addition salt of lumateperone is a lumateperone 4-octylbenzenesulfonate salt, e.g., the salt as described in Example 2.
2.13. Composition 2, or any of 2.1-2.11, wherein the acid addition salt of lumateperone is a lumateperone 4-tert-butylbenzenesulfonate salt, e.g., the salt as described in Example 3.
2.14. Composition 2, or any of 2.1-2.11, wherein the acid addition salt of lumateperone is a lumateperone 4-propylbenzenesulfonate salt, e.g., the salt as described in Example 4.
2.15. Composition 2, or any of 2.1-2.11, wherein the acid addition salt of lumateperone is a lumateperone 4-ethylbenzenesulfonate salt, e.g., a salt as described in Example 5.
2.16. Composition 2, or any of 2.1-2.11, wherein the acid addition salt of lumateperone is a lumateperone 2-naphthalenesulfonate salt, e.g., the salt as described in Example 6.
2.17. Composition 2, or any of 2.1-2.11, wherein the acid addition salt of lumateperone is a lumateperone benzenesulfonate salt, e.g., a salt as described in Example 7.
2.18. Composition 2, or any of 2.1-2.11, wherein the acid addition salt of lumateperone is a lumateperone pentane-1-sulfonic acid salt, e.g., the salt as described in Example 8.
2.19. Composition 2, or any of 2.1-2.11, wherein the acid addition salt of lumateperone is a lumateperone hexane-1-sulfonic acid salt, e.g., the salt as described in Example 9.
2.20. Composition 2, or any of 2.1-2.19, wherein the acid addition salt of lumateperone is crystalline.
2.21. Composition 2, or any of 2.1-2.19, wherein the acid addition salt of lumateperone is amorphous, e.g., wherein the composition comprises the salt as an amorphous solid dispersion.
2.22. Composition 2, or any of 2.1-2.21, wherein the composition is formulated for intramuscular injection.
2.23. Composition 2, or any of 2.1-2.21, wherein the composition is formulated for subcutaneous injection.
2.24. Composition 2, or any of 2.1-2.23, wherein the composition further comprises at least one pharmaceutically acceptable, diluent, carrier, or excipient, suitable for injection.
2.25. Composition 2.24, wherein the diluent or carrier comprises water and/or a water-miscible organic solvent.
2.26. Composition 2.24, or 2.25, wherein the diluent, carrier, or excipient comprises a polymer, e.g., a biocompatible and biodegradable polymer, in particular, a polymer which is used to inhibit dissolution of an encapsulated or dispersed drug.
2.27. Composition 2.26, wherein the polymer is selected from a polyester of a hydroxyfatty acid (or derivatives thereof), a polymer of an alkyl alpha-cyanoacrylate (e.g., poly(butyl 2-cyanoacrylate)), a polyalkylene oxalate (e.g., polytrimethylene oxalate or polytetramethylene oxalate), a polyortho ester, a polycarbonate (e.g., polyethylene carbonate or polyethylene-propylene carbonate), a polyortho-carbonate, a polyamino acid (e.g., poly-gamma.-L-alanine, poly-gamma-benzyl-L-glutamic acid, or poly-gamma-methyl-L-glutamic acid), a hyaluronic acid ester, a polylactide (e.g., poly-L-lactide, poly-L-lactic acid, poly-D,L-lactide, poly-D,L-lactic acid), a polyglycolide (e.g., polyglycolide or polyglycolic acid), a polylactide-co-glycolide copolymer (PLGA, e.g., having a lactide to glycolide molar ratio of 75:25 to 50:50 or 50:50 to 90:10, such as PLGA 50:50, PLGA 85:15, or PLGA 90:10), poly(aliphatic carboxylic acids) (e.g., polycitric acid, polymalic acid, poly-beta.-hydroxybutyric acid), copolyoxalates, polycaprolactone, poly(glycolic acid-caprolactone), polydioxanone, poly(acetals), poly(lactic acid-caprolactone), polyanhydrides, 2-hydroxybutyric acid-glycolic acid copolymer, polylactic acid-polyethylene glycol copolymer or polyglycolic acid-polyethylene glycol copolymer, and natural polymers including albumin, casein, and waxes, such as, glycerol mono- and distearate.
2.28. Composition 2.27, wherein the polymer is a polylactide, polyglycolide, or polylactide-co-glycolide copolymer (PLGA), optionally with carboxylic acid end groups or carboxylic ester end groups.
2.29. Composition 2.28, wherein the polylactide-co-glycolide copolymer (PLGA) has a lactide to glycolide molar ratio of 75:25 to 50:50, or 50:50 to 90:10, such as PLGA 50:50, PLGA 85:15, or PLGA 90:10.
2.30. Composition 2.27, 2.28 or 2.29, wherein the PLGA copolymer has a weight-average molecular weight of 5,000 to 500,000 Daltons, or 20,000 to 200,000 Daltons, or 24,000 to 38,000 Daltons, or 113,000 to 159,000 Daltons, e.g., about 113,000 Daltons or about 159,000 Daltons.
2.31. Any of Compositions 2.26-2.30, wherein the acid addition salt of lumateperone is dissolved, dispersed, suspended, or encapsulated, in the polymer, e.g., to form a polymeric matrix, such as in the form of polymeric microspheres (e.g. comprising 1-90 wt. % of the acid addition salt of lumateperone, e.g., 5-50 wt. %, 10-50 wt. %, 20-40 wt. %, 30-50 wt. %, 30-40 wt. %, or 35-40 wt. %, by weight of the microparticles).
2.32. Composition 2.24, or 2.25, wherein the diluent, carrier, or excipient does not comprise any polymer which is used to inhibit dissolution of encapsulated or dispersed drugs, e.g., any polymer recited in embodiment 2.27.
2.33. Composition 2, or any of 2.1-2.23, wherein the composition does not comprise any polymer which is used to inhibit dissolution of encapsulated or dispersed drugs, e.g., any polymer recited in embodiment 2.27.
2.34. Composition 2, or any of 2.1-2.33, wherein the composition does not comprise any non-aqueous organic solvent (e.g., wherein the composition does not comprise dimethylformamide, dimethylacetamide, dimethylsulfoxide, methanol, ethanol, propanol, isopropanol, butanol, acetonitrile, tetrahydrofuran, dioxane, dioxolanes) and/or wherein the composition does comprise any liquid oils (e.g., vegetable oils, mineral oil).
2.35. Composition 2, or any of 2.1-2.34, wherein the composition comprises water (e.g., sterile water for injection) and one or more excipients (e.g., water-soluble excipients), such as, thickening agents, buffering agents, osmotic agents, surfactants, and antioxidants.
2.36. Composition 2.35, wherein the composition comprises one or more thickening agents, e.g., selected from carboxymethyl cellulose, sodium carboxymethyl cellulose, hydroxypropyl cellulose (HPC), hydroxyethyl cellulose (HEC), hydroxypropyl methyl cellulose (HPMC), hydroxypropyl ethyl cellulose (HPEC), other cellulose derivatives, microcrystalline cellulose, non-crystalline cellulose, polyacrylate polymers, polyvinylpyrrolidones, polyvinyl alcohols, and polyethylene glycols (e.g., PEG-400, PEG-600).
2.37. Composition 2.35 or 2.36, wherein the composition comprises one or more surfactants.
2.38. Composition 2.37, wherein the one or more surfactants comprises nonionic surfactants, e.g., selected from sorbitan esters (e.g., sorbitan laurate, sorbitan oleate, sorbitan palmitate, sorbitan stearate), polyoxyethylene sorbitan fatty acid esters (e.g., polysorbate 20, polysorbate 40, polysorbate 60, polysorbate 80), polyoxyethylene alkyl ethers, fatty acid esters (e.g., glycerol monostearate, glycerol monolaurate), polyethoxylated fatty acids or vegetable oils (e.g., polyethoxylated castor oil), poloxamers, and fatty alcohols (e.g., stearyl alcohol, cetyl alcohol, cetostearyl alcohol).
2.39. Composition 2.37, wherein the one or more surfactants comprises cationic surfactants, e.g., quaternary amines, such as cetyl pyridinium chloride, benzalkonium chloride, benzethonium chloride, dimethyldioctadecylammonium chloride.
2.40. Composition 2.37, wherein the one or more surfactants comprises anionic surfactants, e.g., alkyl sulfates such as sodium dodecyl sulfate and sodium lauryl sulfate, alkyl aryl ether polysulfonates, and alkyl sulfosuccinates, such as docusate sodium.
2.41. Any of Compositions 2.35-2.40, wherein the composition comprises one or more bulking agents, e.g., selected from mannitol, sucrose, fructose, maltose, xylitol, glucose, starches, sorbitol, magnesium aluminum silicate, and silica (e.g., colloidal silica).
2.42. Any of Compositions 2.35-2.41, wherein the composition comprises one or more pH-adjusting and/or buffering agents, e.g., selected from hydrochloric acid, citric acid, acetic acid, maleic acid, lactic acid, tartaric acid, sodium hydroxide, sodium carbonate, sodium bicarbonate, potassium hydroxide, potassium carbonate, potassium bicarbonate, sodium phosphates, potassium phosphates, sodium acetate, potassium acetate, sodium citrates, potassium citrates, sodium tartrates, potassium tartrates, sodium maleates, potassium maleates, sodium lactate, potassium lactate, and TRIS (tris(hydroxymethyl)aminomethane).
2.43. Composition 2, or any of 2.1-2.42, wherein the composition comprises one or more antioxidants, e.g., selected from butylated hydroxyanisole, butylated hydroxytoluene, propyl gallate, ascorbic acid, ascorbyl palmitate, sodium ascorbate, potassium ascorbate, sodium iso-ascorbate, tocopherols, dihydroguaretic acid, potassium sorbate, sodium bisulfate, sodium metabisulfate, sodium bisulfite, sodium metabisulfite, sodium EDTA, and sorbic acid.
2.44. Composition 2, or any of Compositions 2.1-2.43, wherein the composition comprises one or more other excipients selected from gelatin, casein, lecithin, dextran, glycerol, propylene glycol, butylene glycol, gum acacia, gum tragacanth, waxes (e.g., animal or plant waxes, including beeswax and carnauba wax), cholesterol, vegetable oils (e.g., coconut oil, soybean oil, peanut oil, sesame oil, cottonseed oil, corn oil, olive oil, castor oil, palm oil, almond oil, and refined fractionated oils thereof), ethyl oleate, isopropyl myristate, benzyl benzoate, sodium chloride, mineral oil, free fatty acids, or synthetic mono- or di-glycerides of fatty acids.
2.45. Composition 2, or any of Compositions 2.1-2.45, wherein the composition does not comprise ethanol, glycerol, propylene glycol, butylene glycol, polyethylene glycol, or liquid vegetable oils as co-solvents.
2.46. Composition 2 or any of 2.1-2.45, wherein the composition comprises the acid addition salt of lumateperone (e.g., lumateperone 4-octylbenzenesulfonate or lumateperone 4-tert-butylbenzenesulfonate), water (e.g., sterile water for injection), a thickening agent (e.g., sodium carboxymethylcellulose), a bulking agent (e.g., mannitol), a non-ionic surfactant (e.g., polysorbate 80), and optionally one or more pH-adjusting or buffering agents (e.g., NaOH or HCl, and/or sodium or potassium phosphates).
2.47. Composition 2 or any of 2.1-2.45, wherein the composition comprises the acid addition salt of lumateperone (e.g., lumateperone 4-octylbenzenesulfonate or lumateperone 4-tert-butylbenzenesulfonate), water (e.g., sterile water for injection), a thickening agent (e.g., sodium carboxymethylcellulose), a bulking agent (e.g., mannitol), and optionally one or more pH-adjusting or buffering agents (e.g., sodium or potassium phosphates, NaOH and/or HCl, and/or sodium or potassium phosphates).
2.48. Composition 2, or any of 2.1-2.47, wherein a unit dose for injection of the composition comprises an amount of the acid addition salt of lumateperone equivalent to 100 to 5000 mg of lumateperone free base, e.g., 100 to 500 mg, or 500 to 1000 mg, or 1000 to 1500 mg, or 1500 to 2000 mg, or 2000 to 3000 mg, or 3000 to 5000 mg, or 100 to 250 mg, or 250 to 500 mg, or 500 to 750 mg, or 750 to 1000 mg, or 1000 to 1250 mg, or 1250 to 1500 mg.
2.49. Composition 2, or any of 2.1-2.48, wherein the composition comprises the acid addition salt of lumateperone at a concentration of 0.1 to 1000 mg/mL (measured by weight of the salt), e.g., 1 to 1000 mg/mL, or 10 to 1000 mg/mL, or 50 to 1000 mg/mL, or 100 to 1000 mg/mL or 250 to 1000 mg/mL, or 500 to 1000 mg/mL, or 750 to 1000 mg/mL, or 1 to 10 mg/mL, or 10 to 50 mg/mL, or 50 to 100 mg/mL, or 100 to 500 mg/mL.
2.50. Composition 2, or any of 2.1-2.49, wherein the unit dose volume for injection of the composition is 0.1 to 5.0 mL, e.g., 0.5 to 5.0 mL, or 1.0 to 5.0 mL, or 2.0 to 5.0 mL, or 3.0 to 5.0 mL, or 4.0 to 5.0 mL, or 0.5 to 4.0 mL, or 1.0 to 4.0 mL, or 2.0 to 4.0 mL, or 3.0 to 4.0 mL, or 0.1 to 3.0 mL, 0.5 to 3.0 mL, or 1.0 to 3.0 mL, or 1.5 to 3.0 mL, or 2.0 to 3.0 mL, or 2.5 to 3.0 mL, or 0.1 to 2.5 mL, 0.5 to 2.5 mL, or 1.0 to 2.5 mL, or 1.5 to 2.5 mL, or 2.0 to 2.5 mL, or 0.1 to 2.0 mL, or 0.5 to 2.0 mL, or 1.0 to 2.0 mL, or 1.5 to 2.0 mL, or 0.1 to 1.5 mL, or 0.5 to 1.5 mL, or 1.0 to 1.5 mL, or 0.1 to 1.0 mL, or 0.5 to 1.0 mL, or 0.1 to 0.5 mL.
2.51. Composition 2, or any of 2.1-2.50, wherein upon injection of the composition, an intramuscular or subcutaneous depot is formed which releases the lumateperone (as a salt or as free base) over a period of up to 180 days, e.g., from 1 week to 6 months, or from 1 month to 6 months, or from 7 days to 14 days, or from 14 days to 30 days, or from 7 days to 30 days, or from 1 month to 2 months, or from 1 month to 3 months, or from 3 months to 6 months.
2.52. Composition 2, or any of 2.1-2.51, wherein the composition has a pH from 4-8, e.g., from 5-8, or from 5-7, or from 6-8, or from 6-7, or from 7-8, or from 6.5-7.5, or from 7-7.5, or from 7.5-8, or from 7.1-7.4, or from 7.2-7.4, or from 7.3-7.4.
2.53. Composition 2, or any of 2.1-2.52, wherein the composition comprises the acid addition salt of lumateperone in an amount of 1-50% by weight of the composition (e.g., 10-40% by weight of the composition).
2.54. Composition 2, or any of 2.1-2.52, wherein the composition comprises one or more diluents, carriers, or excipients, in a net amount of 5-99% by weight of the composition (provided that the combination of components does not exceed 100% by weight of the composition).
2.55. Composition 2, or any of 2.1-2.54, wherein the composition comprise water and/or any water-miscible organic solvents or other liquid diluents or carriers in a net amount of 5-99% by weight of the composition.
2.56. Composition 2, or any of 2.1-2.55, wherein the composition comprise water in an amount of 20-99% by weight of the composition (e.g., 20-90%).
2.57. Composition 2, or any of 2.1-2.56, wherein the composition comprises one or more excipients in a net amount of 1-50% by weight of the composition.
2.58. Composition 2, or any of 2.1-2.56, wherein the composition comprises one or more polymers (e.g., biocompatible and biodegradable polymers, in particular, polymers which are used to inhibit dissolution of an encapsulated or dispersed drug) in a net amount of 0-50% by weight of the composition.
2.59. Composition 2, or any of 2.1-2.58, wherein the composition comprises one or more thickening agents in a net amount of 0-50% by weight of the composition.
2.60. Composition 2, or any of 2.1-2.59, wherein the composition comprises one or more bulking agents in a net amount of 0-50% by weight of the composition.
2.61. Composition 2, or any of 2.1-2.60, wherein the composition comprises one or more buffering agents and/or pH-adjusting agents in a net amount of 0-10% by weight of the composition.
2.62. Composition 2, or any of 2.1-2.61, wherein the composition has the following components:

| | |
|---|---|
| Component | Weight % |

| Lumateperone Salt (e.g., salt of | 1-50% | (e.g., 10-40%) |
|---|---|---|
| Examples 2-9) | | |
| Thickening agent (e.g., sodium | 0-20% | (e.g., 1-20%) |
| carboxymethyl cellulose | | |
| Bulking agent (e.g., mannitol) | 0-20% | (e.g., 1-20%) |
| Buffer (e.g., sodium phosphates) | 0.01-5% | (e.g., 0.1-1%) |
| pH-adjusting agent (e.g., NaOH) | <1% | |
| Water | Q.S. | (e.g., 20-90% |

2.63. Composition 2, or any of 2.1-2.62, wherein the composition is sterile.
2.64. Composition 2, or any of 2.1-2.63, wherein the composition is a homogenous solution.
2.65. Composition 2, or any of 2.1-2.63, wherein the composition is a suspension, e.g., a homogenous suspension, optionally wherein the acid addition salt of lumateperone is the only undissolved component.
2.66. Composition 2, or any of 2.1-2.65, wherein the composition is manufactured as a dried solid comprising the acid addition salt of lumateperone and one or more diluents, carriers, or excipients (e.g., water-soluble excipients), and wherein prior to administration, the solid is reconstituted with sterile water for injection to form the long-acting injectable pharmaceutical composition; optionally wherein the dried solid is free of any liquid solvents or co-solvents (e.g., water, water-miscible solvents, or water-immiscible liquids).
2.67. Composition 2.66, wherein the dried solid is freeze-dried.
2.68. Composition 2.66 or 2.67, wherein the dried solid is in the form of a powder, granules, pellets, or a cake.
2.69. Any of Composition 2.66-2.68, wherein the dried solid is packaged in a vial, sachet, or other single-dose package or container, e.g., wherein sterile water for injection is added into the package or container, or the contents of the package or container are added to sterile water for injection, the combination is mixed, and then the resulting composition is drawn into a syringe and then injected into a patient.
2.70. Any of Composition 2.66-2.68, wherein the dried solid is packaged in a pre-filled syringe (single or double chambered), e.g., wherein sterile water for injection is drawn into the pre-filled syringe, the contents are mixed, and then the resulting composition in the syringe is injected into a patient; optionally wherein the dried solid is packaged in a pre-filled two-compartment syringe wherein one compartment comprises the dried solid and the other compartment comprises the sterile water for injection, and the contexts are mixed by opening or releasing a barrier between the two compartments, followed by mixing of the contents of the compartments, and injection of the resulting composition into the patient.
2.71. Composition 2.70, wherein a homogenous solution is formed after combination of the dried solid and the sterile water for injection.
2.72. Composition 2.70, wherein a suspension is formed after combination of the dried solid and the sterile water for injection, e.g., a uniform or homogenous suspension.
2.73. Composition 2, or any of 2.1-2.72, wherein the composition is intended for administration to a patient in need thereof at a frequency of once per week, once every two weeks, once every three weeks, once per month, once every two months, once every three months, once every four months, once every five months, or once every six months.
2.74. Composition 2, or any of 2.1-2.73, wherein a single dose of the injected composition provides a therapeutically effective plasma level of lumateperone free base and/or a therapeutically effective cerebrospinal fluid (CSF) level of lumateperone free base for a period of 1 week to 6 months, e.g., 1 week to 1 month, or 1 month to 3 months, or 3 months to six months, or about one week, two weeks, three weeks, four weeks, one month, two months, three months, four months, five months, or six months.
2.75. Composition 2, or any of 2.1-2.74, wherein the composition is not an emulsion.

It will be appreciated that long-acting injectable (LAI) compositions have been known for some time, but they are primarily based on two concepts: (1) using a viscous, poorly-water soluble or insoluble polymeric matrix to inhibit the dissolution of a water-soluble active substance, and (2) using a water-insoluble prodrug of a water-soluble active substance. In the case of (1), polymers such as PLGA are used in high amounts to dissolve, disperse, or encapsulate, or to form microspheres encapsulating, the active drug product. Dissolution kinetics are controlled by the degradation of the polymer, such as by the hydrolysis of ester bonds of the polymer. Such LAI compositions can be very difficult to formulate because they are prone to non-linear release kinetics, such as undesired initial burst release of drug. In the case of (2), the prodrug itself is pharmacologically inactive. Thus, the kinetics of release of the active agent are controlled by the rate of prodrug degradation to active drug, such as by the hydrolysis of labile ester or carbamate bonds in the prodrug molecule. Such LAI compositions are also difficult to formulate because the prodrugs often cannot sustain more than a short-term duration of action (i.e. weeks). For example, if the prodrug is too labile, it will provide high initial plasma concentrations, but the depot will be depleted too soon, but if the prodrug is too resistant to hydrolysis, it may be difficult to achieve therapeutic plasma concentrations for a sufficient period of time.

The low-solubility salts of the present disclosure provide a third alternative for LAI formulation. Because of the low aqueous solubility of these salts, the salt itself essentially behaves as a pharmacologically inactive agent. Only a very small amount of the drug dissolves in the body's aqueous compartment, where, as a free base, it can cross the blood-brain barrier to exert its therapeutic effects. As a result, a large loading of drug can be delivered by injection, forming an insoluble tissue depot in the muscular or subcutaneous tissues. “Release” of active drug (lumateperone free base) into the tissues is related only to the rate at which the salt solubilizes. Unlike the prior art LAI(s) of lumateperone, the “release” of the active drug can be made independent of the formulation components (e.g., by avoiding the use of polymers which form a polymeric matrix which inhibits dissolution of the drug substance), and is not dependent on a molecular change to the drug molecule (e.g., a covalent bond breaking). This permits the formulation of a much more efficient and controllable LAI products.

In another aspect, the present disclosure provides any salt disclosed herein, e.g., any of Salt 1, et seq., Salt 2, et seq., Salt 3, et seq., Salt 4, et seq., Salt 5, et seq., Salt 6, et seq., Salt 7, et seq., Salt 8, et seq., for use in treating (or for use in manufacturing a medicament to treat) a disease, disorder, or abnormal condition involving or mediated by the 5-HT_{2A} receptor, serotonin transporter (SERT), and/or dopamine D1/D2 receptor signaling pathways, e.g., a disease, condition, or disorder selected from obesity, anorexia, bulimia, depression, anxiety, psychosis, schizophrenia, migraine, obsessive-compulsive disorder, sexual disorders, attention deficit disorder, attention deficit hyperactivity disorder, sleep disorders, conditions associated with cephalic pain, social phobias, dementia, disorders associated with dementia, post-traumatic stress disorder, impulse control disorder, and intermittent explosive disorder. In preferred embodiments, the present disclosure provides these salts for use in the treatment of the negative symptoms of schizophrenia (or the residual symptoms of schizophrenia), major depressive disorder (MDD), treatment-resistant depression, acute depression, bipolar depression, bipolar I disorder, bipolar II disorder, acute anxiety, schizophrenia comorbid with depression, schizophrenia comorbid with anxiety, and depression or other mood disorders associated with encephalitis or neuroinflammation. Such uses of lumateperone are further described in, for example, US 2011/0071080, US 2015/0072964, US 2015/0080404, US 2016/0310502, US 2021/0060009, US 2021/0000822, and US 2021/0186962, and PCT Application No. PCT/US2023/67204, the contents of each of which are hereby incorporated by reference in their entireties.

In another aspect, the present disclosure provides a method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disease, condition, or disorder selected from obesity, anorexia, bulimia, depression, anxiety, psychosis, schizophrenia, migraine, obsessive-compulsive disorder, sexual disorders, attention deficit disorder, attention deficit hyperactivity disorder, sleep disorders, conditions associated with cephalic pain, social phobias, dementia, disorders associated with dementia, post-traumatic stress disorder, impulse control disorder, and intermittent explosive disorder, comprising administering to a patient in need thereof a therapeutically effective amount of any salt disclosed herein, e.g., any of Salt 1, et seq., Salt 2, et seq., Salt 3, et seq., Salt 4, et seq., Salt 5, et seq., Salt 6, et seq., Salt 7, et seq., Salt 8, et seq.

In another embodiment, the present disclosure provides Composition 1 or any of Composition 2, et seq., for use in treating (or for use in manufacturing a medicament to treat) a disease, disorder, or abnormal condition involving or mediated by the 5-HT_{2A} receptor, serotonin transporter (SERT), and/or dopamine D₁/D₂ receptor signaling pathways, e.g., a disease, condition, or disorder selected from obesity, anorexia, bulimia, depression, anxiety, psychosis, schizophrenia, migraine, obsessive-compulsive disorder, sexual disorders, attention deficit disorder, attention deficit hyperactivity disorder, sleep disorders, conditions associated with cephalic pain, social phobias, dementia, disorders associated with dementia, post-traumatic stress disorder, impulse control disorder, and intermittent explosive disorder. In preferred embodiments, the present disclosure provides these salts for use in the treatment of the negative symptoms of schizophrenia (or the residual symptoms of schizophrenia), major depressive disorder (MDD), treatment-resistant depression, acute depression, bipolar depression, bipolar I disorder, bipolar II disorder, acute anxiety, schizophrenia comorbid with depression, schizophrenia comorbid with anxiety, and depression or other mood disorders associated with encephalitis or neuroinflammation. Such uses of lumateperone are further described in, for example, US 2011/0071080, US 2015/0072964, US 2015/0080404, US 2016/0310502, US 2021/0060009, US 2021/0000822, and US 2021/0186962, and PCT Application No. PCT/US2023/67204, the contents of each of which are hereby incorporated by reference in their entireties.

In another embodiment, the present disclosure provides a method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disease, condition, or disorder selected from obesity, anorexia, bulimia, depression, anxiety, psychosis, schizophrenia, migraine, obsessive-compulsive disorder, sexual disorders, attention deficit disorder, attention deficit hyperactivity disorder, sleep disorders, conditions associated with cephalic pain, social phobias, dementia, disorders associated with dementia, post-traumatic stress disorder, impulse control disorder, and intermittent explosive disorder, comprising administering to a patient in need thereof a therapeutically effective amount of Composition 1 or any of Composition 2, et seq.

In preferred embodiments, the methods and uses described above are for the treatment of a disease, condition, or disorder, selected from the negative symptoms of schizophrenia (or the residual symptoms of schizophrenia), major depressive disorder (MDD), treatment-resistant depression, acute depression, bipolar depression, bipolar I disorder, bipolar II disorder, acute anxiety, schizophrenia comorbid with depression, schizophrenia comorbid with anxiety, and depression or other mood disorders associated with encephalitis or neuroinflammation. Such methods of use for lumateperone are further described in, for example, US 2011/0071080, US 2015/0072964, US 2015/0080404, US 2016/0310502, US 2021/0060009, US 2021/0000822, and US 2021/0186962, and PCT Application No. PCT/US2023/67204, the contents of each of which are hereby incorporated by reference in their entireties.

Where the method of use or treatment pertains to the use of a long-acting injectable composition according to Composition 2 et seq., the method or use may comprise administering to the patient in need thereof the composition, by intramuscular or subcutaneous injection, at a frequency of once per week, once every two weeks, once every three weeks, once per month, once every two months, once every three months, once every four months, once every five months, or once every six months. Preferably, the method provides a therapeutically effective plasma level of lumateperone free base and/or a therapeutically effective cerebrospinal fluid (CSF) level of lumateperone free base for a period of 1 week to 6 months, e.g., 1 week to 1 month, or 1 month to 3 months, or 3 months to six months, or about one week, two weeks, three weeks, four weeks, one month, two months, three months, four months, five months, or six months.

In some embodiments, the method comprises administering a unit dose for injection of the composition comprised an amount of the acid addition salt of lumateperone equivalent to 100 to 5000 mg of lumateperone free base, e.g., 100 to 500 mg, or 500 to 1000 mg, or 1000 to 1500 mg, or 1500 to 2000 mg, or 2000 to 3000 mg, or 3000 to 5000 mg, or 100 to 250 mg, or 250 to 500 mg, or 500 to 750 mg, or 750 to 1000 mg, or 1000 to 1250 mg, or 1250 to 1500 mg.

In some embodiments the dosage is administered as a unit dose volume for injection of the composition is 0.1 to 5.0 mL, e.g., 0.5 to 5.0 mL, or 1.0 to 5.0 mL, or 2.0 to 5.0 mL, or 3.0 to 5.0 mL, or 4.0 to 5.0 mL, or 0.5 to 4.0 mL, or 1.0 to 4.0 mL, or 2.0 to 4.0 mL, or 3.0 to 4.0 mL, or 0.1 to 3.0 mL, 0.5 to 3.0 mL, or 1.0 to 3.0 mL, or 1.5 to 3.0 mL, or 2.0 to 3.0 mL, or 2.5 to 3.0 mL, or 0.1 to 2.5 mL, 0.5 to 2.5 mL, or 1.0 to 2.5 mL, or 1.5 to 2.5 mL, or 2.0 to 2.5 mL, or 0.1 to 2.0 mL, or 0.5 to 2.0 mL, or 1.0 to 2.0 mL, or 1.5 to 2.0 mL, or 0.1 to 1.5 mL, or 0.5 to 1.5 mL, or 1.0 to 1.5 mL, or 0.1 to 1.0 mL, or 0.5 to 1.0 mL, or 0.1 to 0.5 mL.

In some embodiments, the method comprises administering a composition having the acid addition salt of lumateperone at a concentration of 0.1 to 1000 mg/mL (measured by weight of the salt), e.g., 1 to 1000 mg/mL, or 10 to 1000 mg/mL, or 50 to 1000 mg/mL, or 100 to 1000 mg/mL or 250 to 1000 mg/mL, or 500 to 1000 mg/mL, or 750 to 1000 mg/mL, or 1 to 10 mg/mL, or 10 to 50 mg/mL, or 50 to 100 mg/mL, or 100 to 500 mg/mL.

It will be appreciated that the dosage administered, the volume administered, and the concentration administered, will depend on the intended duration of action of the LAI composition. Thus, higher dosages and volumes are required for longer-acting compositions, and smaller doses and volumes, may be sufficient for shorter-acting compositions. Preferably, the compositions are administered as aqueous suspensions. In some embodiments, the compositions are administered by reconstituting a dried solid comprising the acid addition salt of lumateperone and one or more diluents, carriers, or excipients (e.g., water-soluble excipients), with sterile water for injection immediately prior to administration (injection) of the resulting long-acting injectable pharmaceutical composition. The dried solid may be provided as a powder, granules, pellets, or a cake, optionally packaged in a vial, sachet, or pre-filled syringe (single or double chambered). Thus, for a single-chambered syringe, sterile water for injection is drawn into the pre-filled syringe comprising the solid, the contents are mixed, and then the contents of the syringe are injected into a patient. In some embodiments, the dried solid is packaged in a pre-filled two-compartment syringe wherein one compartment comprises the dried solid and the other compartment comprises sterile water for injection. Thus, the contents of the two chambers are mixed immediately prior to use by opening or releasing a barrier between the two compartments, mixing the components of the two compartments, and then injecting the resultant long-acting injectable pharmaceutical composition into the patient. Where the dried solid is packaged in a vial, sachet, or other single-dose package or container, sterile water for injection may be added into the package or container, or the contents of the package or container may be added to sterile water for injection. Then the combination is mixed, and then the resulting long-acting injectable composition is drawn into a syringe and then injected into the patient.

Methods of treatment employing long-acting injectable compositions are particularly beneficial for patients who have poor compliance with taking oral medications, such as patients suffering from schizophrenia, bipolar depression, dementia, and other psychiatric disorders.

Methods for synthesizing lumateperone free base are known, and can be found in, for example, US 2004/0220178, US 2006/148808, US 2010/0113781, and US 2020/0102309, the contents of each of which are hereby incorporated by reference in their entireties.

Although specific embodiments of the present disclosure will now be described with reference to the preparations, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### EXAMPLES

The following equipment and methods are used to isolate and characterize the exemplified salt forms.

X-ray powder diffraction (XRPD): The X-ray powder diffraction studies are performed using a Bruker AXS D2 PHASER in Bragg-Brentano configuration, equipment #1549. The X-ray source is a Cu anode at 30 kV, 10 mA. From beam to source, the slits that are used are a primary axial Soller slit 2.5°, a fixed divergence slit 1.0 mm (=0.61°), an 8.0 mm detector slit and a secondary axial Soller slit 2.5°. For monochromatisation, a Kβ-filter (0.5% Ni) is used. The detector is a linear detector LYNXEYE with receiving slit 5° detector opening. The sample stage is standard rotating (5/min) with beam stop. All measuring conditions are logged in the instrument control file. The software used for data collection is Diffrac. Measurement Centre v4.6. Data analysis is performed using Diffrac.Eva V4.1.1 evaluation software. No background correction or smoothing is applied to the patterns.

Bruker AXS D8 Discovery High-Throughput X-Ray Screening: The X-ray powder diffraction studies are performed using a Bruker AXS D8 discover HTS, equipment #3198. Using a Cu anode at 40 kV, 40 mA; Göbel mirror, line optics. Detector: Linear detector LYNXEYE XE with receiving slit 2.950 detector opening. Measurement conditions: scan range 2-45° 2°, 1 s/step, 0.005°/step, and all measuring conditions are logged in the instrument control file. As system suitability, Corundum powder is measured. The software used for data collection is Diffrac.Commander v7.3.0.0. Data analysis is done using Diffrac.Eva v4.2.1. No background correction or smoothing is applied to the patternsS (NIST standard) is checked daily for peak position, peak shape, intensity, and linearity.

Simultaneous thermogravimetry (TGA) and differential scanning calorimetry (DSC) or TGA/DSC analysis: The TGA/DSC studies were performed using a Mettler Toledo TGA/DSC-3+STARe System with a 34-position auto sampler, equipment #3119/#3287. The samples are made using Al crucibles (40 μl; pierced). Typically, 5-10 mg of sample is loaded into a pre-weighed Al crucible and is kept at 20° C. for 5 minutes, after which it is heated at 10° C./min from 20° C. to 350° C. A nitrogen purge of 40 ml/min is maintained over the sample. The software used for data collection and evaluation is STARe Software v15.00 build 8668. No corrections are applied to the thermogram. As system suitability check indium and zinc are measured. For calibration of the instrument benzophenone, indium, lead, tin, and zinc are used as references.

Differential scanning calorimetry (DSC): The DSC studies were performed using a Mettler Toledo DSC1/DSC3+STARe System, equipment #1564/#3168. The samples are made using Al crucibles (40 μl; pierced). Typically, 1-8 mg of sample is loaded onto a pre-weighed Al crucible and is kept at 20° C. for 5 minutes, after which it is heated at 10° C./min from 20° C. to 350° C. and kept at 350° C. for 1 minute. A nitrogen purge of 40 ml/min is maintained over the sample. The software used for data collection and evaluation is STARe Software v15.00 build 8668. No corrections are applied to the thermogram. As system suitability check indium and zinc are measured. For calibration indium, lead and zinc are used as references.

Polarized light microscopy (PLM): The microscopy studies are performed using an AxioVert 35M, equipped with an AxioCamERc 5s, equipment #1612. The microscope is equipped with four lenses: Zeiss A-Plan 5×/0.12, Zeiss A-Plan 10×/0.25, LD A-Plan 20×/0.30 and Achros TIGMAT 32×/0.40. Data collection and evaluation is performed using Carl Zeiss Zen AxioVision Blue Edition Lite 2012 v1.0.0.0 software. A small amount of sample is loaded on an object glass and carefully spread until a thin layer is obtained.

Proton Nuclear Magnetic Resonance Spectroscopy (¹H-NMR): The NMR studies were performed using a Varian Unity Inova 400 NMR spectrometer, equipment #1857. The spectrometer is equipped with a 5 mm ID probe.

Technobis Crystalline: The experiments are performed using the Technobis Crystalline (equipment #2537). A total of eight small reactors are available, in which 8 mL glass vials are used. For each reactor, the vial is filled with the desired chemicals and closed with the desired cap. A CCD camera is connected to reactors E to H in order to monitor the reaction in time. Data is collected with Crystalline version 2.17.2 and evaluated with CrystalClear version 1.0.1.614.

MYA4: The experiments are performed using the Radleys MYA 4 Reaction station with Process Package (equipment #3181). A total of four reactor stations are available, each can be used on 50 mL-100 mL-250 mL and 500 mL scale. All reactors are equipped with stirrers, thermometers and KNF Simdos 02/10 liquid dosing pumps. The MYA 4 Reaction station is controlled with MYA control software V1.1.1 with custom driver for the KNF Simdos 02-10 liquid dosing pumps.

### Example 1: Salt Crystal Screen

A salt screen is conducted in order to identify new solid, stable, crystalline salts of lumateperone, and in particular with the goal of identifying low solubility salts. Lumateperone free base is a sticky, oily substance with very low water-solubility. Numerous past studies have shown that it is very difficult for form solid, crystalline salts of lumateperone. See, e.g., US 2011/112105, US 2019/0112309, US 2020/247805, US 2020/0157100. However, stable crystalline monotosylate, bistosylate, besylate, napsylate, and napadisylate salts of lumateperone have been described, suggesting that sulfonic acids may be preferable acid counterparts for salt formation. The present study relies on a large set of mostly mono- and di-sulfonic acid salts with bulky and/or apolar side chains.

In the initial screen, thirty-six different sulfonic acids are tested at 1:1 and/or 1:2 molar ratios of free base to acid, using the four solvent systems acetonitrile/water (9:1 v/v), methanol, ethyl acetate, and toluene. These solvents are selected based on the known solubility of lumateperone free base and that of the selected acids. This initial screen is conducted using thermocycling on a Technobis Crystal16 equipment.

Stock solutions of lumateperone free base are prepared at a 25 mg/0.5 mL concentration in each of the 4 selected solvents. The acids are weighed into empty vials to provide 1:1 or 1:2 molar ratios, followed by addition of the lumateperone free base solution. The samples are then subjected to the following thermocycling protocol: 20° C.-60° C.-0° C.-50° C.-0° C.-30° C.-0° C.-20° C.-0° C. The applied heating rate is 20° C./minute, and the applied cooling rate is 0.5° C./min. At the conclusion, each vial is checked for solid material.

The vast majority of the vials have either clear solutions or oils. All of the vials with clear solutions are then subjected to an anti-solvent protocol. If the solvent is acetonitrile/water or methanol, then 0.5 mL of water is added to the vial. If the solvent is ethyl acetate or toluene, then 0.5 mL of heptane is added to the vial. The thermocycling protocol is then repeated.

Vials with solid material (either after initial screen or after anti-solvent treatment) are then centrifuged, the supernatant is removed by pipet, and the solid is transferred to a well plate for XRPD analysis. The resulting XRPD spectrum is evaluated to determine whether it is amorphous, or whether it corresponds to a new crystal diffraction pattern.

A total of 272 counterion/solvent/ratio combinations are tested in this initial screen, and only 35 result in solids, however, seven of the solids are obtained in insufficient amount for analysis (indicating a very low yield of salt, if any salt has formed). Of the 28 solids which are formed in sufficient amount for analysis, XRPD shows that five are amorphous, and 23 are crystalline. Analysis of the crystalline solids by XRPD shows the formation of 19 distinct new XRPD patterns, resulting from 14 acids (out of 36 acids tested).

In a second set of preliminary experiments, each of these 14 acids which resulted in any crystalline solid in the initial screen are tested using an alternative protocol. The specified acid is added to a vial to provide a 1:1 molar ratio of acid to lumateperone free base (25 mg), and then the lumateperone free base solution is added (5 mg/0.5 mL). The solvents selected are based on the results of the initial screen, with 11 experiments conducted using ethyl acetate, 5 using toluene, and 1 each using acetonitrile/water or methanol. The reactions are heated to 50° C. for 2 hours, then are cooled to 5° C. All of the experiments result in a clear solution or oil. Anti-solvent addition was conducted with the clear solutions, as described above. Of the 18 experiments, only two result in solids, both of which are the same 20^{th} distinct new XPRD pattern.

Thus, from a total of 290 experiments, 20 candidate new crystalline lumateperone polymorphs are obtained. The candidate polymorphs are the result of using 14 of the 36 different acids. These 20 candidate salts are then analyzed by DSC and TGA, and it is found that nine are solvated and 10 are non-solvated and anhydrous.

Scale-up and further characterization at a 100 mg scale is then attempted on each of these 20 candidate salt polymorphs using the conditions (molar ratio, solvent) which resulted in the original candidate salt. Only 12 are successfully reproduced. The procedure is as follows: the acid is weighed into an empty vial to provide a 1:1 or 1:2 molar ratio and 2.0 mL of the appropriate lumateperone free base stock solution is added to the vial. The mixture is stirred until a clear solution is obtained. Then, the vial is submitted to the thermocycling protocol: 20° C.-60° C.-0° C.-50° C.-0° C.-30° C.-0° C.-20° C.-0° C. The applied heating rate is 10° C./minute, and the applied cooling rate is 0.5° C./min. At the conclusion, each vial is checked for solid material. If a solid is present, the supernatant liquid is removed by pipet, the solid is dried under vacuum, and the solid is then analyzed by XRPD, DSC-TGA, and proton NMR.

Of the 19 experiments, 12 resulted in a solid showing the same XRPD spectrum as found during the initial screen, and these were thus considered successful scale-ups. Five of the experiments resulted in no formation of a solid. Three experiments resulted in the formation of a solid which, based on DSC-TGA and proton NMR, was found not to be a lumateperone salt. Two experiments resulted in a new crystalline salt form positively identified as a lumateperone salt, but whose XRPD spectrum did not match the spectrum obtained during the initial screen under the same conditions.

The same 100 mg scale up was also attempted using the conditions that resulted in five different amorphous solids during the initial screen, in the hope that on a larger scale these might result in crystallization. Of these five attempts, one resulted in a new crystalline XRPD pattern identified as a lumateperone salt.

These 100 mg scale-up experiments resulted in the formation of three benzenesulfonate crystal polymorphs, one 2-naphthalenesulfonate crystal polymorph, three 4-ethylbenzenesulfonate crystal polymorphs, one 4-propylbenzenesulfonate crystal polymorph, one 4-tert-butylbenzenesulfonate crystal polymorph, one 4-octylbenzenesulfonate crystal polymorph, one pentane-1-sulfonate crystal polymorph, and one heptane-1-sulfonate crystal polymorph, for a total of 12 distinct crystalline polymorphs derived from eight different acids.

Importantly, the acids used in these experiments included several sulfonic acids which did not form any solids, or did not form reproducible crystalline solids, yet these acids have structural similarities to the eight acids which successfully formed reproducible lumateperone salts. For example, the aromatic acids naphthalene-1,5-disulfonic acid and 5-isoquinolinesulfonic acid yielded initial crystalline solids that were not reproducible at scale. In contrast, no significant solids were obtained under any conditions using quinoline-8-sulfonic acid, pyridine-2-sulfonic acid, or pyridine-3-sulfonic acid. Even the acids that did form salts in this study, such as benzenesulfonic acid and 4-ethylbenzenesulfonic acid, did so under only some conditions-certain solvents, but not other solvents, or using a 1:1 molar ratio but not a 1:2 ratio, or vice versa. This underscores the ongoing conclusion based on this study, and those that have come before it, that lumateperone forms solid, crystalline salts under unpredictable circumstances and conditions with unpredictable acids. Yet, it also shows that the mere fact that certain acids did not form a reproducible crystalline salt under the conditions tested here, does not preclude the possibility that such acids can form stable, reproducible, crystalline lumateperone salts given further investigation of alternative conditions. Thus, this is an ongoing area of research.

### Example 2: 4-Octylbenzenesulfonic Acid Salt

Initial screening results demonstrated the formation of a crystalline lumateperone 4-octylbenzenesulfonate salt using a 1:1 molar ratio in ethyl acetate or toluene solvent, and using a 1:2 molar ratio in ethyl acetate or toluene solvent. The same XRPD pattern was obtained in all four all four conditions. No solids were obtained using acetonitrile/water or methanol as solvent with either molar ratio. Initial DSC suggested that this was a non-solvated, anhydrous crystal under each of the four conditions. DSC showed a melting event at 164.6° C. with no weight loss on TGA.

As the same XPRD pattern was obtained using both solvents, the 100 mg scale-up was performed using ethyl acetate at 1:1 and 1:2 molar ratios, and a solid was obtained having the same XRPD pattern as in the 25 mg initial screen. DSC on the solid obtained from the 1:1 molar ratio showed a single melting event at 135.0° C., while DSC on the solid obtained from the 1:2 molar ratio showed a single melting event at 157.6° C. Both are indicative of a non-solvated, anhydrous crystal.

The scale-up was repeated again using a 1:1 molar ratio in ethyl acetate at a 500 mg scale, and again a solid resulted having the same XRPD pattern as previously shown. The XRPD pattern is shown in FIG. 1, and the peak listing is shown in the table below:

XRPD (Cu anode, Ni filter) for 4-
octylbenzenesulfonate Salt Crystal

| # | Angle | d Value | Rel. Intensity |
|---|---|---|---|
| 1 | 9.086 | 9.72473 | 18.60% |
| 2 | 9.192 | 9.6129 | 6.40% |
| 3 | 11.369 | 7.77699 | 13.00% |
| 4 | 11.814 | 7.48515 | 47.70% |
| 5 | 12.671 | 6.98031 | 30.80% |
| 6 | 13.236 | 6.68362 | 8.30% |
| 7 | 14.496 | 6.1056 | 6.30% |
| 8 | 15.289 | 5.79046 | 8.90% |
| 9 | 16.063 | 5.51314 | 86.70% |
| 10 | 16.298 | 5.43443 | 74.80% |
| 11 | 16.867 | 5.25211 | 92.20% |
| 12 | 18.217 | 4.86602 | 33.50% |
| 13 | 18.783 | 4.72062 | 20.00% |
| 14 | 19.425 | 4.56604 | 16.80% |
| 15 | 20.114 | 4.41113 | 14.70% |
| 16 | 20.466 | 4.33592 | 22.50% |
| 17 | 22.615 | 3.92858 | 100.00% |
| 18 | 22.671 | 3.91903 | 98.90% |
| 19 | 23.085 | 3.8496 | 42.20% |
| 20 | 23.663 | 3.75691 | 12.10% |
| 21 | 24.099 | 3.68996 | 17.40% |
| 22 | 24.456 | 3.63683 | 17.00% |
| 23 | 25.965 | 3.42881 | 2.50% |
| 24 | 29.822 | 2.99358 | 3.50% |

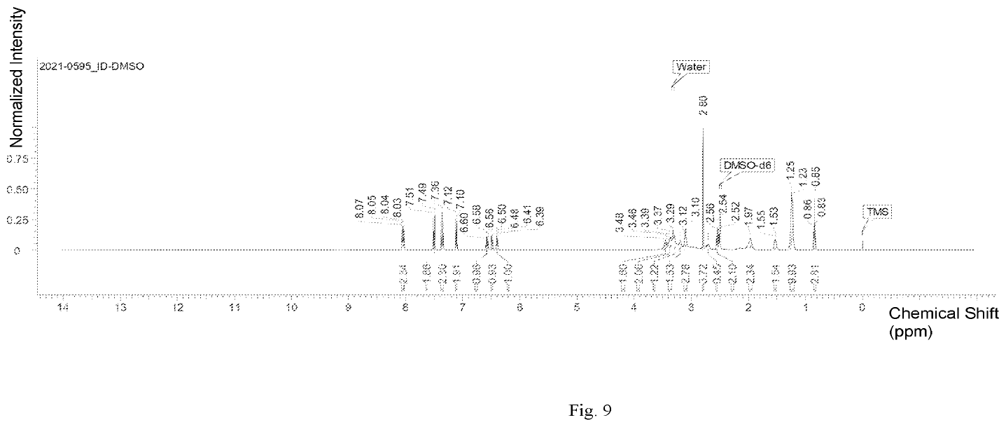

DSC on this solid shows a single melting event at 156.7° C. Proton NMR is conducted and it shows an estimated molar ratio of 1.0:0.8 lumateperone to acid, thus confirming that this is a 1:1 salt. The NMR spectrum is shown in FIG. 9.

### Example 3: 4-Tert-Butylbenzenesulfonic Acid Salt

Initial screening results demonstrated the formation of a crystalline lumateperone 4-tert-butylbenzenesulfonate salt using a 1:2 molar ratio in ethyl acetate solvent. No solids were obtained using acetonitrile/water, methanol, or toluene as solvent, and this acid was not tested at 1:1 molar ratio. Initial DSC suggested that this was a non-solvated, anhydrous crystal. DSC showed a melting event at 210.9° C., and 1.0% weight loss on TGA (suggestive of residual solvent).

The 100 mg scale-up was performed using ethyl acetate at a 1:2 molar ratio, and a solid was obtained having the same XRPD pattern as in the 25 mg initial screen. DSC on the solid showed a single melting event at 211.7° C., as well as a desolvation event at 67.7° C. TGA mass loss was 1.3%, matching the desolvation event. This is indicative of a solvated crystal.

The XRPD pattern is shown in FIG. 2, and the peak listing is shown in the table below:

XRPD (Cu anode, Ni filter) for 4-tert-
butylbenzenesulfonate Salt Crystal

| # | Angle | d Value | Rel. Intensity |
|---|---|---|---|
| 1 | 3.216 | 27.44733 | 13.00% |
| 2 | 3.640 | 24.25154 | 100.00% |
| 3 | 6.665 | 13.25211 | 12.90% |
| 4 | 7.194 | 12.27861 | 20.60% |
| 5 | 13.904 | 6.36395 | 14.90% |
| 6 | 14.724 | 6.01133 | 12.70% |
| 7 | 15.224 | 5.81524 | 14.30% |
| 8 | 15.642 | 5.66062 | 10.10% |
| 9 | 16.050 | 5.51782 | 12.60% |
| 10 | 17.955 | 4.93645 | 8.10% |
| 11 | 18.775 | 4.72263 | 41.10% |
| 12 | 18.903 | 4.69091 | 24.20% |
| 13 | 19.586 | 4.52885 | 17.00% |
| 14 | 20.657 | 4.29632 | 13.60% |
| 15 | 22.084 | 4.02185 | 7.30% |
| 16 | 22.994 | 3.86463 | 5.20% |
| 17 | 23.678 | 3.75456 | 10.20% |

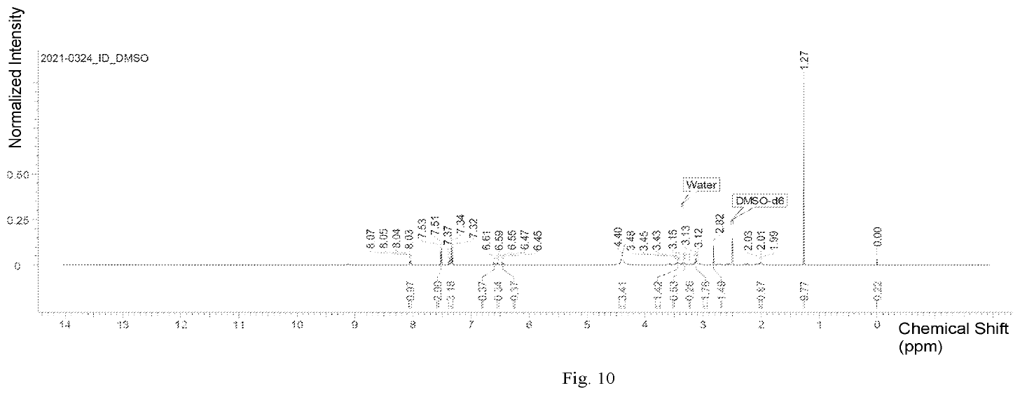

Proton NMR is conducted and it shows an estimated molar ratio of 1:2 lumateperone to acid, thus confirming that this is a 1:2 salt. The NMR spectrum is shown in FIG. 10.

### Example 4: 4-Propylbenzenesulfonic Acid Salt

Initial screening results demonstrated the formation of a crystalline lumateperone 4-propylbenzenesulfonate salt using a 1:2 molar ratio in ethyl acetate solvent. No solids were obtained using acetonitrile/water, methanol, or toluene as solvent, and this acid was not tested at 1:1 molar ratio. Initial DSC suggested that this was a non-solvated, anhydrous crystal. DSC showed a melting event at 158.3° C., and no weight loss on TGA.

The 100 mg scale-up was performed using ethyl acetate at a 1:2 molar ratio, and a solid was obtained having the same XRPD pattern as in the 25 mg initial screen. DSC on the solid showed a single melting event at 160.2° C. TGA mass loss was 0.7%, consistent with residual solvent. This is indicative of a non-solvated, anhydrous crystal.

The XRPD pattern is shown in FIG. 3, and the peak listing is shown in the table below:

XRPD (Cu anode, Ni filter) for 4-propylbenzenesulfonate
Salt Crystal

| # | Angle | d Value | Rel. Intensity |
|---|---|---|---|
| 1 | 4.049 | 21.80405 | 100.00% |
| 2 | 8.007 | 11.03315 | 7.60% |
| 3 | 10.546 | 8.38185 | 6.20% |
| 4 | 12.624 | 7.00636 | 3.70% |
| 5 | 13.049 | 6.77933 | 9.60% |
| 6 | 13.644 | 6.48472 | 4.30% |
| 7 | 14.380 | 6.15464 | 3.10% |
| 8 | 14.804 | 5.97913 | 2.20% |
| 9 | 15.308 | 5.78332 | 12.30% |
| 10 | 16.458 | 5.38181 | 12.90% |
| 11 | 17.330 | 5.11302 | 5.30% |
| 12 | 17.754 | 4.99186 | 4.90% |
| 13 | 19.126 | 4.63669 | 4.60% |
| 14 | 19.984 | 4.43940 | 7.70% |
| 15 | 20.443 | 4.34085 | 7.30% |
| 16 | 20.751 | 4.27719 | 2.80% |
| 17 | 21.111 | 4.20488 | 7.00% |
| 18 | 21.451 | 4.13904 | 17.90% |
| 19 | 21.952 | 4.04574 | 2.80% |
| 20 | 22.284 | 3.98629 | 5.90% |
| 21 | 22.859 | 3.88719 | 2.00% |
| 22 | 23.652 | 3.75860 | 3.80% |
| 23 | 24.076 | 3.69338 | 1.70% |
| 24 | 25.611 | 3.47542 | 2.20% |
| 25 | 26.257 | 3.39137 | 4.40% |
| 26 | 26.824 | 3.32095 | 1.80% |
| 27 | 28.067 | 3.17665 | 2.40% |
| 28 | 31.375 | 2.84884 | 1.40% |

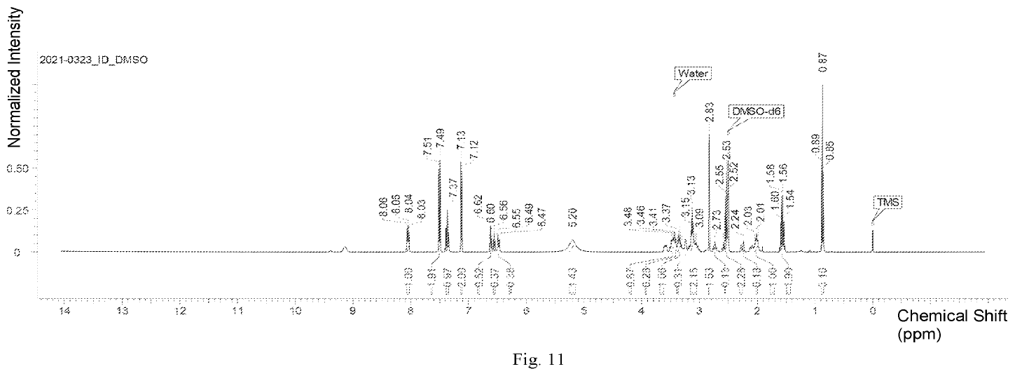

Proton NMR is conducted and it shows an estimated molar ratio of 1:2 lumateperone to acid, thus confirming that this is a 1:2 salt. The NMR spectrum is shown in FIG. 11.

### Example 5: 4-Ethylbenzenesulfonic Acid Salt

Initial screening results demonstrated the formation of a crystalline lumateperone 4-ethylbenzenesulfonate salt using a 1:2 molar ratio in both ethyl acetate solvent and toluene solvent, but initial XRPD showed that these solids had two different XPRD patterns. In addition, amorphous solids were obtained from the screen using 1:2 molar ratio in methanol and 1:2 molar ratio in methanol, these two amorphous XRPD patterns also being distinct. No solids were obtained using acetonitrile/water as solvent, or using toluene or ethyl acetate at a 1:1 molar ratio. Initial DSC suggested that this both crystalline solids were non-solvated, anhydrous crystals. DSC showed a single melting event at 153.2° C. for the ethyl acetate condition, and a single melting event at 157.6° C. for the toluene condition. Weight loss on TGA was zero for the ethyl acetate condition and 2.2% for the toluene condition.

The alternative reaction conditions described supra (50 mg scale, 50° C. for 2 hours) also resulted in a third crystalline 4-ethylbenzenesulfonate candidate salt. This salt was obtained using both ethyl acetate solvent and toluene solvent. The solid from the toluene condition was tested in DSC and TGA. DSC shows a single melting event at 141.5° C., and 0.6% mass loss on TGA (residual solvent).

The 100 mg scale-up was performed using each of the three initial conditions which produced crystalline solid, and a solid was obtained in each case having the same XRPD pattern as in the 25 mg initial screen. Polymorph 1 is obtained using a 1:2 molar ratio in ethyl acetate solvent; Polymorph 2 is obtained using a 1:2 molar ratio in toluene solvent; and Polymorph 3 is obtained using a 1:1 molar ratio in toluene solvent with heptane anti-solvent. DSC showed a single melting event for each solid: Polymorph 1, 147.0° C. (3.7% TGA weight loss); Polymorph 2, 156.5° C. (no TGA weight loss); Polymorph 3, 138.3° C. (1.4% TGA weight loss). The results are indicative of a non-solvated, anhydrous crystals with some residual solvent losses.

The XRPD patterns are shown in FIGS. 4A, 4B, and 4C, and the peak listings are shown in the tables below:

XRPD (Cu anode, Ni filter) for 4-ethylbenzenesulfonate
Salt Crystal Polymorph 1

| # | Angle | d Value | Rel. Intensity |
|---|---|---|---|
| 1 | 3.511 | 25.14320 | 100.00% |
| 2 | 3.684 | 23.96635 | 55.30% |
| 3 | 4.982 | 17.72257 | 14.80% |
| 4 | 6.917 | 12.76937 | 5.60% |
| 5 | 7.321 | 12.06545 | 5.30% |
| 6 | 10.354 | 8.53641 | 2.60% |
| 7 | 10.383 | 8.51268 | 2.80% |
| 8 | 12.488 | 7.08228 | 2.60% |
| 9 | 13.689 | 6.46357 | 4.10% |
| 10 | 14.269 | 6.20219 | 9.00% |
| 11 | 15.587 | 5.68057 | 40.60% |
| 12 | 17.499 | 5.06386 | 9.70% |
| 13 | 17.682 | 5.01186 | 8.80% |
| 14 | 18.581 | 4.77134 | 13.70% |
| 15 | 18.852 | 4.70347 | 13.50% |
| 16 | 20.127 | 4.40823 | 27.20% |
| 17 | 20.609 | 4.30628 | 13.50% |
| 18 | 20.833 | 4.26051 | 12.20% |
| 19 | 21.246 | 4.17848 | 7.60% |
| 20 | 22.016 | 4.03418 | 5.30% |
| 21 | 27.486 | 3.24248 | 4.50% |

XRPD (Cu anode, Ni filter) for 4-ethylbenzenesulfonate
Salt Crystal Polymorph 2

| # | Angle | d Value | Rel. Intensity |
|---|---|---|---|
| 1 | 3.694 | 23.89639 | 100.00% |
| 2 | 5.034 | 17.54009 | 24.00% |
| 3 | 7.319 | 12.06926 | 13.00% |
| 4 | 10.267 | 8.60911 | 6.10% |
| 5 | 11.190 | 7.90062 | 0.30% |
| 6 | 11.695 | 7.56092 | 7.00% |
| 7 | 13.798 | 6.41287 | 6.70% |
| 8 | 14.271 | 6.20112 | 16.40% |
| 9 | 14.794 | 5.98337 | 24.30% |
| 10 | 15.630 | 5.66494 | 74.70% |
| 11 | 16.492 | 5.37089 | 11.00% |
| 12 | 17.661 | 5.01793 | 27.40% |
| 13 | 18.800 | 4.71645 | 50.80% |
| 14 | 20.221 | 4.38793 | 53.10% |
| 15 | 20.575 | 4.31324 | 25.80% |
| 16 | 20.782 | 4.27078 | 34.30% |
| 17 | 21.075 | 4.21203 | 14.90% |
| 18 | 21.413 | 4.14627 | 16.10% |
| 19 | 22.019 | 4.03362 | 14.10% |
| 20 | 22.956 | 3.87095 | 14.00% |
| 21 | 24.085 | 3.69203 | 4.60% |
| 22 | 24.743 | 3.59528 | 13.20% |
| 23 | 25.281 | 3.52006 | 12.00% |
| 24 | 26.244 | 3.39300 | 0.90% |
| 25 | 27.703 | 3.21750 | 11.40% |

XRPD (Cu anode, Ni filter) for 4-ethylbenzenesulfonate
Salt Crystal Polymorph 3

| # | Angle | d Value | Rel. Intensity |
|---|---|---|---|
| 1 | 2.789 | 31.65519 | 30.70% |
| 2 | 5.569 | 15.85574 | 100.00% |
| 3 | 11.382 | 7.76773 | 16.90% |
| 4 | 12.090 | 7.31483 | 19.80% |
| 5 | 13.121 | 6.74217 | 21.00% |
| 6 | 15.965 | 5.54681 | 28.90% |
| 7 | 16.585 | 5.34081 | 57.90% |
| 8 | 17.487 | 5.06740 | 24.40% |
| 9 | 17.996 | 4.92527 | 27.00% |
| 10 | 18.865 | 4.70026 | 26.20% |
| 11 | 19.406 | 4.57029 | 21.80% |
| 12 | 20.542 | 4.32010 | 11.20% |
| 13 | 21.306 | 4.16702 | 20.10% |
| 14 | 22.536 | 3.94215 | 63.20% |
| 15 | 22.796 | 3.89784 | 40.40% |
| 16 | 23.272 | 3.81916 | 36.00% |
| 17 | 24.020 | 3.70193 | 50.10% |
| 18 | 25.453 | 3.49666 | 15.10% |
| 19 | 31.614 | 2.82786 | 5.90% |
| 20 | 34.006 | 2.63421 | 3.50% |

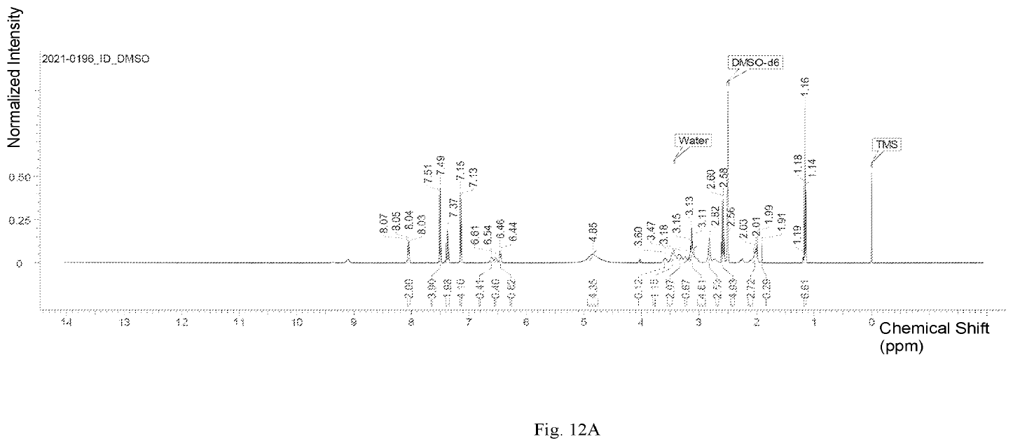

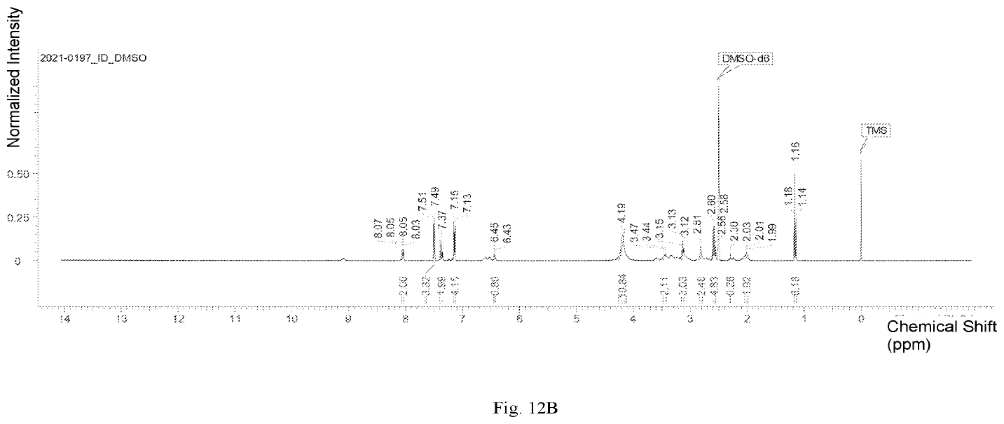

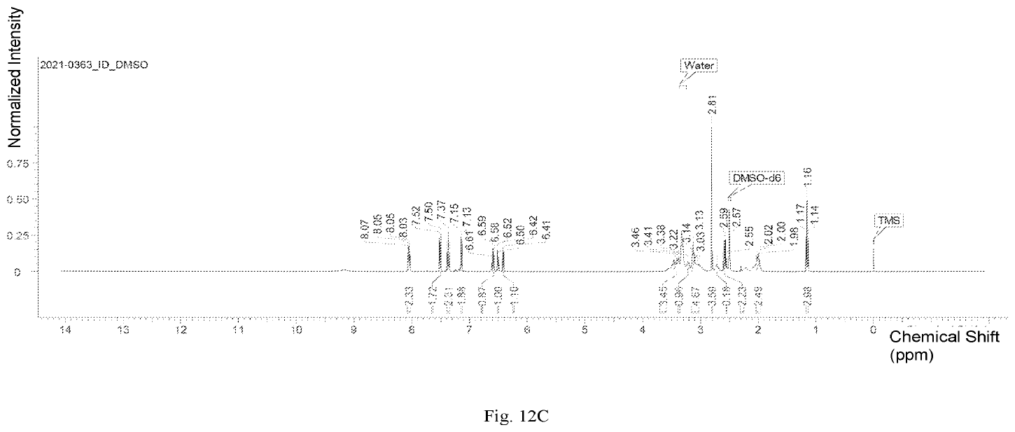

Proton NMR is conducted and it shows an estimated molar ratio of 1:2 lumateperone to acid for Polymorphs 1 and 2, and a 1:1 ratio for Polymorph 3. The NMR spectra are shown in FIGS. 12A, 12B and 12C.

### Example 6: 2-Naphthalenesulfonic Acid Salt

Initial screening results demonstrated the formation of a crystalline lumateperone 2-naphthalenesulfonate salt using a 1:2 molar ratio in ethyl acetate solvent. No significant solids were obtained using acetonitrile/water, methanol, or toluene as solvent at 1:1 or 1:2 molar ratio, nor at 1:1 molar ratio in ethyl acetate. Initial DSC suggested that this was a solvated crystal. DSC showed a desolvation event at 91.4° C., and no melting event, and TGA sowed a 6.0% weight loss, consistent with desolvation of the crystal.

The 100 mg scale-up was performed using ethyl acetate at a 1:2 molar ratio, and a solid was obtained having the same XRPD pattern as in the 25 mg initial screen. DSC on the solid showed a desolvation event at 108.5° C. and a single melting event at 162.1° C. TGA mass loss was 7.4%, consistent with desolvation of the crystal.

The XRPD pattern is shown in FIG. 5, and the peak listing is shown in the table below:

XRPD (Cu anode, Ni filter) for 2-
Naphthalenesulfonate Salt Crystal

| # | Angle | d Value | Rel. Intensity |
|---|---|---|---|
| 1 | 2.401 | 36.77034 | 32.50% |
| 2 | 7.045 | 12.53758 | 16.40% |
| 3 | 14.063 | 6.29268 | 10.20% |
| 4 | 14.906 | 5.93853 | 27.30% |
| 5 | 15.477 | 5.72064 | 26.40% |
| 6 | 17.204 | 5.15024 | 25.70% |
| 7 | 17.493 | 5.06578 | 21.00% |
| 8 | 19.018 | 4.66279 | 26.30% |
| 9 | 20.323 | 4.36625 | 40.90% |
| 10 | 20.418 | 4.34608 | 39.50% |
| 11 | 20.780 | 4.27126 | 84.70% |
| 12 | 20.842 | 4.25855 | 100.00% |
| 13 | 21.247 | 4.17835 | 28.40% |
| 14 | 23.846 | 3.72847 | 21.10% |
| 15 | 26.939 | 3.30704 | 9.30% |
| 16 | 27.208 | 3.27490 | 28.90% |

Proton NMR is conducted and it shows an estimated molar ratio of 1:1 lumateperone to acid, thus confirming that this is a 1:1 salt.

### Example 7: Benzenesulfonic Acid Salt

Initial screening results demonstrated the formation of a crystalline candidate salt using a 1:2 molar ratio in both ethyl acetate solvent and toluene solvent, but initial XRPD showed that these solids had two different XPRD patterns. In addition, an amorphous solid was obtained from the screen using 1:1 molar ratio in ethyl acetate. No solids were obtained using acetonitrile/water or methanol as solvent, or using toluene at a 1:1 molar ratio. Initial DSC suggested that both of these crystalline solids were solvated crystals. DSC showed a desolvation events at 73.9° C. and 110.6° C., as well as a single melting event at 172.6° C. for the ethyl acetate condition, and a desolvation event at 75.1° C. and a single melting event at 174.1° C. for the toluene condition. Weight loss on TGA was 4.9% for the ethyl acetate condition and 3.6% for the toluene condition, both consistent with solvated crystals.

The 100 mg scale-up was performed using each of the three initial conditions which produced solid, but none of the original products were reproduced. Instead, each of the three conditions-including the one which provided an amorphous solid at 25 mg scale-resulted in a distinct crystalline salt XRPD pattern at the 100 mg scale. Polymorph 1 is obtained using a 1:2 molar ratio in ethyl acetate solvent; Polymorph 2 is obtained using a 1:2 molar ratio in toluene solvent; and Polymorph 3 is obtained using a 1:1 molar ratio in ethyl acetate solvent. Polymorph 1 shows a desolvation event at 96.0° C. and a melting event at 109.5° C., with 6.3% TGA weight loss. This is consistent with a solvated crystal. Polymorph 2 shows a single melting event at 131.3° C. and no TGA weight loss, consistent with a non-solvated, anhydrous crystal. Polymorph 3 shows two melting events, at 109.6 and 125.9° C., with no TGA weight loss. This is also indicative of a non-solvated, anhydrous crystal.

The XRPD patterns are shown in FIGS. 6A, 6B, and 6C, and the peak listings are shown in the tables below:

XRPD (Cu anode, Ni filter) for benzenesulfonate
Salt Crystal Polymorph 1

| # | Angle | d Value | Rel. Intensity |
|---|---|---|---|
| 1 | 5.807 | 15.20724 | E+03 70.20% |
| 2 | 5.879 | 15.02115 | 54.50% |
| 3 | 8.810 | 10.02881 | 11.60% |
| 4 | 11.418 | 7.74363 | 15.30% |
| 5 | 11.733 | 7.53663 | 17.30% |
| 6 | 12.021 | 7.35627 | 45.10% |
| 7 | 12.389 | 7.13857 | 38.00% |
| 8 | 13.260 | 6.67196 | 20.40% |
| 9 | 15.777 | 5.61243 | 36.30% |
| 10 | 15.944 | 5.55401 | 80.80% |
| 11 | 16.114 | 5.49583 | 83.80% |
| 12 | 16.652 | 5.31950 | 60.00% |
| 13 | 17.003 | 5.21066 | 83.50% |
| 14 | 17.587 | 5.03871 | 13.90% |
| 15 | 17.812 | 4.97576 | 20.60% |
| 16 | 18.016 | 4.91984 | 8.20% |
| 17 | 18.283 | 4.84860 | 45.00% |
| 18 | 18.658 | 4.75200 | 15.90% |
| 19 | 19.358 | 4.58153 | 58.70% |
| 20 | 19.927 | 4.45216 | 38.30% |
| 21 | 20.196 | 4.39339 | 8.50% |
| 22 | 20.529 | 4.32286 | 19.20% |
| 23 | 21.207 | 4.18609 | 13.40% |
| 24 | 22.598 | 3.93146 | 100.00% |
| 25 | 22.980 | 3.86708 | 59.20% |
| 26 | 23.301 | 3.81451 | 51.30% |
| 27 | 23.718 | 3.74831 | 22.40% |
| 28 | 24.086 | 3.69188 | 61.20% |
| 29 | 24.435 | 3.63990 | 9.20% |
| 30 | 25.522 | 3.48730 | 16.00% |
| 31 | 26.195 | 3.39921 | 15.90% |
| 32 | 26.325 | 3.38274 | 15.90% |
| 33 | 27.246 | 3.27051 | 10.30% |
| 34 | 27.962 | 3.18835 | 12.70% |
| 35 | 30.132 | 2.96347 | 7.50% |

XRPD (Cu anode, Ni filter) for benzenesulfonate
Salt Crystal Polymorph 2

| # | Angle | d Value | Rel. Intensity |
|---|---|---|---|
| 1 | 4.655 | 18.96770 | 37.60% |
| 2 | 5.901 | 14.96484 | 58.00% |
| 3 | 11.434 | 7.73279 | 20.50% |
| 4 | 12.022 | 7.35562 | 52.90% |
| 5 | 12.404 | 7.13035 | 44.40% |
| 6 | 13.269 | 6.66704 | 17.50% |
| 7 | 13.893 | 6.36900 | 39.00% |
| 8 | 14.765 | 5.99485 | 17.40% |
| 9 | 15.290 | 5.79017 | 34.50% |
| 10 | 15.964 | 5.54740 | 87.00% |
| 11 | 16.112 | 5.49647 | 87.10% |
| 12 | 16.673 | 5.31286 | 76.80% |
| 13 | 17.025 | 5.20374 | 74.90% |
| 14 | 17.359 | 5.10456 | 63.20% |
| 15 | 17.848 | 4.96561 | 29.50% |
| 16 | 17.949 | 4.93789 | 31.50% |
| 17 | 18.290 | 4.84677 | 53.30% |
| 18 | 18.649 | 4.75407 | 26.30% |
| 19 | 19.374 | 4.57783 | 54.50% |
| 20 | 19.934 | 4.45050 | 32.50% |
| 21 | 20.518 | 4.32524 | 58.60% |
| 22 | 21.215 | 4.18450 | 11.00% |
| 23 | 21.845 | 4.06526 | 15.40% |
| 24 | 22.603 | 3.93072 | 100.00% |
| 25 | 22.980 | 3.86696 | 66.10% |
| 26 | 23.317 | 3.81191 | 45.50% |
| 27 | 23.753 | 3.74293 | 27.70% |
| 28 | 24.099 | 3.68997 | 69.70% |
| 29 | 25.119 | 3.54231 | 19.50% |
| 30 | 26.174 | 3.40189 | 26.60% |
| 31 | 27.970 | 3.18742 | 12.60% |
| 32 | 30.127 | 2.96396 | 6.90% |

XRPD (Cu anode, Ni filter) for benzenesulfonate
Salt Crystal Polymorph 3

| # | Angle | d Value | Rel. Intensity |
|---|---|---|---|
| 1 | 5.240 | 16.85241 | 89.90% |
| 2 | 5.756 | 15.34242 | 100.00% |
| 3 | 14.387 | 6.15169 | 73.40% |
| 4 | 16.165 | 5.47865 | 47.20% |
| 5 | 16.713 | 5.30036 | 45.40% |
| 6 | 18.653 | 4.75319 | 21.10% |
| 7 | 19.249 | 4.60734 | 31.00% |
| 8 | 19.441 | 4.56222 | 62.80% |
| 9 | 20.697 | 4.28806 | 50.40% |
| 10 | 20.839 | 4.25914 | 50.10% |
| 11 | 21.084 | 4.21030 | 28.80% |
| 12 | 22.806 | 3.89608 | 48.00% |
| 13 | 22.907 | 3.87919 | 46.20% |
| 14 | 23.212 | 3.82890 | 71.20% |
| 15 | 23.874 | 3.72418 | 30.60% |
| 16 | 24.695 | 3.60221 | 99.90% |
| 17 | 25.066 | 3.54981 | 30.10% |
| 18 | 25.127 | 3.54131 | 24.10% |
| 19 | 26.712 | 3.33459 | 6.90% |
| 20 | 28.440 | 3.13583 | 14.30% |
| 21 | 33.755 | 2.65326 | 6.00% |
| 22 | 37.907 | 2.37161 | 11.30% |

Proton NMR is conducted and it shows an estimated molar ratio of 1:1 lumateperone to acid for each of Polymorphs 1, 2, and 3. The NMR spectra are shown in FIGS. 12A, 12B and 12C.

A lumateperone besylate salt was previously reported in WO 2020/112941 (Teva Pharmaceuticals, Inc; Teva Czech Industries SRO). However, the besylate salt reported in this application has a melting event at a much higher temperature, 173-174° C., compared to Polymorphs 1, 2 and 3, above. The XRPD patterns for the Teva crystal are also dissimilar to Polymorphs 1, 2 and 3, above.

### Example 8: Pentane-1-Sulfonic Acid Salt

Initial screening results demonstrated the formation of a crystalline lumateperone pentane-1-sulfonic salt using a 1:2 molar ratio in ethyl acetate solvent. No solids were obtained using acetonitrile/water, methanol, or toluene as solvent with 1:1 or 1:2 molar ratio, nor in ethyl acetate at a 1:1 molar ratio. Initial DSC suggested that this was a non-solvated, anhydrous crystal. DSC showed no events, and TGA showed 1.4% weight loss, consistent with residual solvent.

The 100 mg scale-up was performed using ethyl acetate at a 1:2 molar ratio, and a solid was obtained having the same XRPD pattern as in the 25 mg initial screen. DSC on the solid showed a single melting event at 141.3° C. TGA mass loss was 0.9%, consistent with residual solvent. This is indicative of a non-solvated, anhydrous crystal.

The XRPD pattern is shown in FIG. 7, and the peak listing is shown in the table below:

XRPD (Cu anode, Ni filter) for Pentane-1-sulfonate Salt Crystal

| # | Angle | d Value | Rel. Intensity |
|---|---|---|---|
| 1 | 2.086 | 42.32365 | 2.40% |
| 2 | 3.777 | 23.37371 | 100.00% |
| 3 | 7.487 | 11.79790 | 19.80% |
| 4 | 11.225 | 7.87635 | 5.70% |
| 5 | 14.766 | 5.99468 | 32.90% |
| 6 | 16.224 | 5.45895 | 20.60% |
| 7 | 16.561 | 5.34869 | 28.00% |
| 8 | 17.339 | 5.11045 | 17.80% |
| 9 | 17.757 | 4.99095 | 29.40% |
| 10 | 18.639 | 4.75678 | 14.70% |
| 11 | 19.663 | 4.51123 | 17.30% |
| 12 | 20.014 | 4.43290 | 54.80% |
| 13 | 20.344 | 4.36175 | 28.70% |
| 14 | 20.782 | 4.27088 | 16.80% |
| 15 | 21.353 | 4.15784 | 16.10% |
| 16 | 21.738 | 4.08517 | 20.60% |
| 17 | 22.567 | 3.93679 | 15.00% |
| 18 | 25.287 | 3.51922 | 17.40% |
| 19 | 38.261 | 2.35050 | 3.90% |

Proton NMR is conducted and it shows an estimated molar ratio of 1:1.8 lumateperone to acid, thus confirming that this is a 1:2 salt.

### Example 9: Heptane-1-Sulfonic Acid Salt

Initial screening results demonstrated the formation of a crystalline lumateperone heptane-1-sulfonic salt using a 1:2 molar ratio in ethyl acetate solvent. No solids were obtained using acetonitrile/water, methanol, or toluene as solvent with 1:1 or 1:2 molar ratio, nor in ethyl acetate at a 1:1 molar ratio. Initial DSC suggested that this was a non-solvated, anhydrous crystal. DSC showed a melting event at 151.8° C., and TGA showed no weight loss. Addition of anti-solvent to the 1:2 molar ratio toluene vial, however, resulted in formation of a solid with an XRPD pattern distinct from the ethyl acetate condition. This DSC of this solid showed a desolvation event at 105° C., and a TGA mass loss of 1.1%.

The 100 mg scale-up was performed using ethyl acetate at a 1:2 molar ratio, and a solid was obtained having the same XRPD pattern as in the 25 mg initial screen under this condition. DSC on the solid showed a single melting event at 150.8° C. TGA showed no mass loss. This is indicative of a non-solvated, anhydrous crystal.

The XRPD pattern is shown in FIG. 8, and the peak listing is shown in the table below:

XRPD (Cu anode, Ni filter) for Heptane-1-sulfonate Salt Crystal

| # | Angle | d Value | Rel. Intensity |
|---|---|---|---|
| 1 | 3.491 | 25.29231 | 100.00% |
| 2 | 6.836 | 12.92058 | 5.90% |
| 3 | 7.435 | 11.88044 | 5.10% |
| 4 | 10.282 | 8.59661 | 2.50% |
| 5 | 13.686 | 6.46513 | 3.40% |
| 6 | 14.175 | 6.24316 | 11.20% |
| 7 | 14.883 | 5.94771 | 3.60% |
| 8 | 15.750 | 5.62216 | 7.30% |
| 9 | 16.320 | 5.42709 | 6.00% |
| 10 | 16.737 | 5.29276 | 7.80% |
| 11 | 17.121 | 5.17487 | 9.00% |
| 12 | 17.617 | 5.03024 | 14.20% |
| 13 | 18.042 | 4.91282 | 12.60% |
| 14 | 18.345 | 4.83224 | 10.20% |
| 15 | 19.220 | 4.61410 | 5.80% |
| 16 | 19.904 | 4.45723 | 26.70% |
| 17 | 20.184 | 4.39595 | 32.60% |
| 18 | 20.498 | 4.32935 | 7.80% |
| 19 | 21.602 | 4.11051 | 2.30% |
| 20 | 22.401 | 3.96562 | 9.70% |
| 21 | 23.223 | 3.82713 | 6.80% |
| 22 | 24.178 | 3.67808 | 4.80% |
| 23 | 24.844 | 3.58092 | 16.70% |
| 24 | 26.327 | 3.38255 | 2.30% |
| 25 | 27.563 | 3.23360 | 2.70% |
| 26 | 30.590 | 2.92015 | 2.50% |
| 27 | 31.762 | 2.81500 | 1.60% |
| 28 | 32.886 | 2.72129 | 2.20% |
| 29 | 33.600 | 2.66507 | 2.20% |

Proton NMR is conducted and it shows an estimated molar ratio of 1:1.8 lumateperone to acid, thus confirming that this is a 1:2 salt.

### Example 10: Aqueous Solubility Determination

The aqueous solubility of the successfully reproduced salts is determined by shaking a saturated solution in water. The solubility at pH 7.4 is determined by shaking a saturated solution in a phosphate buffered system. The samples are prepared by the addition of a known amount of water (100-200 μl) and subsequent slurrying with the solid for 24 hours. All samples are then filtered using syringe filters to remove undissolved solids, and then the filtrates are diluted with methanol/acetonitrile (1:1 v/v). LC analysis of the samples is performed, and a calibration line is used for the determination of the solubilities. The results are shown in the table below in mg/mL:

| Salt | Aqueous Solubility | pH 7.4 Solubility |
|---|---|---|
| Example 2 | 0.006 | 0.24 |
| Example 3 | 0.77 | 0.07 |
| Example 4 | 1.62 | 0.17 |
| Example 5, Polymorph 1 | 4.75 | 2.03 |
| Example 5, Polymorph 2 | 3.73 | 1.64 |
| Example 5, Polymorph 3 | 3.35 | 0.52 |
| Example 6 | 11.84 | 5.18 |
| Example 7, Polymorph 1 | 29.66 | 13.90 |
| Example 7, Polymorph 2 | 14.15 | 11.06 |
| Example 7, Polymorph 3 | 15.84 | 2.14 |
| Example 8 | 18.77 | 7.76 |
| Example 9 | 4.30 | 1.14 |
| Lumateperone monotosylate | 1.97 | 0.72 |

It is shown that several of the salts prepared according to the present disclosure have surprisingly low aqueous solubility. These low-solubility salts would therefore be particularly suited to formulation as an aqueous long-acting injectable composition. Injection of such a composition, subcutaneously or intramuscularly, would provide a largely insoluble depot of the active agent, lumateperone, which would slowly dissolve to release lumateperone free base according to the solubility kinetics of the salt. A suitable formulation for subcutaneous or intramuscular injection is provided in the following table.

| Component | Weight % |
|---|---|
| Lumateperone Salt (e.g., salt of | 1-50% |
| Examples 2-9) | |
| Thickening agent (e.g., sodium | 0-20% |
| carboxymethyl cellulose | |
| Bulking agent (e.g., mannitol) | 0-20% |
| Buffer (e.g., sodium phosphates) | 0.01-5% |
| pH-adjusting agent (e.g., NaOH) | <1% |
| Water | Q.S. (e.g., 20-90% |

One or more formulations according to the above Table are prepared by combining the lumateperone salt, the thickening agent(s) (e.g., sodium carboxymethyl cellulose), and the bulking agents (e.g., mannitol), in water (e.g., sterile water for injection). The solid materials may be ground or milled to an appropriate particle size individually or together. The solids are suspended in water, and the pH is adjusted as desired, for example, to between pH 6.5 and 7.5, e.g., 7 to 7.5 or 7.3-7.4. Further studies are to be conducted to confirm the plasma and CSF pharmacokinetics of such formulations.

1. A solid, crystalline salt of lumateperone having an aqueous solubility of less than 2 mg/mL at pH 7, or less than 1 mg/mL at pH 7.4.

2. Lumateperone in the form of a salt selected from a 4-octylbenzenesulfonic acid addition salt, a 4-tert-butylbenzenesulfonic acid addition salt, a 4-propylbenzenesulfonic acid addition salt, a 4-ethylbenzenesulfonic acid addition salt, a 2-naphthalenesulfonic acid addition salt wherein the salt is a solvate, a solid crystalline benzenesulfonic acid addition salt characterized by a DSC thermogram lacking an endothermic event at 172-176° C., and an alkylsulfonic acid addition salt (e.g., a pentane-1-sulfonate, or a heptane-1-sulfonate).

3. Lumateperone in the form of an acid addition salt with a benzenesulfonic acid substituted by one, two, or three groups R, wherein each R is independently a C₁₋₁₂alkyl group, provided that the acid is not p-toluenesulfonic acid, 4-ethylbenzenesulfonic acid, 4-propylbenzenesulfonic acid, 4-t-butylbenzenesulfonic acid, or 4-octylbenzenesulfonic acid

4. The salt according to claim 2 in crystalline form.

5. The salt according to claim 2, wherein the salt is a 4-octylbenzenesulfonate salt having a 1:1 molar ratio or a 1:2 molar ratio of lumateperone free base to 4-octylbenzenesulfonic acid.

6. A process for the production of a salt according to claim 2, comprising the steps of:
(a) reacting lumateperone free base with corresponding acid, together with an organic solvent (e.g., comprising ethanol, methanol, toluene, ethyl acetate, cyclopentylmethyl ether (CPME), methyl tert-butyl ether (MTBE), methyl ethyl ketone (MEK), acetonitrile, 1-butanol, water, or mixtures thereof), for example, wherein the acid and lumateperone are in a molar ratio of from 1:1 to 1:2, or about 1:1, or about 1:2;
optionally at a temperature between 0° C. and 100° C.; and
(b) optionally, subject the resulting mixture to a thermocycling protocol (e.g., elevating the temperature to above 50° C., then cooling to 0° C., and optionally repeating this heating and cooling cyclically), or cooling the mixture from its reaction temperature to 5° C. or less; and
(c) optionally, diluting the resulting mixture with an anti-solvent, e.g., when the organic solvent is methanol, ethanol, 1-butanol, acetonitrile, or a solvent/water mixture, and the anti-solvent is water, or wherein the organic solvent is toluene, ethyl acetate, CPME, MTBE, MEK, or 1-butanol, and the anti-solvent is heptane or hexane;
optionally at a temperature between 0° C. and 100° C.; and
(d) optionally performing a second thermocycling protocol (e.g., elevating the temperature to above 50° C., then cooling to 0° C., and optionally repeating this heating and cooling cyclically), or cooling the mixture from its reaction temperature to 5° C. or less; and
(e) recovering the salt thus formed, e.g., recovering a salt according to claim 2.

7. A method of purifying lumateperone, in free or salt form, comprising reacting a crude solution of lumateperone free base with an acid to form a salt according to claim 2, and recovering the salt thus formed, and optionally converting the salt thus formed back to lumateperone free base or to any other salt form of lumateperone (e.g., a lumateperone monotosylate salt).

8. A pharmaceutical composition comprising a salt according to claim 2 as active ingredient, in combination or association with a pharmaceutically acceptable diluent or carrier.

9. (canceled)

10. A long-acting injectable pharmaceutical composition comprising an acid addition salt of lumateperone according to claim 2.

11. The composition according to claim 9, wherein the composition comprises water (e.g., sterile water for injection) and one or more excipients (e.g., water-soluble excipients), such as, thickening agents, buffering agents, osmotic agents, surfactants, and antioxidants.

12. The composition according to claim 11, wherein the one or more water-soluble excipients comprise thickening agents selected from carboxymethyl cellulose, sodium carboxymethyl cellulose, hydroxypropyl cellulose (HPC), hydroxyethyl cellulose (HEC), hydroxypropyl methyl cellulose (HPMC), hydroxypropyl ethyl cellulose (HPEC), microcrystalline cellulose, non-crystalline cellulose, polyacrylate polymers, polyvinylpyrrolidones, polyvinyl alcohols, and polyethylene glycols, and/or surfactants selected from sorbitan esters (e.g., sorbitan laurate, sorbitan oleate, sorbitan palmitate, sorbitan stearate), polyoxyethylene sorbitan fatty acid esters (e.g., polysorbate 20, polysorbate 40, polysorbate 60, polysorbate 80), polyoxyethylene alkyl ethers, fatty acid esters (e.g., glycerol monostearate, glycerol monolaurate), poloxamers, and fatty alcohols (e.g., stearyl alcohol, cetyl alcohol, cetostearyl alcohol), and bulking agents selected from mannitol, sucrose, fructose, maltose, xylitol, glucose, starches, sorbitol, magnesium aluminum silicate, and silica (e.g., colloidal silica).

13. The composition according to claim 11, wherein the composition comprises the acid addition salt of lumateperone (e.g., lumateperone 4-octylbenzenesulfonate or lumateperone 4-tert-butylbenzenesulfonate), water (e.g., sterile water for injection), a thickening agent (e.g., sodium carboxymethylcellulose), a bulking agent (e.g., mannitol), a non-ionic surfactant (e.g., polysorbate 80), and optionally one or more pH-adjusting or buffering agents (e.g., NaOH or HCl, and/or sodium or potassium phosphates).

14. The composition according to claim 11, wherein the composition comprises the acid addition salt of lumateperone (e.g., lumateperone 4-octylbenzenesulfonate or lumateperone 4-tert-butylbenzenesulfonate), water (e.g., sterile water for injection), a thickening agent (e.g., sodium carboxymethylcellulose), a bulking agent (e.g., mannitol), and optionally one or more pH-adjusting or buffering agents (e.g., sodium or potassium phosphates, NaOH and/or HCl, and/or sodium or potassium phosphates).

15. The composition according to claim 9, wherein the composition has the following components:

| | |
|---|---|
| Component | Weight % |
| Lumateperone Salt (e.g., salt of | 1-50% | (e.g., 10-40%) |
|---|---|---|
| Examples 2-9) | | |
| Thickening agent (e.g., sodium | 0-20% | (e.g., 1-20%) |
| carboxymethyl cellulose | | |
| Bulking agent (e.g., mannitol) | 0-20% | (e.g., 1-20%) |
| Buffer (e.g., sodium phosphates) | 0.01-5% | (e.g., 0.1-1%) |
| pH-adjusting agent (e.g., NaOH) | <1% | |
| Water | Q.S. | (e.g., 20-90% |

16. The composition according to claim 9, wherein the composition is a suspension.

17. The composition according to claim 9, wherein the composition is manufactured as a dried solid comprising the acid addition salt of lumateperone and one or more diluents, carriers, or excipients (e.g., water-soluble excipients), and wherein prior to administration, the solid is reconstituted with sterile water for injection to form the long-acting injectable pharmaceutical composition.

18. A method for the prophylaxis or treatment of a human suffering from a disease or abnormal condition involving or mediated by the 5-HT_{2A} receptor, serotonin transporter (SERT), and/or dopamine D₁/D₂ receptor signaling pathways comprising administering to said human an effective amount of a salt according to claim 2.
